(19) United States
(12) Patent Application Publication
(10) Pub. No.: US 2024/0116923 A1
(43) Pub. Date: Apr. 11, 2024

(54) USE OF INDOLE, 6- AND 7-AZAINDOLE DERIVATIVES AS INHIBITORS OF FERROPTOSIS REGULATED CELL DEATH

(71) Applicants: Seabelife, Roscoff (FR); Centre National de la Recherche Scientifique (CNRS), Paris (FR); Sorbonne Universite, Paris (FR); INSTITUT NATIONAL DE LA SANTÉ ET DE LA RECHERCHE MÉDICALE (INSERM), PARIS (FR); Université Claude Bernard Lyon 1, Villeurbanne (FR); Universite de Rennes 1, Rennes (FR)

(72) Inventors: Stéphane BACH, Sibiril (FR); Arnaud COMTE, Lyon (FR); Claire DELEHOUZE, La Roche Maurice (FR); Marie-Thérèse DIMANCHE-BOITREL, Melesse (FR); Peter GOEKJIAN, Villeurbanne (FR)

(73) Assignees: Seabelife, Roscoff (FR); Centre National de la Recherche Scientifique (CNRS), Paris (FR); Sorbonne Universite, Paris (FR); Institut National de la Santé et de la Recherche Médicale (INSERM), Paris (FR); Université Claude Bernard Lyon 1, Villeurbanne (FR); Universite de Rennes 1, Rennes (FR)

(21) Appl. No.: 18/273,878

(22) PCT Filed: Jan. 25, 2022
(86) PCT No.: PCT/EP2022/051650

§ 371 Date: Jul. 24, 2023

(30) Foreign Application Priority Data
Jan. 25, 2021 (EP) 21305085.9

(51) Int. Cl. C07D 471/04; A61K 31/404; A61K 31/437; A61K 31/444; A61K 31/4545; A61K 31/496; A61K 31/5377; A61P 39/06; C07D 209/12

(52) U.S. Cl. CPC C07D 471/04; A61K 31/404; A61K 31/437; A61K 31/444; A61K 31/4545; A61K 31/496; A61K 31/5377; A61P 39/06; C07D 209/12

(57) ABSTRACT

The present invention relates to a compound of the following general formula (I) or a pharmaceutically acceptable salt and/or solvate thereof, for use as drug for inhibiting ferroptosis.

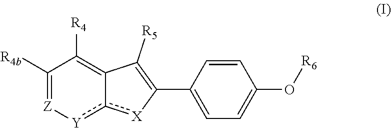

### CROSS-REFERENCE TO RELATED APPLICATIONS

This application is a U.S. National Stage Application pursuant to 35 U.S.C. § 371 of International Patent Application PCT/EP2022/051650, filed on Jan. 25, 2022, and published as WO 2022/157392 on Jul. 28, 2022, which claims priority to European Patent Application 21305085.9, filed on Jan. 25, 2021, all of which are incorporated herein by reference in their entireties for all purposes.

The present invention relates to a compound for use as a drug for inhibiting ferroptosis, more particularly, for preventing and/or treating disorders associated with ferroptosis.

Ferroptosis is a new type of non-apoptotic regulated cell death that was first described in 2012, and usually involves high intracellular levels of free iron and lipid peroxidation. This death pathway is directly linked to the ability of the cell to regulate its internal oxidative stress, notably via the activity of the lipid repair enzyme glutathione peroxidase 4 (GPX4). The failure of the glutathione-dependent antioxidant defenses causes an accumulation of lipid-based reactive oxygen species (ROS), which result notably from lipid peroxidation by Fe²⁺, through the Fenton's reaction, leading to membrane damage and cell death.

Recent studies show that ferroptosis is involved in the pathophysiology of many human diseases [Li et al., Cell Death Dis., 2020, 11(88); Tang et al., Cell Research, 2021, 31:107-125; Sun et al., Biomed. Pharmacother., 2020, 127, 110108], affecting the heart, the brain, the eyes, the liver, the skin, the kidneys, the lungs, the bowel or the whole body. Ferroptosis involves three primary metabolisms including thiol, lipid and iron leading to an iron-dependent generation of lipid peroxidation and, ultimately to cell death.

Hallmarks of ferroptosis were used as key elements to define ferroptosis-associated disease biomarkers. Ferroptosis is an iron-dependent regulated tissue necrosis mainly caused by unrestricted lipid peroxidation and subsequent membrane damage. The modifications of the physiological levels of the following components were reported as associated with ferroptosis: iron, reactive oxygen species, ROS (including lipid ROS such as 4-Hydroxynonenal (4-HNE) and malondialdehyde (MDA), and oxidized phospatidylethanolamine (oxPE) species followed by oxidized phosphatidylserine (oxPS) and oxidized phosphatidylinositol (oxPI) [Wiernicki et al., Cell Death Dis., 2020, 11(922)]) and related peroxide detoxification molecules (including the thiol-containing compound gluthation, GSH, or Coenzyme Q10, also known as ubiquinone), and the long-chain-fatty-acid-CoA ligase 4 (ACSL4) [Chen X. et al., Front. in Cell and Dev. Biol., 2021, 9(637162)]. These key biochemical ferroptosis biomarkers can be measured and quantified by assays in bodily fluids (blood, plasma, serum, urine, cerebrospinal fluid) or highlighted by immunohistochemistry labeling on biopsies of damaged tissues.

Depending on both the pathology and the damaged organ, several among ferroptosis-associated biomarkers could vary (increase > or decrease <) in quantity and/or activity relative to normal physiological thresholds. Here we only described reference values for serum:
(1) Iron metabolism (by measuring iron and ferritin levels in serum) is over the physiological thresholds (serum iron, in male >180 μg/dl, in female >160 μg/dl; [Pagana et al., Mosby's Diagnostic and Laboratory Test Reference—Elsevier eBook on VitalSource, 14th Edition, Elsevier, 2019, ISBN: 9780323609678]), ferritin, in male >300 ng/ml, in female >200 ng/ml, [Wang et al., Biochim Biophy. Acta, 2010, 1800(8): 760-769]);
(2) Glutathione redox status (by measuring reduced glutathione (GSH) and oxidized glutathione (GSSG) as well as glutathione peroxidase activity (GPx) in plasma using ELISA) (GSH<717 μmol/L, GSSG>5.32 μmol/L; ratio GSH/GSSG<156; GPx, in male <20 UI/gHb, in female <26 UI/gHb), [Haleng J. et al., Rev. Med. Liege, 2007]);
(3) Oxidative stress (by measuring levels of total Q10 and reduced and active form of Q10 (Q10H2) in plasma (in male Q10<3.44 μmol/l and Q10H2<3.04 μmol/l; in female Q10<1.88 μmol/l and Q10H2<1.64 μmol/l, [Kaikkonen et al., Scand J Clin Lab Invest, 1999, 59: 457-466]);
(4) Lipid peroxidation (measured by detection of 4-Hydroxynonenal (4-HNE) and malondialdehyde (MDA) adducts) is over the physiological thresholds (>10 μmol/L for 4-HNE [Chen and Niki, IUBMB Life, 2008, 58(372-373)] and >3 μmol/L for MDA using thiobarbituric acid method [Banjare et al., J Sci. Soc., 2017; 44(137-9)]).
   Note here that the upregulation of ACSL4 enzyme level in damaged organ tissues was also reported as putative biomarker of ferroptosis (ACSL4 expression can be monitored by transcriptomic and proteomic approaches).
   Pathologies associated with ferroptosis affecting the heart include myocardial ischemia-reperfusion injury, notably occurring after artery ligation, and cardiomyopathy, notably doxorubicin-induced cardiomyopathy [Li et al., 2020], among others [Li et al., Free Radic. Biol. Med., 2020, 160, 303-318; Qin et al., Biomed. Pharmacother., 2021, 141, 111872].

Pathologies associated with ferroptosis affecting the brain include strokes, notably ischemic stroke [Li et al., 2020] or hemorrhagic stroke [Li et al., JCI Insight, 2017, 2(7):e90777], traumatic brain injury [Xie et al., CNS Neurosci Ther., 2019, 25:465-475], contusion spinal cord injury [Zhang et al., Neural Regen. Res., 2019, 14(3):532] and neurodegenerative disorders, in particular chronic neurodegenerative disorders, more particularly Alzheimer's disease [Li et al., 2020], Huntington's disease [Mi et al., Neuromolecular Med., 2019, 21, 110-119], Parkinson's disease [Do Van et al., Neurobiol Dis., 2016, 94: 169-78], amyotrophic lateral sclerosis (Charcot's disease) [Li et al., 2020], Friedreich's ataxia [Cotticelli et al., J Pharmacol Exp Ther., 2019, 369(1): 47-54], periventriculor leukomalacia [Skouta et al., J. Am. Chem. Soc., 2014, 136, 4551-4556] and dementia, which may be linked to one or several of the previous pathologies.

Pathologies associated with ferroptosis affecting the eyes include retinal disorders, notably Stargardts' disease and age-related macular degeneration (AMD), in particular dry AMD [Sun et al., Invest Ophth Vis Sci., 2018, 59(9), 2482; Chen et al., J Biol. Chem., 2021, 296, 100187].

Pathologies associated with ferroptosis affecting the liver include chronic liver diseases and acute liver failure. Among chronic liver diseases, mention should be made of non-alcoholic steatohepatitis (NASH) [Qi et al., Am J Pathol., 2020, 190(1)], chronic infections such as hepatitis B and C [Cappelletti et al., Int J Mol Sci., 2020, 21(14)] and alcoholic liver disease [Zhou et al., Hepatol Commun., 2019, 3(5)]. Acute liver failure may notably result from a drug-induced liver injury (DILI), such as acetaminophem (APAP)-induced liver injury [Yamada et al., Cell Death Dis., 2020, 11(2)], or from an ischemia-reperfusion injury induced by a septic or hemorrhagic shock [Friedmann Angeli et al., Nat Cell Biol., 2014, 16(12):1180-91].

Pathologies associated with ferroptosis affecting the skin include skin inflammatory diseases, such as psoriasis [Li et al., Cell Death Dis., 2020, 11(88)], and toxic epidermal necrolysis (Lyell syndrome) [Zhang et al., J Invest Dermatol., 2020, 140(7), S79].

Pathologies associated with ferroptosis affecting the kidneys include acute kidney injury (AKI), such as crystal (oxalate)-, folic acid (FA)-induced AKI [Martin-Sanchez et al., 2017] and cisplatin-induced AKI [Deng et al., J Clin Invest., 2019, 129(11); Mishima et al., J Am Soc Nephrol., 2020, 31(2); Hu et al., Cell Death Dis., 2020, 11(1)], renal ischemia-reperfusion injury [Li et al., 2020], and acute tubular necrosis [Friedmann Angeli et al., 2014].

Pathologies associated with ferroptosis affecting the lungs include chronic obstructive pulmonary disease (COPD) [Yoshida et al., Nat Commun., 2019, 10, 3145], bronchial asthma [Tao et al., Oxid Med Cell Longev., 2020], lung injury caused by a bacterial infection, notably by Pseudomonas aeruginosa [Dar et al., J Clin Invest., 2018, 128(10), 4639-4653] or Mycobacterium tuberculosis [Amaral et al., J Exp Med., 2019, 216(3): 556-570] and pulmonary fibrosis, such as radiation induced-lung fibrosis (RILF) [Li et al., J Inflamm., 2019, 16:11] and paraquat-induced pulmonary damage [Rashidipour et al., Toxicology, 2020, 433-434:152407].

Pathologies associated with ferroptosis affecting the bowel include necrotizing enterocolitis [Subramanian et al., Acta Physiologica Sinica, 2020, 72(3)] and inflammatory bowel diseases, such as Crohn's disease [Mayr et al., Nat Commun., 2020, 11(1)].

Pathologies associated with ferroptosis affecting the whole body include haemochromatosis [Imoto et al., Transfus Apher Sci., 2018, 57(4), 524-531], hemolytic disorders [Youssef et al., 2019, Ferroptosis in Hemolytic Disorders. In: Tang D. (eds) Ferroptosis in Health and Disease. Springer, Chain.], cytokinic storm during a viral infection [Edeas et al., Int J Infect Dis., 2020, 97; Yang and Lai, Cell Death Discov., 2020, 6], radiation-induced necrosis [Wu et al., Front Oncol., 2020, 10], rheumatoid arthritis [Xie et al., Inflammation., 2020, doi: 10.1007/s10753-020-01338-2], type I diabetes [Bruni et al., Cell Transplant., 2018, 27(6)], insulin resistance related to obesity, epilepsy, including mitochondrial disease-related epilepsy and intractable epilepsy [Kahn-Kirby et al., PLoS One., 2019, 14(3)], and pathologies related to stress-induced premature tissue senescence, such as atherosclerosis [Bai et al., Free Radic Biol Med., 2020, 160], hypertension [Yang et al., Clin Exp Hypertens., 2020, 42(8)] and type II diabetes [Li et al., Nutrients., 2020, 12(10)].

Therefore, inhibition of ferroptosis is a new and attractive therapeutic strategy for the above diseases.

There exists thus a need for new ferroptosis inhibitors with high potential, good stability and low toxicity.

The inventors have discovered new inhibitors of ferroptosis regulated cell death, which appear to be very attractive for preventing and/or treating disorders associated with ferroptosis.

Hence, the present invention relates to a compound of the following general formula (I):

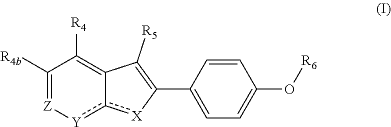

or a pharmaceutically acceptable salt and/or solvate thereof,
wherein:

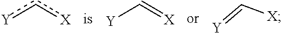

(i) when

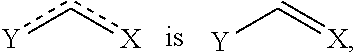

X is N, Y is N(R₂) and Z is C(H);
(ii) when

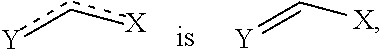

X is N(R₁), and
Y is N or N⁺(O⁻) and Z is C(R₃), or
Y is CH and Z is N, or
Y and Z are CH;
   and wherein:
R₁ and R₂ represent, independently of each other, a hydrogen atom, CN, NO₂, OR₇, SR₈, NR₉R₁₀, C(O)R₁₁, CO₂R₁₂, OC(O)R₁₃, NR₁₄C(O)R₁₅, C(O)NR₁₆R₁₇, S(O)R_{S}, SO₂R_{S}′, a (C₁-C₆)alkyl, a (C₁-C₆)haloalkyl, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, CN, NO₂, OR₁₈, SR₁₉, NR₂₀R₂₁, C(O)R₂₂, CO₂R₂₃, OC(O)R₂₄, NR₂₅C(O)R₂₆, C(O)NR₂₇R₂₈, a (C₁-C₆)alkyl and a (C₁-C₆)haloalkyl group;
R₃, R₄, R_{4b} and R₅ represent, independently of each other, a hydrogen atom, a halogen atom, CN, OR₂₉, SR₃₀, NR₃₁R₃₂, C(O)R₃₃, CO₂R₃₄, OC(O)R₃₅, NR₃₆C(O)R₃₇, C(O)NR₃₈R₃₉, a (C₁-C₆)alkyl, a (C₁-C₆)haloalkyl group, said alkyl or haloalkyl group being optionally substituted by one or more substituents selected from the group consisting of OR₄₀, SR₄₁ and NR₄₂R₄₃, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, CN, NO₂, OR₄₄, SR₄₅, NR₄₆R₄₇, C(O)R₄₈, CO₂R₄₉, OC(O)R₅₀, NR₅₁C(O)R₅₂, C(O)NR₅₃R₅₄, a (C₁-C₆)alkyl and a (C₁-C₆)haloalkyl group;
R₆ represents a hydrogen atom, a (C₁-C₆)alkyl or an aryl-(C₁-C₆)alkyl group, or a (C₁-C₆)alkylcarbonyl group optionally substituted with one or more substituents selected from the group consisting of OH, SH, NH₂, a (C₁-C₆)alkoxy, a (C₁-C₆)thioalkoxy, a (C₁-C₆)alkylamino and a di((C₁-C₆)alkyl)amino group;
R_{S} and R_{S}′ represent, independently of each other a (C₁-C₆)alkyl, an aryl or an aryl-(C₁-C₆)alkyl group;
R₇-R₁₀, R₁₂, R₁₄ and R₁₆-R₁₇ represent, independently of each other, a hydrogen atom, a (C₁-C₆)alkyl, an aryl or an aryl-(C₁-C₆)alkyl group;
R₁₁, R₁₃ and R₁₅ represent, independently of each other, a hydrogen atom, a (C₁-C₆)alkyl, an aryl, an aryl-(C₁-C₆)alkyl, a (C₁-C₆)alkoxy, a (C₁-C₆)alkylamino or a di((C₁-C₆)alkyl)amino group;
R₁₈ to R₂₈ represent, independently of each other, a hydrogen atom, a (C₁-C₆)alkyl or an aryl group;
R₂₉ to R₃₉ represent, independently of each other, a hydrogen atom, a (C₁-C₆)alkyl, an aryl or an aryl-(C₁-C₆)alkyl group, said aryl group being optionally substituted by one or more substituents selected from the group consisting of a halogen atom, CN, NO₂, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group;
R₄₀ to R₄₃ represent, independently of each other, a hydrogen atom or a (C₁-C₆)alkyl group;
R₄₄ to R₅₄ represent, independently of each other, a hydrogen atom, a (C₁-C₆)alkyl or an aryl group; and
R₅₅ to R₆₅ represent, independently of each other, a hydrogen atom, a (C₁-C₆)alkyl or an aryl group;
   for use as a drug for inhibiting ferroptosis.

For the purpose of the invention, the term “pharmaceutically acceptable” is intended to mean what is useful to the preparation of a pharmaceutical composition, and what is generally safe and non-toxic, for a pharmaceutical use.

The term “pharmaceutically acceptable salt or solvate” is intended to mean, in the framework of the present invention, a salt or solvate of a compound which is pharmaceutically acceptable, as defined above, and which possesses the pharmacological activity of the corresponding compound.

The pharmaceutically acceptable salts comprise:
(1) acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic, and trifluoroacetic acid and the like, and
(2) base addition salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate and sodium hydroxide.

Acceptable solvates for the therapeutic use of the compounds of the present invention include conventional solvates such as those formed during the last step of the preparation of the compounds of the invention due to the presence of solvents.

The term “halogen”, as used in the present invention, refers to a fluorine, bromine, chlorine or iodine atom.

The terms “(C₁-C₆)alkyl”, as used in the present invention, refers to a straight or branched saturated hydrocarbon chain containing from 1 to 6 carbon atoms including, but not limited to, methyl, ethyl, n-propyl, iso-propyl, n-butyl, iso-butyl, sec-butyl, t-butyl, n-pentyl, n-hexyl, and the like.

The term “(C₁-C₆)haloalkyl”, as used in the present invention, refers to a (C₁-C₆)alkyl group as defined above in which part or all of the hydrogen atoms is replaced with a halogen atom as defined above. This means that the (C₁-C₆)alkyl group is substituted by at least one halogen atom. It can be for example a trifluoromethyl group.

The term “aryl”, as used in the present invention, refers to an aromatic hydrocarbon group comprising preferably 6 to 10 carbon atoms and comprising one or more, notably 1 or 2, fused rings, such as, for example, a phenyl or naphtyl group, advantageously a phenyl group.

The term “heterocyclic” as used in the present invention refers to a saturated, unsaturated (i.e. not aromatic) or aromatic monocyclic or bicyclic group comprising two fused, bridged or spiro rings, preferably fused rings, advantageously comprising 5 to 10, notably 5 or 6, atoms in each ring, in which the atoms of the ring(s) comprise one or more, advantageously 1 to 3, heteroatoms selected from O, S and N, preferably O and N, the remainder being carbon atoms.

A saturated heterocyclic group is more particularly a 5- or 6-membered saturated monocyclic heterocyclic group such as a pyrrolidinyl, tetrahydrofuranyl, tetrahydrothiophenyl, thiazolidinyl, isothiazolidinyl, oxazolidinyl, isoxazolidinyl, imidazolidinyl, pyrazolidinyl, triazolidinyl, piperidinyl, piperazinyl, morpholinyl or thiomorpholinyl group.

An unsaturated heterocyclic group is more particularly an unsaturated monocyclic or bicyclic heterocyclic group, each cycle comprising 5 or 6 members, such as a pyrrolinyl, dihydrofuranyl, dihydrothiophenyl, thiazolinyl, isothiazolinyl, oxazolinyl, isoxazolinyl, imidazolinyl, pyrazolinyl, triazolinyl, dihydropyridinyl, tetrahydropyridinyl, dihydropyrimidinyl, tetrahydropyrimidinyl, dihydropyridazinyl, tetrahydropyridazinyl, dihydropyrazinyl, tetrahydropyrazinyl, dihydrotriazinyl, tetrahydrotriazinyl, indolinyl, 2,3-dihydrobenzofuranyl, 2,3-dihydrobenzothiophenyl, 1,3-benzodioxolyl, 1,3-benzoxathiolyl, benzoxazolinyl, benzothiazolinyl, benzimidazolinyl, chromanyl or chromenyl group.

An aromatic heterocyclic group, also called heteroaryl group, is more particularly an aromatic monocyclic or bicyclic heterocyclic group, each cycle comprising 5 or 6 members, such as a pyrrolyl, furanyl, thiophenyl, thiazolyl, isothiazolyl, oxazolyl, isoxazolyl, imidazolyl, pyrazolyl, triazolyl, pyridinyl, pyrimidinyl, pyridazinyl, pyrazinyl, triazinyl (such as 1,3,5-triazinyl), indolyl, benzofuranyl, benzothiophenyl, benzothiazolyl, benzoxazolyl, benzimidazolyl, indazolyl, benzotriazolyl, purinyl, quinolinyl, isoquinolinyl, cinnolinyl, quinazolinyl or quinoxalinyl group.

The term “aryl-(C₁-C₆)alkyl”, as used in the present invention, refers to an aryl group as defined above bound to the molecule via a (C₁-C₆)alkyl group as defined above. In particular, the —(C₁-C₆)alkyl-aryl group is a benzyl group.

The term “heterocyclyl-(C₁-C₆)alkyl”, as used in the present invention, refers to a heterocyclyl group as defined above bound to the molecule via a (C₁-C₆)alkyl group as defined above. In particular, the —(C₁-C₆)alkyl-heterocyclyl group is 5- or 6-membered saturated monocyclic heterocyclic group as defined above bound to the molecule via a (C₁-C₆)alkyl group as defined above.

The term “(C₁-C₆)alkylcarbonyl”, as used in the present invention, refers to a (C₁-C₆)alkyl group as defined above bound to the molecule via a —C(═O)— group, including, but not limited to, acetyl, propionyl, butanoyl, pentanoyl, hexanoyl and the like.

The term “(C₁-C₆)alkoxy”, as used in the present invention, refers to a (C₁-C₆)alkyl group as defined above bound to the molecule via an oxygen atom, including, but not limited to, methoxy, ethoxy, n-propoxy, iso-propoxy, n-butoxy, iso-butoxy, sec-butoxy, t-butoxy, n-pentoxy, n-hexoxy, and the like.

The term “(C₁-C₆)thioalkoxy”, as used in the present invention, refers to a (C₁-C₆)alkyl group as defined above bound to the molecule via a sulfur atom, including, but not limited to, thiomethoxy, thioethoxy, n-thiopropoxy, iso-thiopropoxy, n-thiobutoxy, iso-thiobutoxy, sec-thiobutoxy, t-thiobutoxy, n-thiopentoxy, n-thiohexoxy, and the like.

The term “(C₁-C₆)alkylamino”, as used in the present invention, refers to a -NHAlk group with Alk representing a (C₁-C₆)alkyl group as defined above, including, but not limited to, methylamino, ethylamino, n-propylamino, iso-propylamino, n-butylamino, iso-butylamino, sec-butylamino, t-butylamino, n-pentylamino, n-hexylamino, and the like.

The term “di(C₁-C₆)alkylamino”, as used in the present invention, refers to a —NAlk₁Alk₂ group with Alk₁ and Alk₂ representing, independently of one another, a (C₁-C₆)alkyl group as defined above, including, but not limited to, dimethylamino, diethylamino, ethylmethylamino and the like.

According to a particular embodiment of the present invention, R₁ represents a hydrogen atom, CN, NO₂, OR₇, SR₈, NR₉R₁₀, C(O)R₁₁, CO₂R₁₂, OC(O)R₁, NR₁₄C(O)R₁₅, C(O)NR₁₆R₁₇, S(O)R_{S}, SO₂R_{S}′, a (C₁-C₆)alkyl, a (C₁-C₆)haloalkyl, an aryl, a heterocyclyl, an aryl-(C₁-C₆)alkyl or a heterocyclyl-(C₁-C₆)alkyl group, wherein said aryl or heterocyclyl group (which may be part of a aryl-(C₁-C₆)alkyl or heterocyclyl-(C₁-C₆)alkyl group) is optionally substituted by one or more substituents, notably one susbtituent, selected from the group consisting of a halogen atom, CN, NO₂, OR₁₈, SR₁₉, NR₂₀R₂₁, a (C₁-C₆)alkyl and a (C₁-C₆)haloalkyl, notably a halogen atom, NO₂, OR₁₈, a (C₁-C₆)alkyl and a (C₁-C₆)haloalkyl group, in particular NO₂ and OR₁₈, and wherein R₁₈ to R₂₁ represent, independently of each other, a hydrogen atom or a (C₁-C₆)alkyl group.

According to another particular embodiment of the present invention, R₁ represents a hydrogen atom, CN, NO₂, OR₇, SR₈, NR₉R₁₀, C(O)R₁₁, CO₂R₁₂, OC(O)R₁, NR₁₄C(O)R₁₅, C(O)NR₁₆R₁₇, a (C₁-C₆)alkyl, a (C₁-C₆)haloalkyl, an aryl, a heterocyclyl, an aryl-(C₁-C₆)alkyl or a heterocyclyl-(C₁-C₆)alkyl group, wherein said aryl or heterocyclyl group is optionally substituted by one or more substituents as defined above.

According to still another particular embodiment of the present invention, R₁ represents a hydrogen atom, CN, OR₇, C(O)R₁₁, CO₂R₁₂, OC(O)R₁₃, SO₂R_{S}′, a (C₁-C₆)alkyl, a heterocyclyl or a heterocyclyl-(C₁-C₆)alkyl group, wherein said heterocyclyl group (which may be part of a heterocyclyl-(C₁-C₆)alkyl group) is optionally substituted by one or more substituents, notably one substituent, selected from the group consisting of a halogen atom, CN, NO₂, OR₁₈, SR₁₉, NR₂₀R₂₁, a (C₁-C₆)alkyl and a (C₁-C₆)haloalkyl group, notably a halogen atom, NO₂, OR₁₈, a (C₁-C₆)alkyl and a (C₁-C₆)haloalkyl group, in particular NO₂ and OR₁₈, preferably said heterocyclyl group is optionally substituted by NO₂, and wherein R₁₈ to R₂₁ represent, independently of each other, a hydrogen atom or a (C₁-C₆)alkyl group.

According to yet another particular embodiment of the present invention, R₁ represents a hydrogen atom, CN, OR₇, C(O)R₁₁, CO₂R₁₂, OC(O)R₁₃, a heterocyclyl or a heterocyclyl-(C₁-C₆)alkyl group, wherein said heterocyclyl group is optionally substituted by one or more substituents as defined above.

In the above embodiments, the (C₁-C₆)alkyl group, which may be part of an aryl-(C₁-C₆)alkyl group or a heterocyclyl-(C₁-C₆)alkyl group, is preferably a (C₁-C₃)alkyl group.

In the above embodiments, the aryl group, which may be part of an aryl-(C₁-C₆)alkyl group, is preferably a phenyl group.

In the above embodiments, the heterocyclyl group, which may be part of a heterocyclyl-(C₁-C₆)alkyl group, is in particular a 5- or 6-membered, saturated, unsaturated (i.e. not aromatic) or aromatic, notably saturated or aromatic, monocyclic group, in which the atoms of the ring comprise one or more, advantageously 1 to 3, heteroatoms selected from O, S and N, preferably O and N, the remainder being carbon atoms, such as a morpholinyl, a pyridinyl or a piperazinyl, for instance a morpholinyl or pyridinyl group.

In the above embodiments, R_{S} and R_{S}′ represent, independently of each other a (C₁-C₆)alkyl, an aryl or an aryl-(C₁-C₆)alkyl group, notably a (C₁-C₆)alkyl or an aryl group, in particular, an aryl group, such as a phenyl group.

In the above embodiments, R₇-R₁₀, R₁₂, R₁₄ and R₁₆-R₁₇ represent, independently of each other, a hydrogen atom, a (C₁-C₆)alkyl, an aryl or an aryl-(C₁-C₆)alkyl group, and R₁₁, R₁₃ and R₁₅ represent, independently of each other, a hydrogen atom, a (C₁-C₆)alkyl, an aryl, an aryl-(C₁-C₆)alkyl, a (C₁-C₆)alkoxy, a (C₁-C₆)alkylamino or a di((C₁-C₆)alkyl)amino group, notably a hydrogen atom, a (C₁-C₆)alkyl, an aryl, a (C₁-C₆)alkylamino or a di((C₁-C₆)alkyl)amino group, in particular a (C₁-C₃)alkyl, an aryl such as a phenyl, a (C₁-C₃)alkylamino or a di((C₁-C₃)alkyl)amino group.

In particular, in the above embodiments, R₇ to R₁₇ represent, independently of each other, a hydrogen atom, a (C₁-C₆)alkyl, an aryl or an aryl-(C₁-C₆)alkyl group, notably a hydrogen atom, a (C₁-C₆)alkyl or an aryl group, typically a hydrogen atom, a (C₁-C₃)alkyl or an aryl group, wherein the aryl group, which may be part of an aryl-(C₁-C₆)alkyl group, is preferably a phenyl group.

According to a particular embodiment of the present invention, R₂ represents a hydrogen atom, CN, NO₂, OR₇, SR₈, NR₉R₁₀, C(O)R₁₁, CO₂R₁₂, OC(O)R₁, NR₁₄C(O)R₁₅, C(O)NR₁₆R₁₇, S(O)R_{S}, SO₂R_{S}′, a (C₁-C₆)alkyl, a (C₁-C₆)haloalkyl, an aryl, a heterocyclyl, an aryl-(C₁-C₆)alkyl or a heterocyclyl-(C₁-C₆)alkyl group, wherein said aryl or heterocyclyl group (which may be part of a aryl-(C₁-C₆)alkyl or heterocyclyl-(C₁-C₆)alkyl group) is optionally substituted by one or more substituents, notably by one susbtituent, selected from the group consisting of a halogen atom, CN, NO₂, OR₁₈, SR₁₉, NR₂₀R₂₁, a (C₁-C₆)alkyl and a (C₁-C₆)haloalkyl, notably a halogen atom, NO₂, OR₁₈, a (C₁-C₆)alkyl and a (C₁-C₆)haloalkyl group, in particular NO₂ and OR₁₈, and wherein R₁₈ to R₂₁ represent, independently of each other, a hydrogen atom or a (C₁-C₆)alkyl group.

In the above embodiment, R_{S} and R_{S}′ represent, independently of each other a (C₁-C₆)alkyl, an aryl or an aryl-(C₁-C₆)alkyl group, notably a (C₁-C₆)alkyl or an aryl group, in particular, an aryl group, such as a phenyl group.

According to another particular embodiment of the present invention, R₂ represents a hydrogen atom, CN, NO₂, OR₇, SR₈, NR₉R₁₀, C(O)R₁₁, CO₂R₁₂, OC(O)R₁₃, NR₁₄C(O)R₁₅, C(O)NR₁₆R₁₇, a (C₁-C₆)alkyl, a (C₁-C₆)haloalkyl, an aryl, a heterocyclyl, an aryl-(C₁-C₆)alkyl or a heterocyclyl-(C₁-C₆)alkyl group, wherein said aryl or heterocyclyl group is optionally substituted by one or more substituents as defined above.

According to still another particular embodiment of the present invention, R₂ represents C(O)R₁₁, CO₂R₁₂, C(O)NR₁₆R₁₇, a (C₁-C₆)alkyl, a (C₁-C₆)haloalkyl, an aryl, a heterocyclyl, an aryl-(C₁-C₆)alkyl or a heterocyclyl-(C₁-C₆)alkyl group, wherein said aryl or heterocyclyl group is optionally substituted by one or more substituents, notably one substituent, selected from the group consisting of a halogen atom OR₁₈, SR₁₉, NR₂₀R₂₁, a (C₁-C₆)alkyl and a (C₁-C₆)haloalkyl group, and wherein R₁₈ to Rn represent, independently of each other, a hydrogen atom or a (C₁-C₆)alkyl group.

According to yet another particular embodiment of the present invention, R₂ represents CO₂R₁₂, C(O)NR₁₆R₁₇, a (C₁-C₆)alkyl, an aryl or an aryl-(C₁-C₆)alkyl group, notably CO₂R₁₂, C(O)NR₁₆R₁₇ or an aryl-(C₁-C₆)alkyl group, wherein said aryl group is optionally substituted by one or more substituents, notably one substituent, selected from the group consisting of a halogen atom, OR₁₈, SR₁₉ and NR₂₀R₂₁, notably OR₁₈, and wherein R₁₈ to R₂₁ represent, independently of each other, a hydrogen atom or a (C₁-C₆)alkyl group.

In the above embodiments, the (C₁-C₆)alkyl group, which may be part of an aryl-(C₁-C₆)alkyl group or a heterocyclyl-(C₁-C₆)alkyl group, is preferably a (C₁-C₃)alkyl group.

In the above embodiments, the aryl group, which may be part of an aryl-(C₁-C₆)alkyl group, is preferably a phenyl group.

In the above embodiments, the heterocyclyl group, which may be part of a heterocyclyl-(C₁-C₆)alkyl group, is in particular a 5- or 6-membered, saturated, unsaturated (i.e. not aromatic) or aromatic, notably saturated, monocyclic group, in which the atoms of the ring comprise one or more, advantageously 1 to 3, heteroatoms selected from O, S and N, preferably O and N, the remainder being carbon atoms, such as a morpholinyl, a pyridinyl or a piperazinyl group, notably a piperazinyl group.

In the above embodiments, R₇-R₁₀, R₁₂, R₁₄ and R₁₆-R₁₇ represent, independently of each other, a hydrogen atom, a (C₁-C₆)alkyl, an aryl or an aryl-(C₁-C₆)alkyl group, and R₁₁, R₁₃ and R₁₅ represent, independently of each other, a hydrogen atom, a (C₁-C₆)alkyl, an aryl, an aryl-(C₁-C₆)alkyl, a (C₁-C₆)alkoxy, a (C₁-C₆)alkylamino or a di((C₁-C₆)alkyl)amino group, notably a hydrogen atom, a (C₁-C₆)alkyl, an aryl, a (C₁-C₆)alkylamino or a di((C₁-C₆)alkyl)amino group, in particular a (C₁-C₃)alkyl, an aryl such as a phenyl, a (C₁-C₃)alkylamino or a di((C₁-C₃)alkyl)amino group.

In particular, in the above embodiments, R₇ to R₁₇ represent, independently of each other, a hydrogen atom, a (C₁-C₆)alkyl, an aryl or an aryl-(C₁-C₆)alkyl group, notably a hydrogen atom, a (C₁-C₆)alkyl or an aryl-(C₁-C₆)alkyl group, typically a hydrogen atom, a (C₁-C₃)alkyl or an aryl group, wherein the aryl group, which may be part of an aryl-(C₁-C₆)alkyl group, is preferably a phenyl group.

According to another particular embodiment of the present invention, R₃ represents a hydrogen atom, a halogen atom, CN, OR₂₉, SR₃₀, NR₃₁R₃₂, C(O)R₃₃, CO₂R₃₄, OC(O)R₃₅, NR₃₆C(O)R₃₇, C(O)NR₃₈R₃₉, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, OR₄₄, SR₄₅, NR₄₆R₄₇, a (C₁-C₆)alkyl and a (C₁-C₆)haloalkyl group, and wherein R₂₉ to R₃₉ represent, independently of each other, a hydrogen atom, a (C₁-C₆)alkyl, an aryl, or an aryl-(C₁-C₆)alkyl group, notably a hydrogen atom or a (C₁-C₆)alkyl group, typically a hydrogen atom or a (C₁-C₃)alkyl group.

According to another particular embodiment of the present invention, R₃ represents a hydrogen atom, a halogen atom, CN, OR₂₉, SR₃₀, NR₃₁R₃₂, C(O)R₃₃, CO₂R₃₄, OC(O)R₃₅, NR₃₆C(O)R₃₇ or C(O)NR₃₈R₃₉, wherein R₂₉ to R₃₉ represent, independently of each other, a hydrogen atom, a (C₁-C₆)alkyl, an aryl, or an aryl-(C₁-C₆)alkyl group, notably a hydrogen atom or a (C₁-C₆)alkyl group.

According to still another particular embodiment of the present invention, R₃ represents a hydrogen atom, a halogen atom, CN, OR₂₉, SR₃₀, NR₃₁R₃₂, OC(O)R₃₅, NR₃₆C(O)R₃₇, a heterocyclyl or a heterocyclyl-(C₁-C₆)alkyl group, wherein said heterocyclyl group is optionally substituted by one or more substituents selected from the group consisting of a halogen atom, OR₄₄, SR₄₅, NR₄₆R₄₇ and a (C₁-C₆)alkyl a group, preferably, R₃ represents a hydrogen atom, a halogen atom, CN, NR₃₁R₃₂, OC(O)R₃₅, a heterocyclyl or a heterocyclyl-(C₁-C₆)alkyl group, wherein said heterocyclyl group is optionally substituted by one or more substituents selected from the group consisting of a OR₄₄, SR₄₅ and NR₄₆R₄₇, notably OR₄₄, and wherein R₂₉ to R₃₇ represent, independently of each other, a hydrogen atom, a (C₁-C₆)alkyl, an aryl, or an aryl-(C₁-C₆)alkyl group, notably a hydrogen atom or a (C₁-C₆)alkyl group, typically a hydrogen atom or a (C₁-C₃)alkyl group.

In the above embodiments, R₄₄ to R₄₇ represent, independently of each other, a hydrogen atom or a (C₁-C₆)alkyl group, notably a hydrogen atom or a (C₁-C₃)alkyl group.

According to yet another particular embodiment of the present invention, R₃ represents a hydrogen atom, a halogen atom, OR₂₉, SR₃₀, NR₃₁R₃₂, C(O)R₃₃, CO₂R₃₄, OC(O)R₃₅, NR₃₆C(O)R₃₇ or C(O)NR₃₈R₃₉, preferably a hydrogen atom, a halogen atom, OR₂₉, SR₃₀, NR₃₁R₃₂, OC(O)R₃₅ or NR₃₆C(O)R₃₇, more preferably a hydrogen atom or OC(O)R₃₅, wherein R₂₉ to R₃₇ represent, independently of each other, a hydrogen atom, a (C₁-C₆)alkyl, an aryl, or an aryl-(C₁-C₆)alkyl group, notably a hydrogen atom or a (C₁-C₆)alkyl group, typically a hydrogen atom or a (C₁-C₃)alkyl group.

In the above embodiments, the aryl group, which may be part of an aryl-(C₁-C₆)alkyl group, is preferably a phenyl group.

In the above embodiments, the heterocyclyl group, which may be part of a heterocyclyl-(C₁-C₆)alkyl group, is in particular a 5- or 6-membered, saturated, unsaturated (i.e. not aromatic) or aromatic, notably aromatic, monocyclic group, in which the atoms of the ring comprise one or more, advantageously 1 to 3, heteroatoms selected from O, S and N, preferably O and N, the remainder being carbon atoms, such as a pyridinyl, a pyrimidinyl, a pyrazolyl, a piperazinyl or a piperidinyl group, for instance a pyridinyl, a pyrimidinyl or a pyrazolyl group.

In the above embodiments, the (C₁-C₆)alkyl group, which may be part of an aryl-(C₁-C₆)alkyl group or a heterocyclyl-(C₁-C₆)alkyl group, is preferably a (C₁-C₃)alkyl group.

According to a particular embodiment of the present invention, R₄ represents a hydrogen atom, a halogen atom, CN, OR₂₉, SR₃₀, NR₃₁R₃₂, a (C₁-C₆)alkyl, a (C₁-C₆)haloalkyl group, an aryl, a heterocyclyl, an aryl-(C₁-C₆)alkyl or a heterocyclyl-(C₁-C₆)alkyl group, wherein said aryl or heterocyclyl group (which may be part of a aryl-(C₁-C₆)alkyl or heterocyclyl-(C₁-C₆)alkyl group) is optionally substituted by one or more substituents, notably one substituent, selected from the group consisting of a halogen atom, CN, NO₂, OR₄₄, SR₄₅, NR₄₆R₄₇, C(O)R₄₈, CO₂R₄₉, OC(O)R₅₀, NR₅₁C(O)R₅₂, C(O)NR₅₃R₅₄, a (C₁-C₆)alkyl and a (C₁-C₆)haloalkyl group, notably a halogen atom, OR₄₄, SR₄₅, NR₄₆R₄₇, C(O)R₄₈, CO₂R₄₉, C(O)NR₅₃R₅₄, a (C₁-C₆)alkyl and a (C₁-C₆)haloalkyl group, in particular C(O)R₄₈, CO₂R₄₉, C(O)NR₅₃R₅₄, a (C₁-C₆)alkyl and a (C₁-C₆)haloalkyl group, preferably C(O)R₄₈ and a (C₁-C₆)alkyl group.

According to another particular embodiment of the present invention, R₄ represents a hydrogen atom, a halogen atom, CN, OR₂₉, SR₃₀, NR₃₁R₃₂, a (C₁-C₆)alkyl, a (C₁-C₆)haloalkyl group, an aryl, a heterocyclyl, an aryl-(C₁-C₆)alkyl or a heterocyclyl-(C₁-C₆)alkyl group, wherein said aryl or heterocyclyl group is optionally substituted by one or more substituents as defined above.

According to still another particular embodiment of the present invention, R₄ represents a hydrogen atom, a halogen atom, OR₂₉, SR₃₀, NR₃₁R₃₂, an aryl, or a heterocyclyl group, wherein said aryl or heterocyclyl group is optionally substituted by one or more substituents, notably one substituent, selected from the group consisting of a halogen atom, OR₄₄, SR₄₅, NR₄₆R₄₇, C(O)R₄₈, CO₂R₄₉, C(O)NR₅₃R₅₄, a (C₁-C₆)alkyl and a (C₁-C₆)haloalkyl group, in particular C(O)R₄₈, CO₂R₄₉, C(O)NR₅₃R₅₄, a (C₁-C₆)alkyl and a (C₁-C₆)haloalkyl group, preferably C(O)R₄₈ and a (C₁-C₆)alkyl group.

According to yet another particular embodiment of the present invention, R₄ represents a hydrogen atom, a halogen atom, NR₃₁R₃₂, an aryl or a heterocyclyl group, wherein said aryl or heterocyclyl group is optionally substituted by one or more substituents, notably one substituent, selected from the group consisting of C(O)R₄₈, CO₂R₄₉, C(O)NR₅₃R₅₄, a (C₁-C₆)alkyl and a (C₁-C₆)haloalkyl group, preferably C(O)R₄₈ and a (C₁-C₆)alkyl group.

In the above embodiments, the aryl group, which may be part of an aryl-(C₁-C₆)alkyl group, is preferably a phenyl group.

In the above embodiments, the heterocyclyl group, which may be part of a heterocyclyl-(C₁-C₆)alkyl group, is in particular a 5- or 6-membered, saturated, unsaturated (i.e. not aromatic) or aromatic, notably saturated, monocyclic group, in which the atoms of the ring comprise one or more, advantageously 1 to 3, heteroatoms selected from O, S and N, preferably O and N, the remainder being carbon atoms, such as a piperazinyl, a piperidinyl, a pyridinyl, a pyrimidinyl or a pyrazolyl group, for instance a piperazinyl or a piperidinyl group.

In the above embodiments, R₂₉ to R₃₂ represent, independently of each other, a hydrogen atom, a (C₁-C₆)alkyl, an aryl or an aryl-(C₁-C₆)alkyl group, said aryl group, which may be part of an aryl-(C₁-C₆)alkyl group, being preferably a phenyl and being optionally substituted by one or more substituents, notably one substituent, selected from the group consisting of a halogen atom, CN, NO₂, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group, advantageously a halogen atom, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group, notably C(O)R₅₉, CO₂R₆₀, C(O)NR₆₄R₆₅, a (C₁-C₆)alkyl and a (C₁-C₆)haloalkyl group, in particular C(O)R₅₉, wherein R₅₅ to R₆₅ represent, independently of each other, a hydrogen atom, a (C₁-C₆)alkyl or an aryl group, notably an aryl group, preferably a phenyl group.

In the above embodiments, R₄₄ to R₅₄ represent, independently of each other, a hydrogen atom, a (C₁-C₆)alkyl or an aryl group, notably an aryl group, preferably a phenyl group.

In the above embodiments, the (C₁-C₆)alkyl group, which may be part of an aryl-(C₁-C₆)alkyl group or a heterocyclyl-(C₁-C₆)alkyl group, is preferably a (C₁-C₃)alkyl group.

According to a particular embodiment of the present invention, R_{4b} represents a hydrogen atom, a halogen atom, OR₂₉, SR₃₀, NR₃₁R₃₂, a (C₁-C₆)alkyl, a (C₁-C₆)haloalkyl group, an aryl, a heterocyclyl, an aryl-(C₁-C₆)alkyl or a heterocyclyl-(C₁-C₆)alkyl group, wherein said aryl or heterocyclyl group (which may be part of a aryl-(C₁-C₆)alkyl or heterocyclyl-(C₁-C₆)alkyl group) is optionally substituted by one or more substituents, notably one substituent, selected from the group consisting of a halogen atom, CN, NO₂, OR₄₄, SR₄₅, NR₄₆R₄₇, C(O)R₄₈, CO₂R₄₉, OC(O)R₅₀, NR₅₁C(O)R₅₂, C(O)NR₅₃R₅₄, a (C₁-C₆)alkyl and a (C₁-C₆)haloalkyl group, notably a halogen atom, OR₄₄, SR₄₅, NR₄₆R₄₇, C(O)R₄₈, CO₂R₄₉, C(O)NR₅₃R₅₄, a (C₁-C₆)alkyl and a (C₁-C₆)haloalkyl group, in particular C(O)R₄₈, CO₂R₄₉, C(O)NR₅₃R₅₄, a (C₁-C₆)alkyl and a (C₁-C₆)haloalkyl group, preferably C(O)R₄₈ and a (C₁-C₆)alkyl group.

According to a particular embodiment of the present invention, R_{4b} represents a hydrogen atom, a halogen atom, OR₂₉, SR₃₀, NR₃₁R₃₂, an aryl, or a heterocyclyl group, wherein said aryl or heterocyclyl group is optionally substituted by one or more substituents, notably one substituent, selected from the group consisting of a halogen atom, OR₄₄, SR₄₅, NR₄₆R₄₇, C(O)R₄₈, CO₂R₄₉, C(O)NR₅₃R₅₄, a (C₁-C₆)alkyl and a (C₁-C₆)haloalkyl group, in particular C(O)R₄₈, CO₂R₄₉, C(O)NR₅₃R₅₄, a (C₁-C₆)alkyl and a (C₁-C₆)haloalkyl group, preferably C(O)R₄₈ and a (C₁-C₆)alkyl group.

According to yet another particular embodiment of the present invention, R_{4b} represents a hydrogen atom, a halogen atom, NR₃₁R₃₂, an aryl or a heterocyclyl group, wherein said aryl or heterocyclyl group is optionally substituted by one or more substituents, notably one substituent, selected from the group consisting of C(O)R₄₈, CO₂R₄₉, C(O)NR₅₃R₅₄, a (C₁-C₆)alkyl and a (C₁-C₆)haloalkyl group, preferably C(O)R₄₈ and a (C₁-C₆)alkyl group.

According to still another particular embodiment of the present invention, R_{4b} represents a hydrogen atom, a halogen atom, OR₂₉ or NR₃₁R₃₂, preferably a hydrogen atom, a halogen atom or OR₂₉, more preferably a hydrogen atom.

In the above embodiments, the aryl group, which may be part of an aryl-(C₁-C₆)alkyl group, is preferably a phenyl group.

In the above embodiments, the heterocyclyl group, which may be part of a heterocyclyl-(C₁-C₆)alkyl group, is in particular a 5- or 6-membered, saturated, unsaturated (i.e. not aromatic) or aromatic, notably saturated, monocyclic group, in which the atoms of the ring comprise one or more, advantageously 1 to 3, heteroatoms selected from O, S and N, preferably O and N, the remainder being carbon atoms.

In the above embodiments, R₂₉ to R₃₂ represent, independently of each other, a hydrogen atom, a (C₁-C₆)alkyl, an aryl or an aryl-(C₁-C₆)alkyl group, said aryl group, which may be part of an aryl-(C₁-C₆)alkyl group, being preferably a phenyl and being optionally substituted by one or more substituents, notably one substituent, selected from the group consisting of a halogen atom, CN, NO₂, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group, advantageously a halogen atom, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group, notably C(O)R₅₉, CO₂R₆₀, C(O)NR₆₄R₆₅, a (C₁-C₆)alkyl and a (C₁-C₆)haloalkyl group, in particular C(O)R₅₉, wherein R₅₅ to R₆₅ represent, independently of each other, a hydrogen atom, a (C₁-C₆)alkyl or an aryl group, notably an aryl group, preferably a phenyl group. Preferably, R₂₉ to R₃₂ represent, independently of each other, a hydrogen atom or a (C₁-C₆)alkyl group, notably a hydrogen atom.

In the above embodiments, R₄₄ to R₅₄ represent, independently of each other, a hydrogen atom, a (C₁-C₆)alkyl or an aryl group, notably an aryl group, preferably a phenyl group.

In the above embodiments, the (C₁-C₆)alkyl group, which may be part of an aryl-(C₁-C₆)alkyl group or a heterocyclyl-(C₁-C₆)alkyl group, is preferably a (C₁-C₃)alkyl group.

In a particular embodiment of the present invention, R₄ is as defined above and R_{4b} represents a hydrogen atom, a halogen atom, OR₂₉ or NR₃₁R₃₂, wherein R₂₉ to R₃₂ represent, independently of each other, a hydrogen atom or a (C₁-C₆)alkyl group, notably a hydrogen atom, preferably R_{4b} represents a hydrogen atom, a halogen atom or OR₂₉, more preferably a hydrogen atom.

In a particular embodiment of the present invention, R₅ represents a hydrogen atom, a halogen atom, CN, OR₂₉, SR₃₀, NR₃₁R₃₂, a (C₁-C₆)alkyl, a (C₁-C₆)haloalkyl group, said alkyl or haloalkyl group being optionally substituted by one or more substituents selected from the group consisting of OR₄₀, SR₄₁ and NR₄₂R₄₃, an aryl, a heterocyclyl, an aryl-(C₁-C₆)alkyl or a heterocyclyl-(C₁-C₆)alkyl group, wherein said aryl or heterocyclyl group (which may be part of a aryl-(C₁-C₆)alkyl or heterocyclyl-(C₁-C₆)alkyl group) is optionally substituted by one or more substituents, notably one substituent, selected from the group consisting of a halogen atom, CN, NO₂, OR₄₄, SR₄₅, NR₄₆R₄₇, C(O)R₄₈, CO₂R₄₉, OC(O)R₅₀, NR₅₁C(O)R₅₂, C(O)NR₅₃R₅₄, a (C₁-C₆)alkyl and a (C₁-C₆)haloalkyl group, notably a halogen atom, OR₄₄, SR₄₅, NR₄₆R₄₇, C(O)R₄₈, CO₂R₄₉, C(O)NR₅₃R₅₄, a (C₁-C₆)alkyl and a (C₁-C₆)haloalkyl group, in particular a halogen atom, OR₄₄, SR₄₅, NR₄₆R₄₇, a (C₁-C₆)alkyl and a (C₁-C₆)haloalkyl group, notably a halogen atom, a (C₁-C₆)alkyl and a (C₁-C₆)haloalkyl group, preferably a (C₁-C₆)alkyl group.

In the above embodiment, R₂₉ to R₃₂ represent, independently of each other, a hydrogen atom, a (C₁-C₆)alkyl, an aryl or an aryl-(C₁-C₆)alkyl group, said aryl group, which may be part of an aryl-(C₁-C₆)alkyl group, being preferably a phenyl and being optionally substituted by one or more substituents, notably one substituent, selected from the group consisting of a halogen atom, CN, NO₂, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group, advantageously a halogen atom, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group, notably C(O)R₅₉, CO₂R₆₀, C(O)NR₆₄R₆₅, a (C₁-C₆)alkyl and a (C₁-C₆)haloalkyl group, in particular C(O)R₅₉, wherein R₅₅ to R₆₅ represent, independently of each other, a hydrogen atom, a (C₁-C₆)alkyl or an aryl group, notably an aryl group, preferably a phenyl group. Preferably, R₂₉ to R₃₂ represent, independently of each other, independently of each other, a hydrogen atom or a (C₁-C₆)alkyl group.

In another particular embodiment of the present invention, R₅ represents a hydrogen atom, a halogen atom, a (C₁-C₆)alkyl, a (C₁-C₆)haloalkyl group, said alkyl or haloalkyl group being optionally substituted by one or more substituents, notably one substituent, selected from the group consisting of OR₄₀, SR₄₁ and NR₄₂R₄₃, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, OR₄₄, SR₄₅, NR₄₆R₄₇, a (C₁-C₆)alkyl and a (C₁-C₆)haloalkyl group, notably a halogen atom, a (C₁-C₆)alkyl and a (C₁-C₆)haloalkyl group, preferably a (C₁-C₆)alkyl group.

In yet another particular embodiment of the present invention, R₅ represents a hydrogen atom, a halogen atom, a (C₁-C₆)alkyl, a (C₁-C₆)haloalkyl group or a heterocyclyl-(C₁-C₆)alkyl group, said alkyl or haloalkyl group being optionally substituted by OR₄₀, and said heterocyclyl being optionally substituted by one or more (C₁-C₆)alkyl group.

Preferably, R₅ represents a hydrogen atom.

In the above embodiments, the aryl group, which may be part of an aryl-(C₁-C₆)alkyl group, is preferably a phenyl group.

In the above embodiments, the heterocyclyl group, which may be part of a heterocyclyl-(C₁-C₆)alkyl group, is in particular a 5- or 6-membered, saturated, unsaturated (i.e. not aromatic) or aromatic, notably saturated, monocyclic group, in which the atoms of the ring comprise one or more, advantageously 1 to 3, heteroatoms selected from O, S and N, preferably O and N, the remainder being carbon atoms, such as a piperazinyl group.

In the above embodiments, R₄₀ to R₄₃ represent, independently of each other, a hydrogen atom or a (C₁-C₆)alkyl group, notably a hydrogen atom.

In the above embodiments, R₄₄ to R₅₄ represent, independently of each other, a hydrogen atom, a (C₁-C₆)alkyl or an aryl group, notably an aryl group, preferably a phenyl group.

In the above embodiments, the (C₁-C₆)alkyl group, which may be part of an aryl-(C₁-C₆)alkyl group or a heterocyclyl-(C₁-C₆)alkyl group, is preferably a (C₁-C₃)alkyl group.

In a particular embodiment of the present invention, R₆ represents a hydrogen atom, a (C₁-C₃)alkyl or an aryl-(C₁-C₃)alkyl group, or a (C₁-C₆)alkylcarbonyl group optionally substituted with one or more substituents selected from the group consisting of OH, SH, NH₂, a (C₁-C₃)alkoxy, a (C₁-C₃)thioalkoxy and a (C₁-C₃)alkylamino group, preferably R₆ represents a hydrogen atom, a methyl, an ethyl, a benzyl or a (C₁-C₆)alkylcarbonyl group optionally substituted with one or more substituents selected from the group consisting of OH, NH₂ and SH, in particular R₆ represents a hydrogen atom or an ethyl group, notably an ethyl group.

In a first aspect of the present invention,

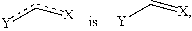

X is N, Y is N(R₂) and Z is C(H), and the compound for use according to the invention is thus of the following general formula (I.i):

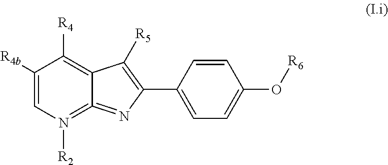

wherein R₂, R₄, R_{4b}, R₅ and R₆ are as defined in any one of the above embodiments.

In particular, R₆ represents a hydrogen atom or a (C₁-C₆)alkyl group, preferably a (C₁-C₃)alkyl group, notably a methyl or an ethyl group, advantageously R₆ represents an ethyl group.

In a second aspect of the present invention,

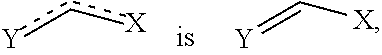

X is N(R₁), Y is N and Z is C(R₃), and the compound for use according to the invention is thus of the following general formula (I.ii.a):

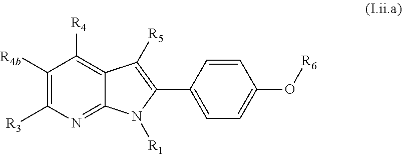

wherein R₁, R₃, R₄, R_{4b}, R₅ and R₆ are as defined in any one of the above embodiments.

In particular, R₆ represents a hydrogen atom, an ethyl or a (C₁-C₆)alkylcarbonyl group optionally substituted with one or more substituents selected from the group consisting of OH, NH₂ and SH, advantageously R₆ represents a hydrogen atom or an ethyl group, notably an ethyl group.

In a third aspect of the present invention,

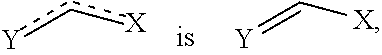

X is N(R₁), Y is N⁺(O⁻) and Z is C(R₃), and the compound for use according to the invention is thus of the following general formula (I.ii.b):

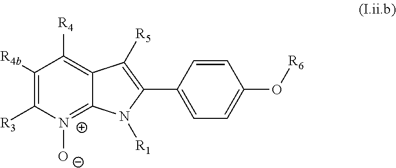

wherein R₁, R₃, R₄, R_{4b}, R₅ and R₆ are as defined in any one of the above embodiments.

In particular, R₆ represents a hydrogen atom or a (C₁-C₆)alkyl group, preferably a (C₁-C₃)alkyl group, notably a methyl or an ethyl group, advantageously R₆ represents an ethyl group.

In a fourth aspect of the present invention,

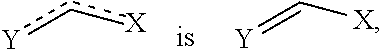

X is N(R₁), Y is CH and Z is N, and the compound for use according to the invention is thus of the following general formula (I.ii.c):

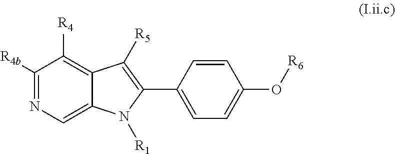

wherein R₁, R₄, R_{4b}, R₅ and R₆ are as defined in any one of the above embodiments.

In particular, R₆ represents a hydrogen atom or a (C₁-C₆)alkyl group, preferably a (C₁-C₃)alkyl group, notably a methyl or an ethyl group, advantageously R₆ represents an ethyl group.

In a fifth aspect of the present invention,

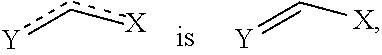

X is N(R₁) and Y and Z are CH, and the compound for use according to the invention is thus of the following general formula (I.ii.d):

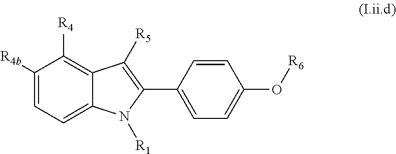

wherein R₁, R₄, R_{4b}, R₅ and R₆ are as defined in any one of the above embodiments.

In particular, R₆ represents a hydrogen atom, a (C₁-C₆)alkyl group or an aryl-(C₁-C₆)alkyl group, preferably a hydrogen atom, a (C₁-C₃)alkyl group or an aryl-(C₁-C₃)alkyl group, notably a hydrogen atom, a methyl, an ethyl or a benzyl group, advantageously R₆ represents a hydrogen atom an ethyl or a benzyl group, typically a hydrogen atom or an ethyl group.

The compound for use according to the invention may be notably selected from the group consisting of compounds 1 to 44, represented below, and the pharmaceutically acceptable salts and/or solvates thereof

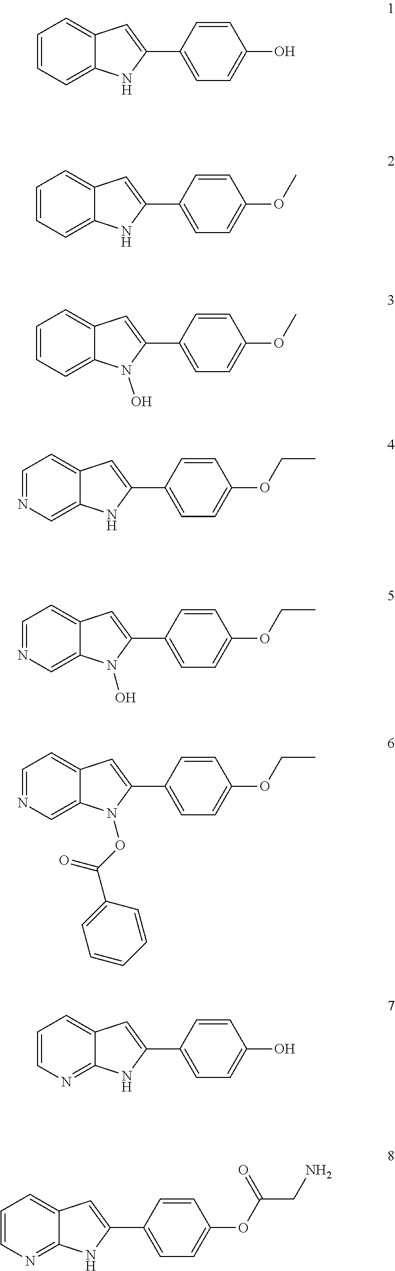

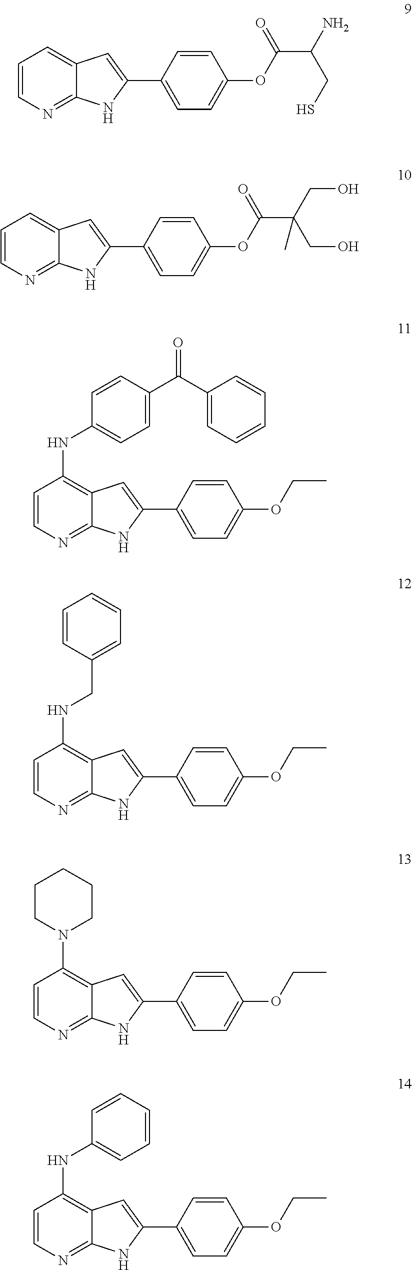 [SMILES: NC(CS)C(=O)Oc1ccc(-c2cc3cccnc3[nH]2)cc1 ; CC(CO)(CO)C(=O)Oc1ccc(-c2cc3cccnc3[nH]2)cc1 ; CCOc1ccc(-c2cc3c(Nc4ccc(C(=O)c5ccccc5)cc4)ccnc3[nH]2)cc1 ; CCOc1ccc(-c2cc3c(NCc4ccccc4)ccnc3[nH]2)cc1 ; CCOc1ccc(-c2cc3c(N4CCCCC4)ccnc3[nH]2)cc1 ; CCOc1ccc(-c2cc3c(Nc4ccccc4)ccnc3[nH]2)cc1]

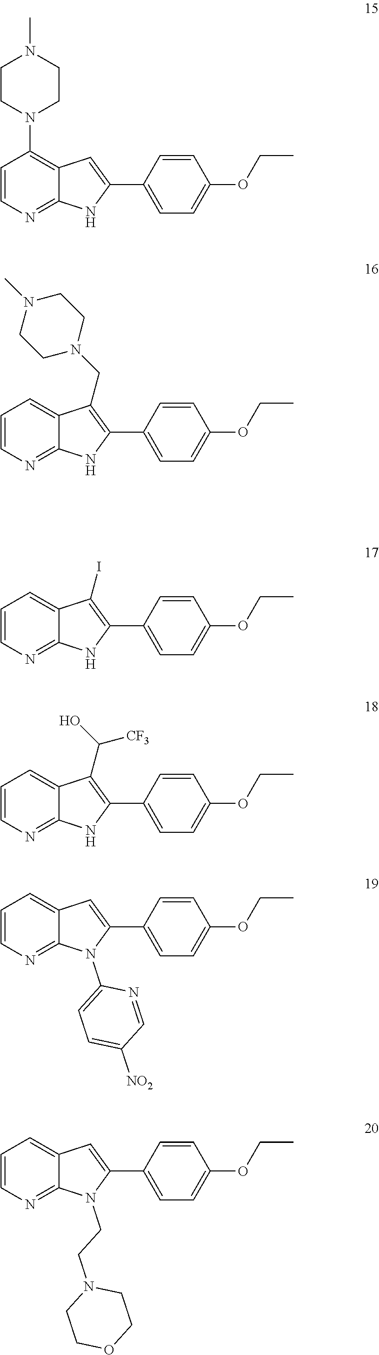 [SMILES: CCOc1ccc(-c2cc3c(N4CCN(C)CC4)ccnc3[nH]2)cc1 ; CCOc1ccc(-c2[nH]c3ncccc3c2CN2CCN(C)CC2)cc1 ; CCOc1ccc(-c2[nH]c3ncccc3c2I)cc1 ; CCOc1ccc(-c2[nH]c3ncccc3c2C(O)C(F)(F)F)cc1 ; CCOc1ccc(-c2cc3cccnc3n2-c2ccc([N+](=O)[O-])cn2)cc1 ; CCOc1ccc(-c2cc3cccnc3n2CCN2CCOCC2)cc1]

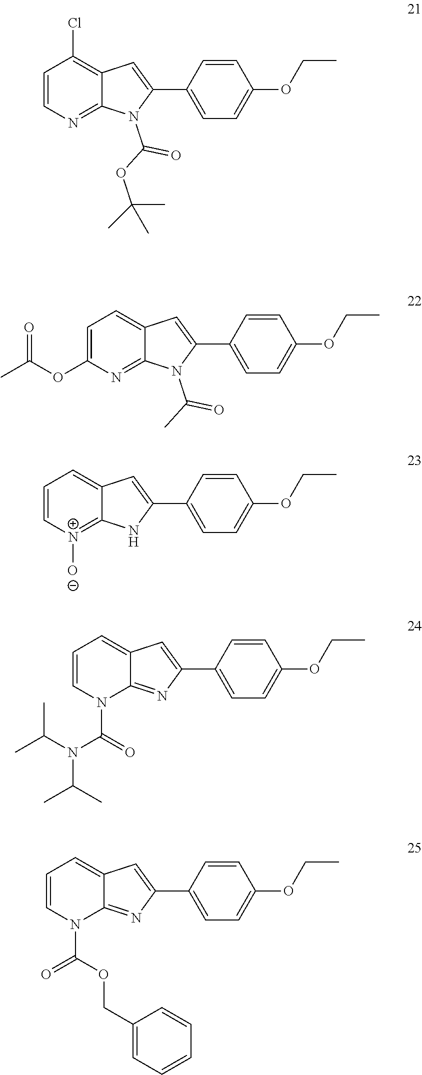

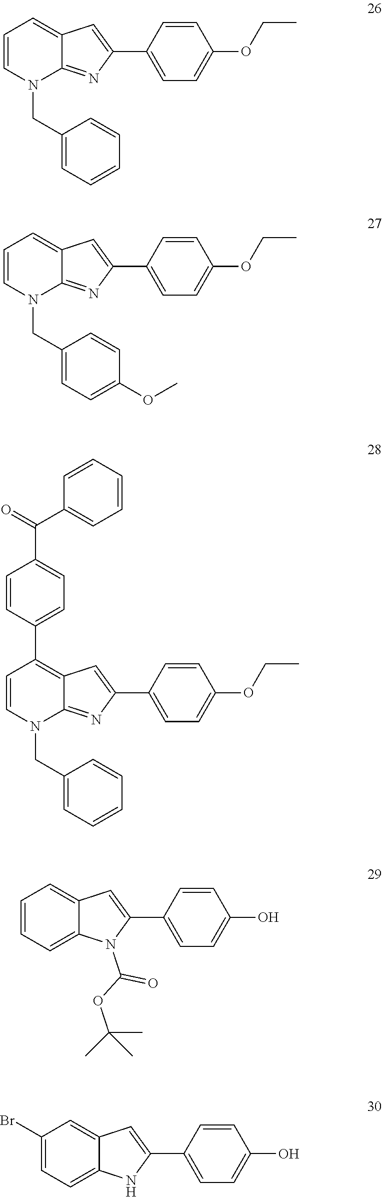 [SMILES: CCOc1ccc(-c2cc3cccn(Cc4ccccc4)c-3n2)cc1 ; CCOc1ccc(-c2cc3cccn(Cc4ccc(OC)cc4)c-3n2)cc1 ; CCOc1ccc(-c2cc3c(-c4ccc(C(=O)c5ccccc5)cc4)ccn(Cc4ccccc4)c-3n2)cc1 ; CC(C)(C)OC(=O)n1c(-c2ccc(O)cc2)cc2ccccc21 ; Oc1ccc(-c2cc3cc(Br)ccc3[nH]2)cc1]

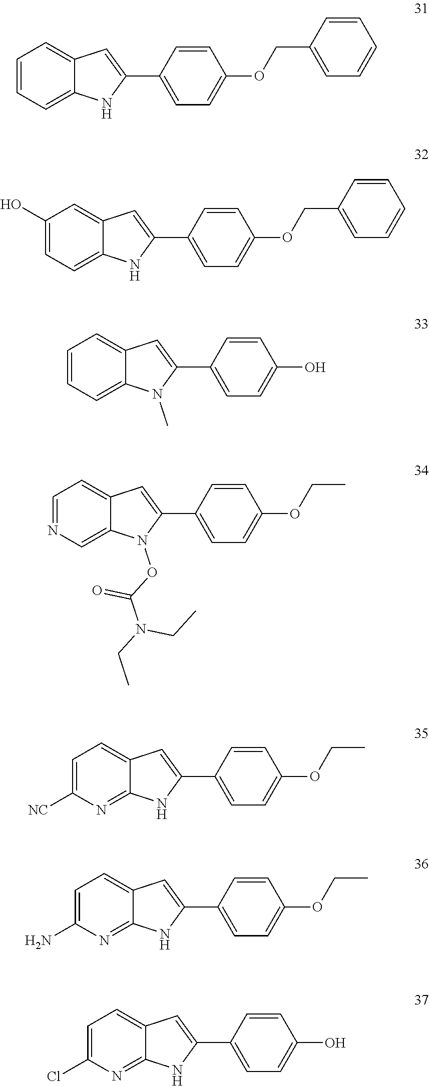

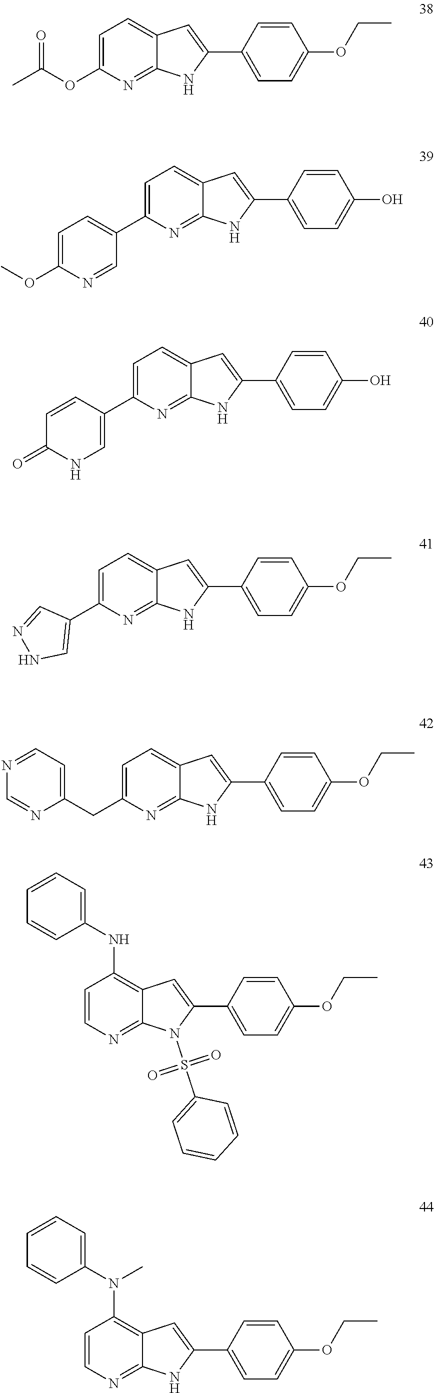

In particular, the compound for use according to the invention may be selected from the group consisting of compounds 1 to 28, and the pharmaceutically acceptable salts and/or solvates thereof.

In a particular embodiment, the compound for use according to the invention is selected from the group consisting of compounds 4-6, 8-18, 22, 28, 29, 32, 34-44, in particular 4-6, 8-18, 22, 28, 34-44, notably 4-6, 8-18, 22, 28, and the pharmaceutically acceptable salts and/or solvates thereof.

The present invention is also directed to a compound of general formula (I) as defined above, for preventing and/or treating a disorder associated with ferroptosis. The disorders associated with ferroptosis may be myocardial ischemia-reperfusion injury, notably occurring after artery ligation; cardiomyopathy, notably doxorubicin-induced cardiomyopathy; strokes, notably ischemic stroke or hemorrhagic stroke; traumatic brain injury; contusion spinal cord injury; neurodegenerative disorders, in particular chronic neurodegenerative disorders, more particularly Alzheimer's disease, Huntington's disease, Parkinson's disease, amyotrophic lateral sclerosis (Charcot's disease), Friedreich's ataxia and dementia; retinal disorders, notably Stargardt's disease or age-related macular degeneration (AMD), in particular dry AMD; chronic liver diseases, notably non-alcoholic steatohepatitis (NASH), chronic infections such as hepatitis, and alcoholic liver disease; acute liver failure, notably resulting from a drug-induced liver injury (DILI), such as acetaminophem (APAP)-induced liver injury, or from an ischemia-reperfusion injury induced by a septic or hemorrhagic shock; skin inflammatory diseases, such as psoriasis; toxic epidermal necrolysis (Lyell syndrome); acute kidney injury (AKI), such as oxalate-, folic acid (FA)- and cisplatin-induced AKI, renal ischemia-reperfusion injury and acute tubular necrosis; chronic obstructive pulmonary disease (COPD); bronchial asthma; lung injury caused by a bacterial infection, notably by Pseudomonas aeruginosa or Mycobacterium tuberculosis; pulmonary fibrosis, such as radiation induced-lung fibrosis (RILF) and paraquat-induced pulmonary damage; necrotizing enterocolitis; inflammatory bowel diseases, such as Crohn's disease; haemochromatosis; hemolytic disorders; cytokinic storm during a viral infection; radiation-induced necrosis; rheumatoid arthritis; type I diabetes; insulin resistance related to obesity; epilepsy, including mitochondrial disease-related epilepsy and intractable epilepsy; and pathologies related to stress-induced premature tissue senescence, such as atherosclerosis, hypertension and type II diabetes.

In a particular embodiment, the disorder associated with ferroptosis is selected from the group consisting of cardiomyopathy, notably doxorubicin-induced cardiomyopathy; contusion spinal cord injury; neurodegenerative disorders, in particular chronic neurodegenerative disorders, more particularly Alzheimer's disease, Huntington's disease, Parkinson's disease, amyotrophic lateral sclerosis (Charcot's disease), Friedreich's ataxia and dementia; retinal disorders, notably Stargardt's disease or age-related macular degeneration (AMD), in particular dry AMD; acute liver failure, notably resulting from a drug-induced liver injury (DILI), such as acetaminophem (APAP)-induced liver injury, or from an ischemia-reperfusion injury induced by a septic or hemorrhagic shock; skin inflammatory diseases, such as psoriasis; toxic epidermal necrolysis (Lyell syndrome); acute kidney injury (AKI), such as oxalate-, folic acid (FA)- and cisplatin-induced AKI; bronchial asthma; lung injury caused by a bacterial infection, notably by Pseudomonas aeruginosa or Mycobacterium tuberculosis; pulmonary fibrosis, such as radiation induced-lung fibrosis (RILF) and paraquat-induced pulmonary damage; necrotizing enterocolitis; haemochromatosis; hemolytic disorders; cytokinic storm during a viral infection; radiation-induced necrosis; rheumatoid arthritis; type I diabetes; insulin resistance related to obesity; epilepsy, including mitochondrial disease-related epilepsy and intractable epilepsy; and pathologies related to stress-induced premature tissue senescence, such as hypertension and type II diabetes.

Preferably, the disorder associated with ferroptosis is selected from the group consisting of myocardial ischemia-reperfusion injury, notably occurring after artery ligation; strokes, notably ischemic stroke or hemorrhagic stroke; traumatic brain injury; neurodegenerative disorders, in particular chronic neurodegenerative disorders, more particularly Alzheimer's disease, Huntington's disease, Parkinson's disease and amyotrophic lateral sclerosis (Charcot's disease); retinal disorders, notably Stargardt's disease or age-related macular degeneration (AMD), in particular dry AMD; chronic liver diseases, notably non-alcoholic steatohepatitis (NASH); acute liver failure, notably resulting from a drug-induced liver injury (DILI), such as acetaminophem (APAP)-induced liver injury, or from an ischemia-reperfusion injury induced by a septic or hemorrhagic shock; and acute kidney injury (AKI), such as folic acid (FA)-induced AKI, cisplatin-induced AKI, renal ischemia-reperfusion injury and acute tubular necrosis.

In particular, the disorder associated with ferroptosis is selected from the group consisting of neurodegenerative disorders, in particular chronic neurodegenerative disorders, more particularly Alzheimer's disease, Huntington's disease, Parkinson's disease and amyotrophic lateral sclerosis (Charcot's disease); retinal disorders, notably Stargardt's disease or age-related macular degeneration (AMD), in particular dry AMD; acute liver failure, notably resulting from a drug-induced liver injury (DILI), such as acetaminophem (APAP)-induced liver injury, or from an ischemia-reperfusion injury induced by a septic or hemorrhagic shock; and acute kidney injury (AKI), such as folic acid (FA)-induced AKI and cisplatin-induced AKI.

The present invention also relates to a method for inhibiting ferroptosis, comprising the administration to a person in need thereof of an effective dose of a compound of formula (I) as defined above. In particular, the present invention relates to a method for preventing and/or treating disorders associated with ferroptosis, as defined above, comprising the administration to a person in need thereof of an effective dose of a compound of formula (I) as defined above.

The present invention also relates to the use of a compound of formula (I) as defined above, for the manufacture of a drug for inhibiting ferroptosis. In particular, the present invention also relates to the use of a compound of formula (I) as defined above, for the manufacture of a drug for preventing and/or treating disorders associated with ferroptosis, as defined above.

The present invention also relates to the use, in particular a non-therapeutic use, of a compound of formula (I) as defined above, for the in vitro preservation and/or protection of biological materials such as cells, tissues, body fluids and organs.

In the context of the present invention, “in vitro” means outside of the organism from which the biological material derives.

As used herein, the expression “preservation and/or protection of biological materials” refers to an improved survival of said biological material, allowing its conservation over time. As is clear from the present description, this improved survival is obtained by preventing ferroptosis-induced cell death in said biological materials.

Hence, the present invention also relates to the in vitro use of a compound of formula (I) as defined above as an agent for inhibiting ferroptosis-induced cell death in a biological material.

The present invention is also directed to a method for inhibiting ferroptosis-induced cell death in a biological material, which comprises exposing said biological material to a compound of formula (I) as defined above.

In the above aspects of the present invention, the biological material is preferably a cell sample or a tissue sample.

The present invention also relates to a pharmaceutical composition comprising at least one compound of formula (I) as defined above and at least one pharmaceutically acceptable excipient, for use as a drug for inhibiting ferroptosis, particularly for preventing and/or treating disorders associated with ferroptosis, as defined above.

The term “pharmaceutically acceptable excipient” is intended to mean, in the framework of the present invention, a substance which is pharmaceutically acceptable, as defined above, formulated alongside the active ingredient(s) of the pharmaceutical composition, included for the purpose of long-term stabilization, bulking up solid formulations that contain potent active ingredients in small amounts, to confer a therapeutic improvement on the active ingredient in the final dosage form (such as facilitating drug absorption, reducing viscosity, or enhancing solubility), or to enhance the taste or the appearance of the pharmaceutical composition. The appropriate excipients can be easily and wisely selected by the skilled person, taking into account notably the dosage form and the route of administration.

The pharmaceutical compositions for use according to the invention may be formulated notably for oral administration, for topical administration or for injection, wherein said compositions are intended for mammals, including humans.

The pharmaceutical composition can be administered orally in a solid or liquid (solution or suspension) form.

A solid composition can be in the form of tablets, gelatin capsules, powders, granules and the like. When a solid composition is prepared in the form of tablets, the main active ingredient is mixed with a pharmaceutical vehicle such as gelatin, starch, lactose, magnesium stearate, talc, gum arabic and the like. The tablets may be coated with sucrose or with other suitable materials, or they may be treated in such a way that they have a prolonged or delayed activity and they continuously release a predetermined amount of active principle. In powders or granules, the active ingredient can be mixed or granulated with dispersing agents, wetting agents or suspending agents and with flavor correctors or sweeteners. In gelatin capsules, the active ingredient can be introduced into soft or hard gelatin capsules in the form of a powder or granules such as mentioned previously or in the form of a liquid composition such as mentioned below.

A liquid composition can contain the active ingredient together with a sweetener, a taste enhancer or a suitable coloring agent in a solvent such as water. The liquid composition can also be obtained by suspending or dissolving a powder or granules, as mentioned above, in a liquid such as water, juice, milk, etc. It can be for example a syrup or an elixir.

For topical administration, the pharmaceutical composition may be in any form allowing an application to the surface of the skin or mucous membranes: cream, gel, ointment, patch, etc.

For administration by injection, aqueous suspensions, isotonic saline solutions or sterile and injectable solutions which contain pharmacologically compatible dispersing agents and/or wetting agents are used.

The compounds of the invention as active ingredients may be used in doses ranging between 0.01 mg and 2,000 mg per day, given in a single dose once per day or administered in several doses throughout the day, for example twice a day in equal doses. The dose administered per day is advantageously between 5 mg and 500 mg, even more advantageously between 10 mg and 200 mg. The compounds of the invention will be typically formulated into a pharmaceutical composition as described above prior to administration to a patient. The effective dose of the active ingredient can be determined by one skilled in the art by routine tests including assessment of the effect of administration of the active ingredient on the disorders which are sought to be prevented and/or treated by said administration. For example, such tests can be implemented by analyzing both quantitative and qualitative effect of the administration of different amounts of the active ingredient on a set of markers (biological and/or clinical) characteristics of said disorder, in particular from a biological sample of a person. Besides, as is well-known to the skilled person, the suitable dose and the associated dosing regimen for treating a given disease in a given patient will depend on several other factors, such as the stage of the disease as well as the physical and medical condition of the patient.

The present invention also relates to a compound of formula (I) or a pharmaceutical composition as defined above for use as a therapeutically active ingredient in a combination or in an add-on therapeutic regimen in a person in need thereof, in particular for inhibiting ferroptosis, more particularly for preventing and/or treating disorder associated with ferroptosis.

In some embodiments, the compound of formula (I) of the present invention is administered simultaneously, separately or sequentially to a person in need thereof with a second active ingredient.

The compound of formula (I) and/or the pharmaceutical composition as defined above may be provided in a combination product, comprising additional products, in particular a second active ingredient, particularly intended for simultaneously, separately or sequentially administration.

The second active ingredient is typically relevant for the disorder to be prevented and/or treated.

The present invention also relates to a kit comprising a pharmaceutical composition as defined above and a delivery device (a device allowing administration of said composition), in particular suitable for parenteral, enteral administration or local administration. Examples of delivery devices include but are not limited to autoinjectors, in particular multichamber syringes, transdermal patchs, pre-filled syringe or a needle free device.

The present invention is also directed to a compound selected from the group consisting of

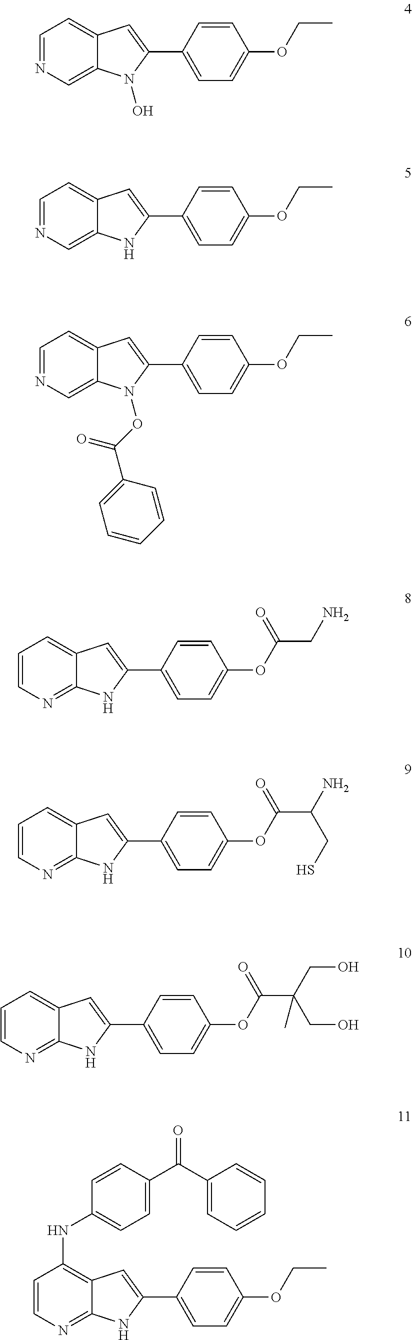

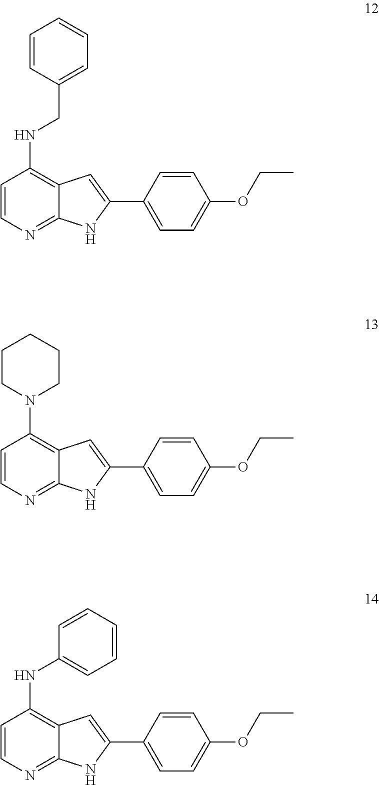 [SMILES: CCOc1ccc(-c2cc3c(NCc4ccccc4)ccnc3[nH]2)cc1 ; CCOc1ccc(-c2cc3c(N4CCCCC4)ccnc3[nH]2)cc1 ; CCOc1ccc(-c2cc3c(Nc4ccccc4)ccnc3[nH]2)cc1]

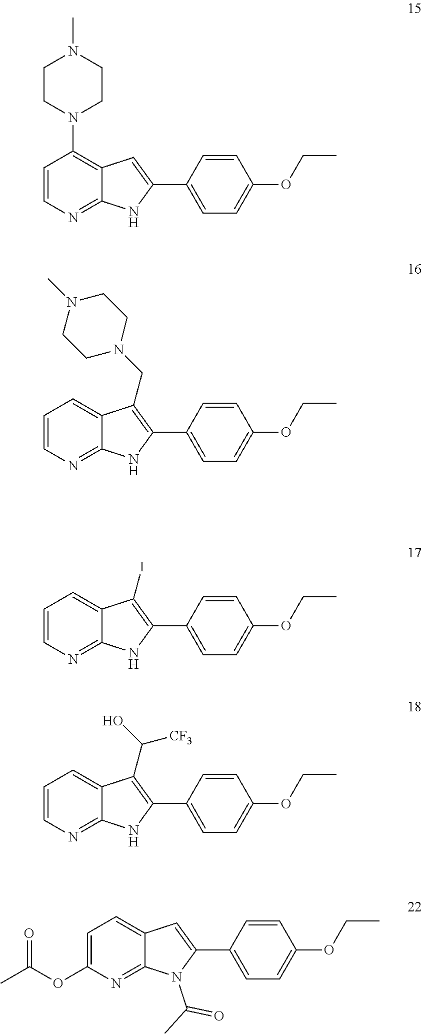

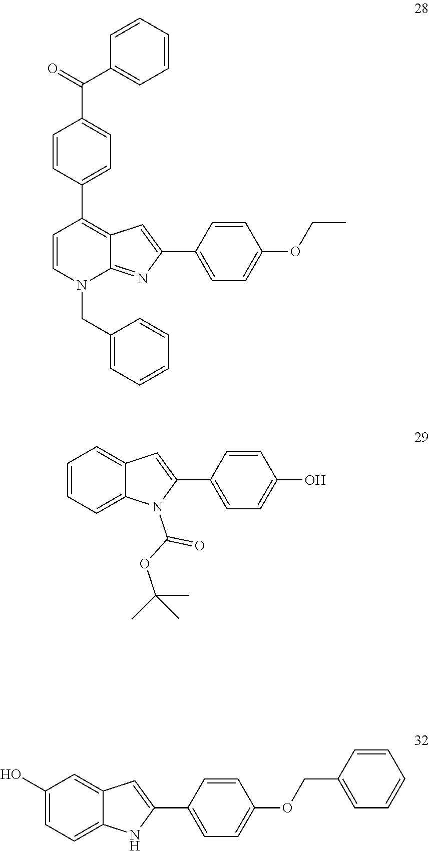 [SMILES: CCOc1ccc(-c2cc3c(-c4ccc(C(=O)c5ccccc5)cc4)ccn(Cc4ccccc4)c-3n2)cc1 ; CC(C)(C)OC(=O)n1c(-c2ccc(O)cc2)cc2ccccc21 ; Oc1ccc2[nH]c(-c3ccc(OCc4ccccc4)cc3)cc2c1]

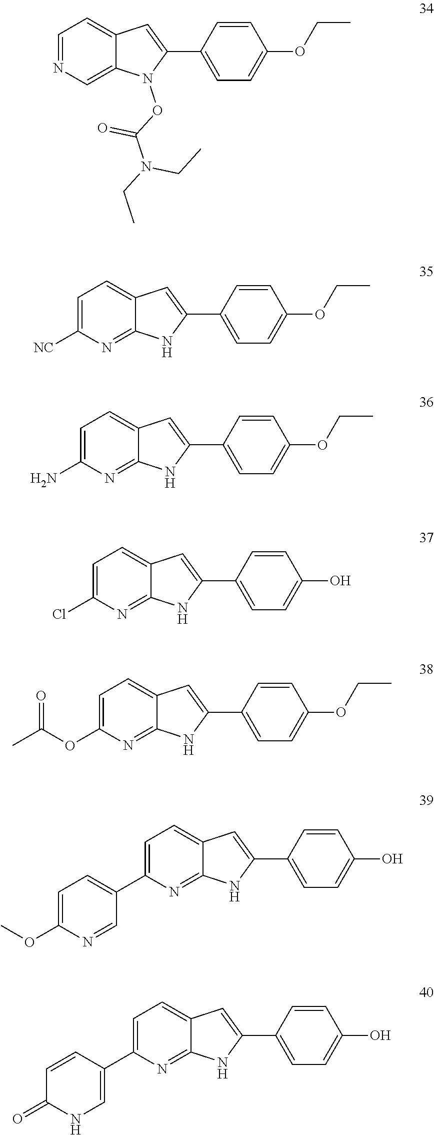

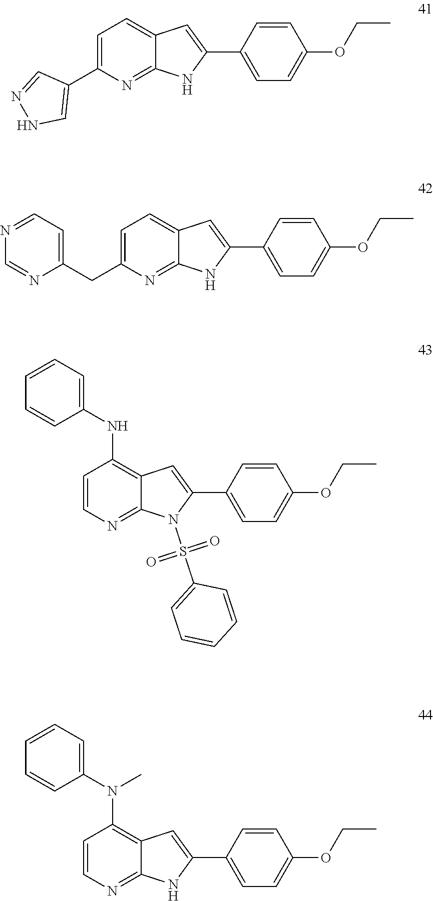 [SMILES: CCOc1ccc(-c2cc3ccc(-c4cn[nH]c4)nc3[nH]2)cc1 ; CCOc1ccc(-c2cc3ccc(Cc4ccncn4)nc3[nH]2)cc1 ; CCOc1ccc(-c2cc3c(Nc4ccccc4)ccnc3n2S(=O)(=O)c2ccccc2)cc1 ; CCOc1ccc(-c2cc3c(N(C)c4ccccc4)ccnc3[nH]2)cc1]

and the pharmaceutically acceptable salts and/or solvates thereof.

In particular, the compound according to the invention is selected from the group consisting of compounds 4-6, 8-18, 22, 28, 34-44, notably 4-6, 8-18, 22, 28, and the pharmaceutically acceptable salts and/or solvates thereof.

The present invention relates also to a compound selected from the group consisting of compounds 4-6, 8-18, 22, 28, 29, 32, 34-44, notably 4-6, 8-18, 22, 28, 34-44, in particular 4-6, 8-18, 22, 28, and the pharmaceutically acceptable salts and/or solvates thereof, for use as a drug.

### BRIEF SUMMARY OF THE FIGURES

FIG. 1 represents the dose-dependent protection from ferroptosis induced by erastin (Era), glutamate (Glu) or RSL3 ((1S,3R)-RAS-selective lethal) of mouse hippocampal neuronal cell line HT22 by compound 6.

FIG. 2A represents the dose-dependent protection from ferroptosis, induced by erastin (Era), of human neuroblastoma cell line SH-SY5Y by compound 6.

FIG. 2B represents the dose-dependent protection from ferroptosis, induced by erastin (Era) or RSL3 ((1S,3R)-RAS-selective lethal), of human neuroblastoma cell line SH-SY5Y by compound 25.

FIG. 3 represents the ischemia-reperfusion model used in example 11.2.

FIGS. 4-16 represent the viability of human renal glomerular endothelial cells (hRGEC) treated with increasing concentrations of compounds 1, 27, 5, 26, 25, 14, 11, 6, 12, 15, 13, 22 and 20 after hypoxic cold storage (4° C., 24 h) and reoxygenation step (37° C., 6 h).

FIGS. 17-18 represent the viability of human renal glomerular endothelial cells (hRGEC) treated with increasing concentrations of compounds 24 and 7 after hypoxic cold storage (4° C., 24 h) and reoxygenation step (37° C., 4 h (grey) and 6 h (black)).

FIGS. 19-24 represent the viability of rat kidney cells (NRK-52E) treated with increasing concentrations of 24 compounds of the invention after a 24 h treatment with 200 μm of cisplastin.

FIG. 25 represents the dose-dependent protection of a human retinal pigment epithelial cells (ARPE-19 cell line) from the NaIO₃-induced retinal cell death by compounds 7, 1, 8, 9, 27, 25, 22 and 24.

FIGS. 26-28 represent the dose-dependent protection from ferroptosis, induced by erastin (Era), of human neuroblastoma cell line SH-SY5Y by compounds 16, 17 and 18.

FIG. 29 shows the cell survival of RPE cells after treatment with compound 1 (SBL-02) before induction of phototoxicity compared to crocetine (CRO), positive control (CONT-A2E) and negative control (CONT+A2E).

FIG. 30 shows photopic ERG measured at an intensity of 30 cd·s/m2 in mice treated in intravitreal (IVT) with compound 1 (SBL2) or its vehicle. I: injected eye, NI: non-injected eye, NINI: untreated and non-injected mouse. N=4 VEH mice, n=5 SBL2 mice and n=5 NINI mice.

FIGS. 31 and 32 show scotopic ERG A (FIG. 31) and B (FIG. 32) measured at five increasing light intensities in mice that received IVT compound 1 (SBL2 I) or its vehicle (VEH I). Uninjected eyes of the same mice serve as controls (SBL2 NI and VEH NI). Uninjected and unlit mice (NINI) serve as positive controls. N=4 VEH mice, n=5 SBL2 mice and n=5 NINI mice.

FIG. 33 shows the quantification of the thickness of the retinas (expressed as distance from the optic nerve) of mice with or without intravitreal treatment with compound 1 (SBL2).

FIGS. 34 and 35 show scotopic ERG A (FIG. 34) and B (FIG. 35) measured at five increasing light intensities in mice treated with compound 7 (SBL1), its placebo (VEH) and non-injected non-illuminated (NINI). N=3 mice per injected group and n=5 for NINI.

FIG. 36 shows the quantification of the thickness of the retinas of mice with or without intraperitoneal treatment with compound 7 (SBL2).

FIGS. 37-39 show the effect of compounds 7 (SBL-01), 1 (SBL-02), 14 (SBL-571), 22 (SBL-962) and 3 (SBL-1495) on dopaminergic neurons (TH staining) survival after 6-OHDA injury.

FIGS. 40 and 41 show the creatinine and urea plasma level in the anesthetised rat model of ischaemic acute renal failure induced by transient bilateral renal artery occlusion in the rat, with or without treatment with compound 7 (sibiriline).

### EXAMPLES

The following abbreviations, commonly used in this field of art, are used in the following examples:
A2E: N-retinylidene-N-retinylethanolamine
BDNF: Brain Derived Neurotrophic Factor
BLD: Blue light damage
BOC: tert-butyloxycarbonyl
BSA: Bovine Serum Albumin
nBuLi n-Butyllithium
BUN blood urea nitrogen
CD cyclodextrin
DDQ: 2,3-dichloro-5,6-dicyano-p-benzoquinone
DIMAP: 4-Dimethylaminopyridine
DMEM: Dulbecco's Modified Eagle Medium
DMF: Dimethylformamide
DMSO: Dimethylsulfoxyde
EC₅₀: Half maximal effective concentration
EDCI: 1-Ethyl-3-(3-dimethylaminopropyl)carbodiimide
ENT: ear, nose, throat
ERG: electroretinogram
Et: Ethyl (CH₂CH₃)
EtOAc: Ethyl acetate
FBS: fetal bovine serum
FCS: fetal calf serum
HRGEC: Human Renal Glomerular Endothelial Cells
hRPE-1: human Retinal Pigment Epithelial cell line
IP: intraperitoneal
IVT: intravitreal
LDA: Lithium diisopropylamide
Me: Methyl (CH₃)
Bis-MPA: 2,2-Bis(hydroxymethyl)propionic acid
MsCl Methanesulfonyl chloride
MTS: 3-[4,5-dimethylthiazol-2-yl]-5-[3-carboxymethoxy-phenyl]-2-[4-sulfophenyl]-2H-tetrazolium
n: number of replicates in an experiment
NMR: Nuclear Magnetic Resonance
OCT: optical coherence tomography
6-OHDA: 6-hydroxydopamine
PBS: Phosphate buffered saline
PE: petroleum ether
PMBCl: 4-Methoxybenzyl chloride
iPrOH: isopropanol
nPrOH: n-propanol.
RBF: round-bottom flask
ROS: reactive oxygen species
RPE: retinal pigment epithelium
r.t.: Room temperature
SD: Standard Deviation
TH: Tyrosine Hydroxylase
THF: tetrahydrofuran
TLC: Thin Layer Chromatography
XTT 2,3-Bis-(2-methoxy 4-nitro-5-sulfophenyl)-2H-tetrazolium5-carboxanilide salt

I. Synthesis of the Compounds According to the Invention

General procedures A and B detailed below have been followed for preparing several compounds according to the invention.

### General Procedure A: Buchwald-Hartwig C—N Coupling of 4-chloro-2-(4-ethoxyphenyl)-1H-pyrrolo[2,3-b]pyridine

4-chloro-2-(4-ethoxyphenyl)-1H-pyrrolo[2,3-b]pyridine (50 mg, 0.18 mmol) was charged into a vial with Pd₂dba₃ (17 mg, 0.1 eq, 0.018 mmol), XPhos (17 mg, 0.2 eq, 0.036 mmol), tBuONa (45 mg, 3 eq, 0.54 mmol) and the corresponding amine (1.3 eq, 0.23 mmol). The vial was placed under argon and dry dioxane was added before stirring 16 h at 100° C. After cooling the reaction was diluted with EtOAc, filtered on a pad of Celite® and concentrated. The crude product was purified by flash chromatography to afford the desired compound.

### General Procedure B: Direct Amination of 4-chloro-2-(4-ethoxyphenyl)-1H-pyrrolo[2,3-b]pyridine

4-chloro-2-(4-ethoxyphenyl)-1H-pyrrolo[2,3-b]pyridine (30 mg, 0.11 mmol) was charged into a microwave 0.5 ml vial with phenol (21 mg, 2 eq, 0.22 mmol) and the corresponding amine (2 eq, 0.22 mmol). The vial was placed under argon and heated to 150° C. (neat) for 30 min in a Biotage Initiator microwave reactor. After cooling the reaction was diluted with EtOAc, washed with brine, dried over MgSO₄ and concentrated. The crude product was purified by flash chromatography to afford the desired compound.

Both procedures relies on the use of 4-chloro-2-(4-ethoxyphenyl)-1H-pyrrolo[2,3-b]pyridine as starting material. Another key compound has been used for preparing several compounds according to the invention, namely 2-(4-ethoxyphenyl)-1H-pyrrolo[2,3-b]pyridine.

### Synthesis of 4-chloro-2-(4-ethoxyphenyl)-1H-pyrrolo[2,3-b]pyridine

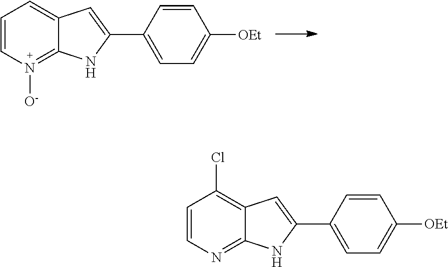

2-(4-ethoxyphenyl)-1H-pyrrolo[2,3-b]pyridine-7-oxide (compound 23) (1 g, 4.1 mmol) was dissolved in anhydrous DMF (10 ml) under argon and MsCl was added dropwise (487 μl, 6.15 mmol, 1.5 eq). The reaction was heated to 80° C. and stirred for 6 h before cooling with an ice bath to yield a precipitate. Water (40 ml) was added and the yellow solid was filtered, washed with more water and dried under vacuum (696 mg, 65%). ¹H-NMR (300 MHz, DMSO-d₆): 1.35 (t, J=7.0 Hz, 3H), 4.09 (q, J=7.0 Hz, 2H), 6.85 (d, J=2.1 Hz, 1H), 7.02 (d, J=8.8 Hz, 2H), 7.17 (d, J=5.2 Hz, 1H), 7.92 (d, J=8.8 Hz, 2H), 8.12 (d, J=5.2 Hz, 1H), 12.39 (bs, 1H).

### Synthesis of 2-(4-ethoxyphenyl)-1H-pyrrolo[2,3-b]pyridine

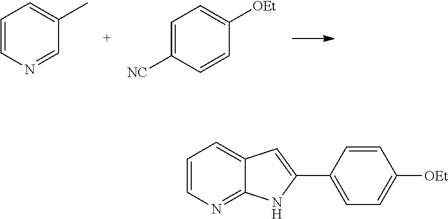

LDA was freshly prepared by adding dropwise a nBuLi solution in hexanes (54 ml, 2.5 M, 135 mmol) to a diisopropylamine (19 ml, 135 mmol) solution in anhydrous THF (150 ml) at −5° C. and stirring for 20 min. Then a solution of 3-picoline (7 g, 75 mmol, 1 eq) in anhydrous THF (100 ml) was added dropwise at 0° C. and the orange mixture was stirred for 20 min before dropwise addition of a solution of 4-ethoxybenzonitrile (11.1 g, 75 mmol, 1 eq) in anhydrous THF (100 ml). After 1 h at 0° C., more LDA solution in THF (150 ml) was added dropwise (135 mmol, prepared from 54 ml nBuLi and 19 ml diisopropylamine) and the reaction was slowly warmed to r.t during 1 h before being heated to reflux in a water bath for 2 h. After cooling, the yellow solution was quenched carefully with saturated NH₄Cl (100 ml) and water (250 ml) was added. The precipitate was filtered, washed with diethyl ether and water, dried under vacuum to afford alight-yellow solid (11 g, 61%).

¹H-NMR (300 MHz, DMSO-d₆): 1.35 (t, J=7.1 Hz, 3H), 4.07 (q, J=7.1 Hz, 2H), 6.78 (d, J=2.2 Hz, 1H), 7.00-7.05 (m, 3H), 7.85-7.89 (m, 3H), 8.15 (dd, J=4.7, 1.6 Hz, 1H), 12.0 (bs, 1H).

Other starting materials in the syntheses detailed below are either commercially available, or can be easily prepared according to methods well-known of the one skilled in the art.

### Compound 1: 4-(1H-indol-2-yl)phenol

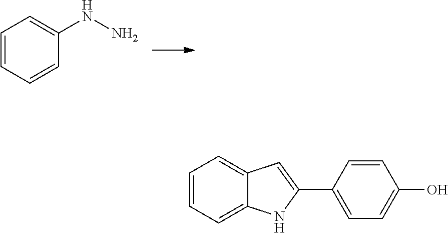 [SMILES: NNc1ccccc1 ; Oc1ccc(-c2cc3ccccc3[nH]2)cc1]

4-(1H-indol-2-yl)phenol was obtained by Fisher indole synthesis, using phenylhydrazine and 4-hydroxyacetophenone as starting materials, as described in Eur. J. Med. Chem., 2014, 74, 477-490. The desired compound was obtained as a brown solid (68%).

¹H NMR (300 MHz; DMSO-d₆): 11.30 (br s, 1H), 9.62 (br s, 1H); 7.69 (d, 2H, J=7.2 Hz); 7.45 (d, 1H, J=7.7 Hz); 7.34 (d, 1H, J=7.7 Hz); 7.06-6.93 (m, 2H); 6.82 (d, 2H, J=7.2 Hz); 6.68 (s, 1H).

### Compound 2: 2-(4-methoxyphenyl)-1H-indole

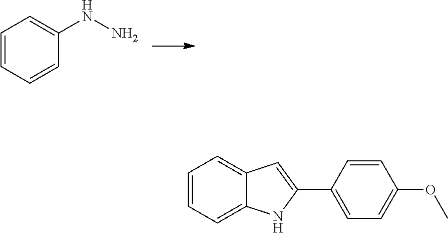 [SMILES: NNc1ccccc1 ; COc1ccc(-c2cc3ccccc3[nH]2)cc1]

2-(4-methoxyphenyl)-1H-indole was obtained by Fisher indole synthesis, using phenylhydrazine and 4-methoxyacetophenone as starting materials, as described in Bioorg. Med. Chem. Lett., 2013, 23, 2671-2674. The desired compound was obtained as a light brown solid (56%).

¹H NMR (300 MHz, CDCl₃): 8.50 (br s, 1H), 7.61-7.58 (m, 3H), 7.39 (d, J=7.2 Hz, 1H), 7.17 (dt, J=7.2 Hz, 1.3 Hz, 1H), 7.10 (dt, J=7.2 Hz, 1.3 Hz, 1H), 6.98 (d, J=8.7 Hz, 2H), 6.71 (s, 1H), 3.87 (s, 3H).

### Compound 3: 2-(4-methoxyphenyl)-1H-indol-1-ol

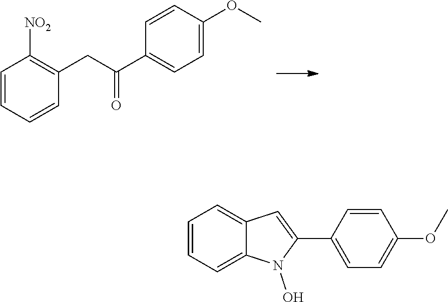 [SMILES: COc1ccc(C(=O)Cc2ccccc2[N+](=O)[O-])cc1 ; COc1ccc(-c2cc3ccccc3n2O)cc1]

1-(4-methoxyphenyl)-2-(2-nitrophenyl)ethan-1-one (500 mg, 1.84 mmol) was dissolved in EtOAc:EtOH (9:1, 10 ml) and tin chloride (1.47 g, 4.2 eq, 7.74 mmol) was added in one portion and the mixture was stirred at r.t. for 48 h. Brine was added and the aqueous phase was extracted with EtOAc (2×30 ml), washed multiple times with water, dried over MgSO₄ and concentrated to a red oil. Purification was performed by flash chromatography (pure EtOAc then 80:20 to 50:50 EtOAc:MeOH gradient). The solid obtained was suspended in water, sonicated, filtered and washed thoroughly with water. Drying under vacuum afforded a light beige solid (313 mg, 71%).

¹H NMR (300 MHz, DMSO-d₆) 11.11 (s, 1H), 7.81 (d, J=9.0 Hz, 2H), 7.50 (d, J=7.8 Hz, 1H), 7.39 (d, J=7.8 Hz, 1H), 7.18-7.11 (m, 1H), 7.12-6.94 (m, 3H), 6.52 (d, J=0.8 Hz, 1H), 3.81 (s, 3H).

### Compound 4: 2-(4-ethoxyphenyl)-1H-pyrrolo[2,3-c]pyridine

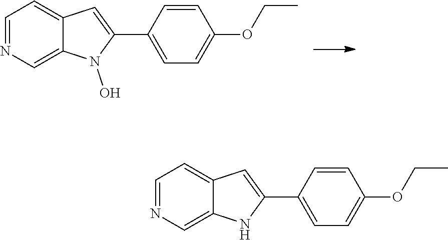 [SMILES: CCOc1ccc(-c2cc3ccncc3n2O)cc1 ; CCOc1ccc(-c2cc3ccncc3[nH]2)cc1]

2-(4-ethoxyphenyl)-1Hpyrrolo[2,3-c]pyridin-1-ol (50 mg, 0.2 mmol) was suspended in a mixture of acetic acid and ethanol (1:2, 9 ml) and iron powder was added (220 mg, 20 eq, 3.9 mmol). The suspension was heated to reflux for 1 h and cooled to r.t. Saturated NaHCO₃ was carefully added and the reaction mixture was extracted with EtOAc (3×20 ml), washed with brine, dried over MgSO₄, filtered and concentrated. The crude product was purified by flash chromatography (100:0 to 90:10 CH₂Cl₂:MeOH) to afford a light yellow solid (31 mg, 63%).

¹H NMR (300 MHz, Methanol-d₄): 8.71 (s, 1H), 8.06 (d, J=6.0 Hz, 1H), 7.92-7.80 (m, 2H), 7.75 (d, J=6.0 Hz, 1H), 7.13-7.03 (m, 2H), 7.00 (s, 1H), 4.11 (q, J=7.0 Hz, 2H), 1.42 (t, J=7.0 Hz, 3H).

### Compound 5: 2-(4-ethoxyphenyl)-1Hpyrrolo[2,3-c]pyridin-1-ol

### Synthesis of 4-(4-ethoxystyryl)-3-nitropyridine

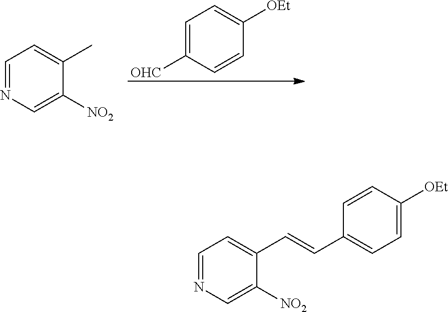

4-methyl-3-nitropyridine (7 g, 51 mmol) and 4-ethoxybenzaldehyde (8 g, 1.05 eq, 54 mmol) were placed into a 120 ml pressure tube and dissolved in EtOH (60 ml). A catalytic amount of piperidine was added and the reaction was stirred at 110° C. until consumption of the starting material (approx. 72 to 96 h). The mixture was cooled to r.t., concentrated to about 25 ml volume and placed in the freezer, resulting in the formation of yellow crystals. Filtration and washing with minimum cold EtOH yielded a solid that was purified by recrystallization from a mixture of hexane:EtOAc (9:1). The crystals were filtered, washed with hexane and dried under vacuum to afford a bright yellow solid (11.3 g, 82%).

¹H NMR (300 MHz, CDCl₃) δ 9.14 (s, 1H), 8.69 (d, J=5.4 Hz, 1H), 7.66 (d, J=5.4 Hz, 1H), 7.55-7.44 (m, 3H), 7.31 (d, J=16.1 Hz, 1H), 6.92 (d, J=6.8 Hz, 2H), 4.08 (q, J=7.0 Hz, 2H), 1.44 (t, J=7.0 Hz, 3H).

### Synthesis of N-(4-(4-ethoxystyryl)pyridin-3-yl)hydroxylamine

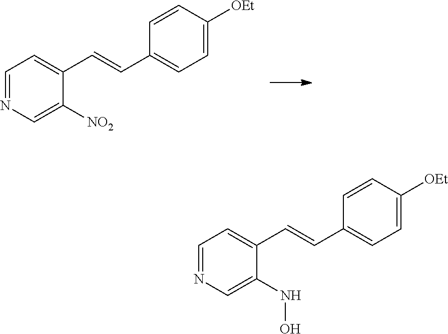 [SMILES: CCOc1ccc(/C=C/c2ccncc2[N+](=O)[O-])cc1 ; CCOc1ccc(/C=C/c2ccncc2NO)cc1]

4-(4-ethoxystyryl)-3-nitropyridine (11 g, 41 mmol) was dissolved in THF:MeOH mixture (1:1, 600 ml) and cooled to 0° C. Sodium acetate (55.8 g, 10 eq, 410 mmol) was added, followed by tin chloride (46.3 g, 5 eq, 205 mmol) in portions. The reaction was stirred 5 h while returning to r.t., producing a thick orange slurry. TLC analysis showed no starting material and the mixture was poured into ice cold saturated NaHCO₃ (1.5 L). The suspension was extracted with EtOAc:iPrOH (8:2, 4×600 ml), the organic extracts were washed with saturated NaHCO₃, brine and dried over MgSO₄. Removal of the solvent afforded a light yellow solid (8.9 g, 85%).

¹H NMR (300 MHz, DMSO-d₆) δ 8.64 (s, 1H), 8.54 (d, J=1.8 Hz, 1H), 8.38 (s, 1H), 8.00 (d, J=5.0 Hz, 1H), 7.58 (d, J=8.8 Hz, 2H), 7.44 (d, J=5.0 Hz, 1H), 7.27 (d, J=16.2 Hz, 1H), 7.15 (d, J=16.2 Hz, 1H), 6.96 (d, J=8.8 Hz, 2H), 4.06 (q, J=7.0 Hz, 2H), 1.34 (t, J=7.0 Hz, 3H).

### Synthesis of 2-(4-ethoxyphenyl)-1H-pyrrolo[2,3-c]pyridin-1-ol

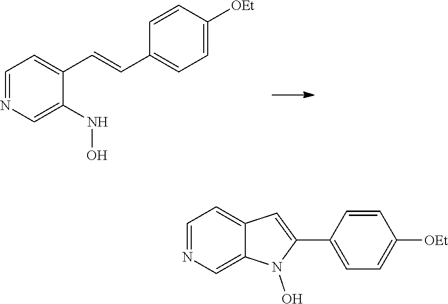 [SMILES: CCOc1ccc(/C=C/c2ccncc2NO)cc1 ; CCOc1ccc(-c2cc3ccncc3n2O)cc1]

N-(4-(4-ethoxystyryl)pyridin-3-yl)hydroxylamine (9 g, 35 mmol) was suspended in a mixture of MeCN:H₂O:AcOH (10:2:1, 390 ml) and cooled to −5-0° C. DDQ (7.94 g, 1 eq, 35 mmol) was added in small portions, to obtain and dark red solution that was stirred for 30 min before TLC analysis, that showed no remaining starting material. The crude was quenched by addition of 1N HCl (100 ml) and diluted with Et₂O (300 ml). The organic phase was extracted with 1N HCl and the combined aqueous layer was washed with Et₂O:PE (1:1) until no DDQ byproducts were extracted (3×500 ml). The aqueous layer was then transferred to an Erlenmeyer and made basic with saturated NaHCO₃ under vigorous stirring. The precipitate was filtered, washed with water, EtOAc and finally Et₂O before being dried under vacuum to afford a brown solid (7.11 g, 80%).

¹H NMR (300 MHz, DMSO-d₆) δ 11.79 (s, 1H), 8.73 (s, 1H), 8.10 (d, J=5.4 Hz, 1H), 7.88 (d, J=8.8 Hz, 2H), 7.46 (dd, J=5.4, 1.0 Hz, 1H), 7.06 (d, J=8.8 Hz, 2H), 6.59 (s, 1H), 4.10 (q, J=7.0 Hz, 2H), 3.16 (d, J=4.9 Hz, 1H), 1.36 (t, J=7.0 Hz, 4H).

### Compound 6: 2-(4-ethoxyphenyl)-1H-pyrrolo[2,3-c]pyridin-1-yl benzoate

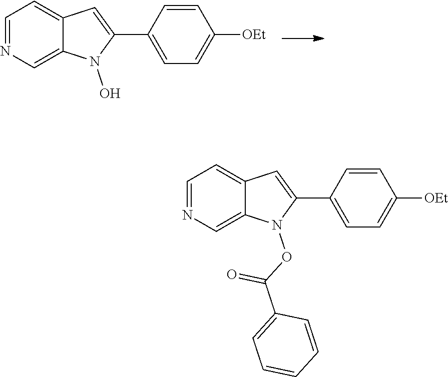 [SMILES: CCOc1ccc(-c2cc3ccncc3n2O)cc1 ; CCOc1ccc(-c2cc3ccncc3n2OC(=O)c2ccccc2)cc1]

(2-(4-ethoxyphenyl)-1H-pyrrolo[2,3-c]pyridin-1-ol (compound 5) (1.95 g, 7.7 mmol) was placed under argon and dissolved in anhydrous CH₂Cl₂ (50 ml). Anhydrous pyridine (1.81 g, 1.8 ml, 23 mmol) was added, the mixture was cooled to about −15° C. and benzoyl chloride (1.33 ml, 1.6 g, 12 mmol) was added dropwise. The mixture was stirred for 1 h while returning to 0° C. and TLC analysis showed total conversion. The reaction was quenched at 0° C. with saturated NaHCO₃, extracted with CH₂Cl₂ (2×50 ml), dried over MgSO₄ and concentrated to a brown oil. The product was purified by flash chromatography (Et₃N-neutralized silica gel, CH₂Cl₂:MeOH gradient) to afford 1.2 g of product, contaminated by an impurity. Further purification was performed by dissolution in minimum Et₂₀, addition of PE and sonication with shaking until formation of a precipitate. The suspension was filtered and dried under vacuum to obtain a tan solid (912 mg, 32%). ¹H NMR (300 MHz, CDCl₃) δ 8.67 (s, 1H), 8.35 (s, 1H), 8.12 (d, J=7.2 Hz, 2H), 7.73-7.65 (m, 1H), 7.62 (d, J=8.8 Hz, 2H), 7.52 (t, J=7.8 Hz, 3H), 6.91 (d, J=8.8 Hz, 2H), 6.62 (s, 1H), 4.02 (q, J=7.0 Hz, 2H), 1.39 (t, J=7.0 Hz, 3H).

### Compound 7: 4-(1H-pyrrolo[2,3-b]pyridin-2-yl)phenol

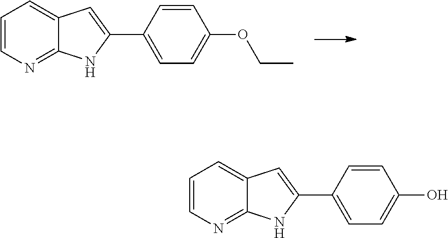 [SMILES: CCOc1ccc(-c2cc3cccnc3[nH]2)cc1 ; Oc1ccc(-c2cc3cccnc3[nH]2)cc1]

2-(4-methoxyphenyl)-1H-pyrrolo[2,3-b]pyridine (1.06 g, 47.2 mmol) was placed in a 250 ml 3 neck RBF equiped with an argon inlet and a thermometer, and suspended in anhydrous CH₂Cl₂ (50 ml), cooled to −78° C. Boron tribromide (1M solution in CH₂Cl₂, 141.7 mmol, 3 eq) was added dropwise and the dark brown mixture was stirred 6 h while slowly warming to r.t. The reaction was carefully quenched with saturated NaHCO₃ solution (20 ml) and cooled to 0° C. before addition of 2M NaOH until the precipitated solid was mostly dissolved (50 ml). The solution was stirred for 10 min before being loaded in a separating funnel and washed with dichloromethane. The aqueous phase was then neutralized with dropwise addition of 6M HCl under stirring. Filtration gave a brown solid, washed with saturated NaHCO₃ solution and water to give 828 mg a crude product after hivac drying. Purification was performed by recrystallization in nPrOH to which a small amount of hexane is added (8 ml and 3 ml) before heating to reflux. The solution was cooled to r.t. and placed at −20° C. overnight. Filtration afforded a light brown solid (535 mg, 54%).

¹H-NMR (300 MHz, CDCl₃): 6.70 (d, J=2.0 Hz, 1H), 6.85 (d, J=8.7 Hz, 2H), 7.02 (dd, J=7.8, 4.7 Hz, 1H), 7.76 (d, J=8.7 Hz, 2H), 7.85 (dd, J=7.8, 1.3 Hz, 1H), 8.14 (dd, J=4.7, 1.3 Hz, 1H), 9.71 (bs, 1H), 11.93 (bs, 1H).

### Compound 8: 4-(1H-pyrrolo[2,3-b]pyridin-2-yl)phenyl glycinate

### 4-(1H-pyrrolo[2,3-b]pyridin-2-yl)phenyl (tert-butoxycarbonyl) glycinate

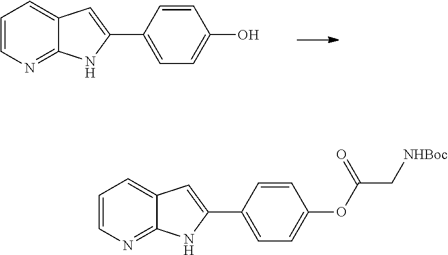 [SMILES: Oc1ccc(-c2cc3cccnc3[nH]2)cc1 ; CC(C)(C)OC(=O)NCC(=O)Oc1ccc(-c2cc3cccnc3[nH]2)cc1]

4-(1H-pyrrolo[2,3-b]pyridin-2-yl)phenol (compound 7) (180 mg, 0.87 mmol) was suspended in CH₂Cl₂ (6 ml) with DMAP (138 mg, 1.3 eq, 1.13 mmol), Boc-Gly-OH (182 mg, 1.2 eq, 1.04 mmol) and EDCI-HCl (210 mg, 1.3 eq, 1.13 mmol) was added. The mixture was stirred at r.t. for 16 h before addition of water and EtOAc (15 ml each). The crude mixture was extracted (2×15 ml), washed with brine, dried over MgSO₄ and concentrated to a solid. Purification was performed by flash chromatography (80:20 to 0:100 PE:EtOAc) to afford a white solid (120 mg, 37%).

¹H NMR (300 MHz, DMSO-d₆) δ 12.15 (s, 1H), 8.22 (dd, J=4.7, 1.6 Hz, 1H), 8.06-7.88 (m, 3H), 7.44 (t, J=5.3 Hz, 1H), 7.23 (d, J=8.8 Hz, 2H), 7.07 (dd, J=7.8, 4.7 Hz, 1H), 6.92 (d, J=2.1 Hz, 1H), 4.00 (d, J=6.0 Hz, 2H), 1.42 (s, 9H).

### Synthesis of 4-(1H-pyrrolo[2,3-b]pyridin-2-yl)phenyl glycinate hydrochloride

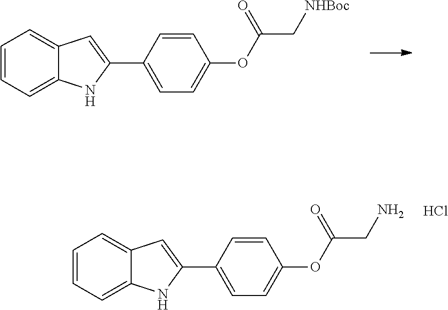 [SMILES: CC(C)(C)OC(=O)NCC(=O)Oc1ccc(-c2cc3ccccc3[nH]2)cc1 ; NCC(=O)Oc1ccc(-c2cc3ccccc3[nH]2)cc1 ; Cl]

4-(1H-pyrrolo[2,3-b]pyridin-2-yl)phenyl (tert-butoxycarbonyl)glycinate (110 mg, 0.29 mmol) was dissolved in anhydrous dioxane (5 ml) and dry HCl (4M in dioxane, 2.9 ml, 40 eq, 11.6 mmol) was added dropwise. The suspension was stirred at r.t. for 16 h and filtered. The resulting paste was washed with EtOAc, CH₂Cl₂ and dried under vacuum to afford a white solid (100 mg, quantitative) containing residual dioxane.

¹H NMR (300 MHz, DMSO-d₆) 12.77 (s, 1H), 8.64 (br s, 3H), 8.30 (dd, J=5.0, 1.5 Hz, 1H), 8.18 (d, J=7.9 Hz, 1H), 8.08 (d, J=8.8 Hz, 2H), 7.33 (d, J=8.8 Hz, 2H), 7.24 (dd, J=7.9, 5.0 Hz, 1H), 7.07 (d, J=1.8 Hz, 1H), 4.13 (d, J=5.6 Hz, 2H).

### Compound 9: 4-(1H-pyrrolo[2,3-b]pyridin-2-yl)phenyl L-cysteinate

### 4-(1H-pyrrolo[2,3-b]pyridin-2-yl)phenyl (tert-butoxycarbonyl)-L-cysteinate

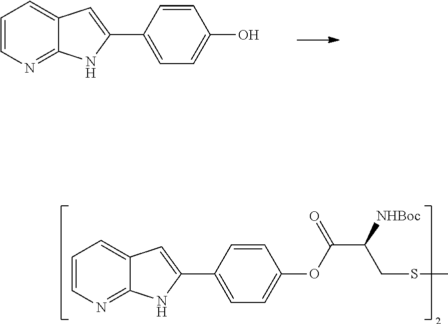

4-(1H-pyrrolo[2,3-b]pyridin-2-yl)phenol (compound 7) (50 mg, 2 eq, 0.24 mmol) was suspended in CH₂Cl₂ (5 ml) with DMAP (35 mg, 2.4 eq, 0.29 mmol), (Boc-Cys-OH)₂ (52 mg, 1 eq, 0.12 mmol) and EDCI-HCl (59 mg, 2.4 eq, 0.29 mmol) was added. The mixture was stirred at r.t. for 16 h before concentration of the solvent and addition of water (20 ml). The resulting suspension was sonicated, filtered and washed with water, EtOAc, CH₂Cl₂ and MeOH. Drying under vacuum afforded a light beige solid (52 mg, 53%), isolated as the disulfide.

¹H NMR (400 MHz, DMSO-d₆) δ 12.15 (s, 2H), 8.21 (d, J=4.5 Hz, 2H), 7.98 (d, J=8.5 Hz, 4H), 7.92 (d, J=8.0 Hz, 2H), 7.78-7.68 (m, 2H), 7.22 (d, J=8.5 Hz, 4H), 7.06 (dd, J=8.0, 4.5 Hz, 2H), 6.90 (d, J=1.5 Hz, 2H), 4.55 (br s, 2H), 3.37 (t, J=11.6 Hz, 2H, obscured by water peak), 3.17 (t, J=11.6 Hz, 2H), 1.43 (s, 18H).

### Synthesis of 4-(1H-pyrrolo[2,3-b]pyridin-2-yl)phenyl L-cysteinate dihydrochloride

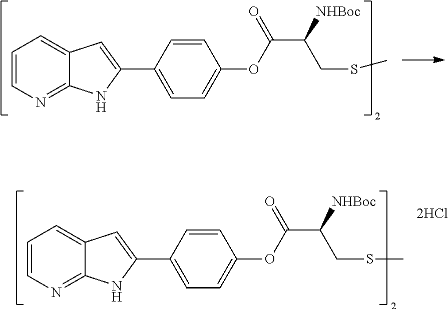

4-(1H-pyrrolo[2,3-b]pyridin-2-yl)phenyl (tert-butoxycarbonyl)-L-cysteinate (60 mg, 0.07 mmol) was dissolved in anhydrous dioxane (5 ml) and dry HCl (4M in dioxane, 720 μl, 40 eq, 2.9 mmol) was added dropwise. The suspension was stirred at r.t. for 16 h and filtered. The resulting paste was washed with CH₂Cl₂, EtOAc, acetone, MeOH and dried under vacuum to afford a beige solid, corresponding to compound 9 in its oxidized form (45 mg, 90%).

¹H NMR (300 MHz, DMSO-d₆) δ 12.53 (s, 2H), 9.11 (s, 6H), 8.27 (dd, J=4.9, 1.5 Hz, 2H), 8.06 (d, J=8.8 Hz, 6H), 7.37 (d, J=8.8 Hz, 4H), 7.17 (dd, J=7.8, 4.9 Hz, 2H), 6.99 (d, J=1.8 Hz, 2H), 3.65-3.58 (m, 4H).

### Compound 10: 4-(1H-pyrrolo[2,3-b]pyridin-2-yl)phenyl

### 3-hydroxy-2-(hydroxymethyl)-2-methylpropanoate

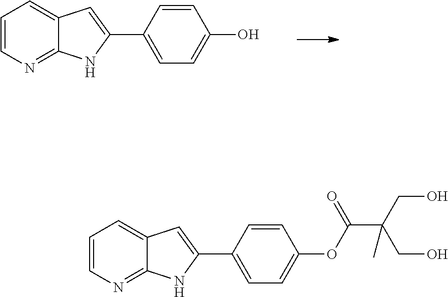 [SMILES: Oc1ccc(-c2cc3cccnc3[nH]2)cc1 ; CC(CO)(CO)C(=O)Oc1ccc(-c2cc3cccnc3[nH]2)cc1]

4-(1H-pyrrolo[2,3-b]pyridin-2-yl)phenol (compound 7) (20 mg, 0.09 mmol) was suspended in CH₂Cl₂ (5 ml) with DMAP (14 mg, 1.2 eq, 0.11 mmol), 2,2,5-trimethyl-1,3-dioxane-5-carboxylic acid (17 mg, 1 eq, 0.09 mmol) and EDCI-HCl (22 mg, 1.2 eq, 0.11 mmol) was added. The mixture was stirred at r.t. for 16 h before concentration of the solvent, addition of water (10 ml) and EtOAc (10 ml). The mixture was extracted with EtOAc, washed with brine, dried over MgSO₄ and concentrated to afford the protected bis-MPA ester as a white solid (40 mg) which was used without further purification. The crude product was dissolved in THF (5 ml) with aqueous HCl (200 μl, excess) and stirred at r.t. for 1 h. The reaction was quenched with saturated NaHCO₃, extracted with EtOAc (2×20 ml) and the organic phase was washed with brine, dried over MgSO₄ and concentrated. Purification was performed by flash chromatography (100:0 to 90:10 CH₂Cl₂:MeOH gradient) to obtain a white solid (15 mg, 48%).

¹H NMR (300 MHz, DMSO-d₆) δ 12.14 (s, 1H), 8.21 (dd, J=4.7, 1.6 Hz, 1H), 8.07-7.86 (m, 3H), 7.18 (d, J=8.7 Hz, 2H), 7.07 (dd, J=7.8, 4.7 Hz, 1H), 6.92 (d, J=2.1 Hz, 1H), 4.95 (t, J=5.5 Hz, 2H), 3.70 (dd, J=10.5, 5.5 Hz, 2H), 3.55 (dd, J=10.5, 5.5 Hz, 2H), 1.23 (s, 3H).

### Compound 11: (4-((2-(4-ethoxyphenyl)-1H-pyrrolo[2,3-b]pyridin-4-yl)amino)phenyl)(phenyl)methanone

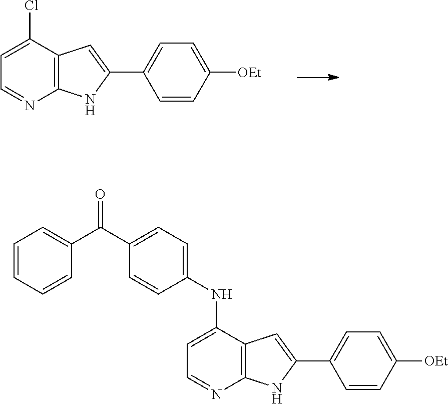 [SMILES: CCOc1ccc(-c2cc3c(Cl)ccnc3[nH]2)cc1 ; CCOc1ccc(-c2cc3c(Nc4ccc(C(=O)c5ccccc5)cc4)ccnc3[nH]2)cc1]

Following the general procedure A with 4-aminobenzophenone (37 mg, 46%).

¹H-NMR (300 MHz, DMSO-d₆): 1.35 (t, J=7.0 Hz, 3H), 4.08 (q, J=7.0 Hz, 2H), 6.89 (d, J=2.1 Hz, 1H), 6.97 (d, J=5.5 Hz, 1H), 7.02 (d, J=8.9 Hz, 2H), 7.40 (d, J=8.8 Hz, 2H), 7.51-7.69 (m, 3H), 7.71-7.82 (m, 6H), 8.02 (d, J=5.5 Hz, 1H), 9.23 (bs, 1H), 11.95 (bs, 1H).

### Compound 12: N-benzyl-2-(4-ethoxyphenyl)-1H-pyrrolo[2,3-b]pyridin-4-amine

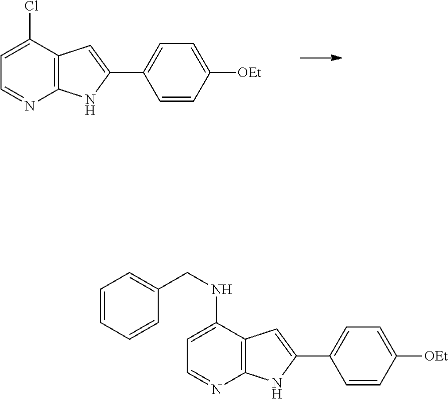 [SMILES: CCOc1ccc(-c2cc3c(Cl)ccnc3[nH]2)cc1 ; CCOc1ccc(-c2cc3c(NCc4ccccc4)ccnc3[nH]2)cc1]

Following the general procedure A with benzylamine (32 mg, 51%).

¹H-NMR (300 MHz, DMSO-d₆): 1.35 (t, J=7.0 Hz, 3H), 4.07 (q, J=7.0 Hz, 2H), 4.49 (d, J=6.0 Hz, 2H), 6.05 (d, J=5.6 Hz, 1H), 6.90 (d, J=1.9 Hz, 1H), 6.99 (d, J=8.9 Hz, 2H), 7.19-7.25 (m, 2H), 7.30-7.42 (m, 4H), 7.65-7.78 (m, 3H), 11.58 (bs, 1H).

### Compound 13: 2-(4-ethoxyphenyl)-4-(piperidin-1-yl)-1H-pyrrolo[2,3-b]pyridine

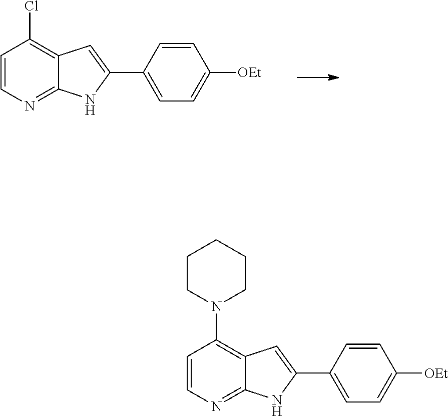 [SMILES: CCOc1ccc(-c2cc3c(Cl)ccnc3[nH]2)cc1 ; CCOc1ccc(-c2cc3c(N4CCCCC4)ccnc3[nH]2)cc1]

Following the general procedure B with piperidine (11 mg, 31%).

¹H-NMR (300 MHz, DMSO-d₆): 1.34 (t, J=7.0 Hz, 3H), 1.60-1.72 (m, 6H), 3.36-3.41 (m, 4H), 4.07 (q, J=7.0 Hz, 2H), 6.39 (d, J=5.5 Hz, 1H), 6.78 (s, 1H), 6.98 (d, J=8.8 Hz, 2H), 7.84 (d, J=8.8 Hz, 2H), 7.90 (d, J=5.5 Hz, 1H), 11.77 (bs, 1H).

### Compound 14: 2-(4-ethoxyphenyl)-N-phenyl-1H-pyrrolo[2,3-b]pyridin-4-amine

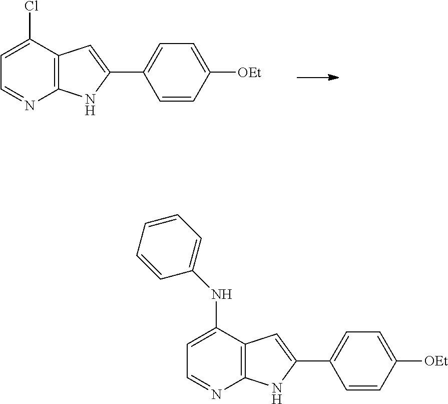 [SMILES: CCOc1ccc(-c2cc3c(Cl)ccnc3[nH]2)cc1 ; CCOc1ccc(-c2cc3c(Nc4ccccc4)ccnc3[nH]2)cc1]

Following the general procedure A with aniline (18 mg, 30%).

¹H-NMR (300 MHz, MeOD-d₄): 1.43 (t, J=7.0 Hz, 3H), 4.10 (q, J=7.0 Hz, 2H), 6.75 (d, J=5.9 Hz, 1H), 6.82 (s, 1H), 7.00 (d, J=8.8 Hz, 2H), 7.13 (t, J=6.8 Hz, 1H), 7.32-7.45 (m, 4H), 7.72 (d, J=8.9 Hz, 2H), 7.85 (d, J=5.9 Hz, 1H).

### Compound 15: 2-(4-ethoxyphenyl)-4-(4-methylpiperazin-1-yl)-1H-pyrrolo[2,3-b]pyridine

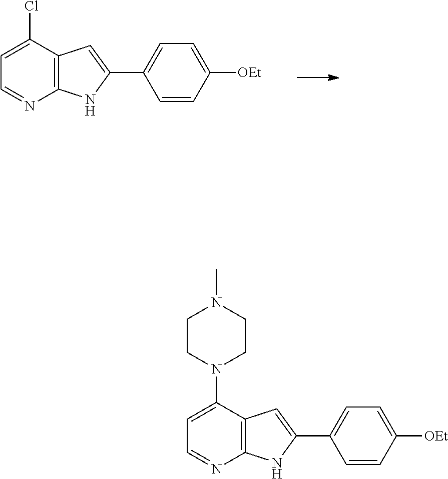 [SMILES: CCOc1ccc(-c2cc3c(Cl)ccnc3[nH]2)cc1 ; CCOc1ccc(-c2cc3c(N4CCN(C)CC4)ccnc3[nH]2)cc1]

Following the general procedure B with N-methylpiperazine (5 mg, 13%).

¹H-NMR (300 MHz, MeOD-d₄): 1.41 (t, J=6.9 Hz, 3H), 2.41 (s, 3H), 2.69-2.73 (d, J=4.5 Hz, 4H), 3.51-3.56 (d, J=4.5 Hz, 4H), 4.08 (q, J=6.9 Hz, 2H), 6.50 (s, 1H), 6.72 (s, 1H), 6.98 (d, J=8.7 Hz, 2H), 7.73 (d, J=8.7 Hz, 2H), 7.92 (s, 1H).

### Compound 16: 2-(4-ethoxyphenyl)-3-((4-methylpiperazin-1-yl)methyl)-1H-pyrrolo[2,3-b]pyridine

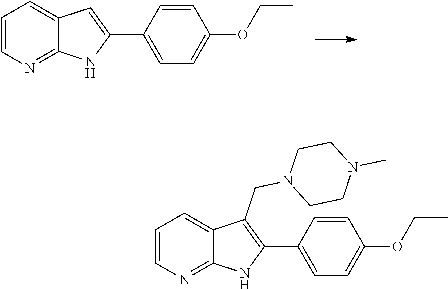 [SMILES: CCOc1ccc(-c2cc3cccnc3[nH]2)cc1 ; CCOc1ccc(-c2[nH]c3ncccc3c2CN2CCN(C)CC2)cc1]

2-(4-ethoxyphenyl)-1H-pyrrolo[2,3-b]pyridine (100 mg, 0.42 mmol) was added to a solution of N-methylpiperazine (235 μL, 5 eq, 2.1 mmol) and formaldehyde (37% aqueous, 170 μL, 5 eq, 2.1 mmol) in acetic acid (5 ml). The solution was heated to 70° C. and stirred for 18 h. The mixture was concentrated under vacuum and quenched with saturated NaHCO₃. The residue was extracted with CH₂Cl₂ (3×30 ml), washed with brine, dried over MgSO₄ and concentrated to a yellow paste. Purification was performed by flash chromatography (98:2 to 92:8 CH₂Cl₂:MeOH) to afford a light beige solid (85 mg, 58%).

¹H NMR (300 MHz, CDCl₃) 11.39 (s, 1H), 8.18 (d, J=3.8 Hz, 1H), 8.05 (d, J=7.9 Hz, 1H), 7.76 (d, J=8.7 Hz, 2H), 7.16-7.01 (m, 3H), 4.14 (q, J=7.0 Hz, 2H), 3.81 (br s, 2H), 2.79 (br s, 8H), 2.52 (s, 3H), 1.49 (t, J=7.0 Hz, 3H).

### Compound 17: 2-(4-ethoxyphenyl)-3-iodo-1H-pyrrolo[2,3-b]pyridine

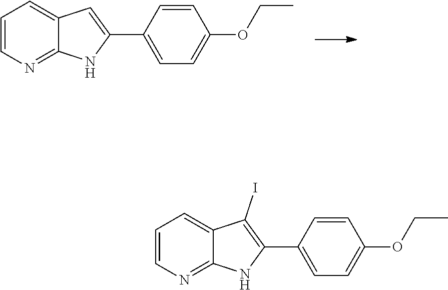 [SMILES: CCOc1ccc(-c2cc3cccnc3[nH]2)cc1 ; CCOc1ccc(-c2[nH]c3ncccc3c2I)cc1]

2-(4-ethoxyphenyl)-1H-pyrrolo[2,3-b]pyridine (400 mg, 1.67 mmol) was suspended in CH₂Cl₂ (10 ml) at 0° C. and N-iodosuccinimide (415 mg, 1.1 eq, 1.84 mmol) was added portionwise. The mixture was stirred while returning to r.t. for 2 h and concentrated under vacuum. The residue was suspended in water (20 ml), filtered, washed with CH₂Cl₂, Et₂₀, water and dried under vacuum to afford a brown solid (220 mg, 36%).

¹H NMR (300 MHz, DMSO-d₆) 8.25 (d, J=4.7 Hz, 1H), 7.79 (d, J=8.0 Hz, 2H), 7.68 (d, J=7.8 Hz, 1H), 7.20-6.99 (m, 3H), 4.12 (q, J=7.0 Hz, 2H), 1.37 (t, J=7.0 Hz, 3H).

### Compound 18: 1-(2-(4-ethoxyphenyl)-1H-pyrrolo[2,3-b]pyridin-3-yl)-2,2,2-trifluoroethan-1-ol

### 1-(2-(4-ethoxyphenyl)-1H-pyrrolo[2,3-b]pyridin-3-yl)-2,2,2-trifluoroethan-1-one

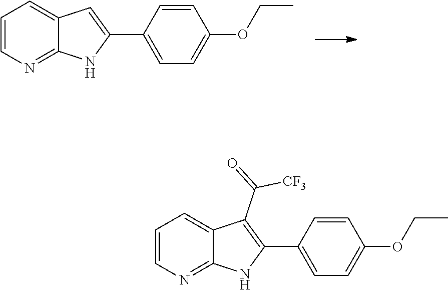 [SMILES: CCOc1ccc(-c2cc3cccnc3[nH]2)cc1 ; CCOc1ccc(-c2[nH]c3ncccc3c2C(=O)C(F)(F)F)cc1]

2-(4-ethoxyphenyl)-1H-pyrrolo[2,3-b]pyridine (400 mg, 1.67 mmol) was dissolved in anhydrous DMF (10 ml), trifluoroacetic anhydride (700 μL, 3 eq, 5.03 mmol) was added dropwise at r.t. and the mixture was heated to 70° C. for 48 h. TLC analysis showed residual starting material and more trifluoroacetic anhydride (3 eq) was added, with heating for 24 h. The reaction was cooled to r.t., quenched with dropwise addition of water (10 ml) and concentrated under vacuum. The residue was suspended in water and extracted with EtOAc (3×20 ml), washed with brine, dried over MgSO₄ and concentrated to a brown solid. Purification was performed by flash chromatography (90:10 to 60:40 EtOAc:PE) to afford a light brown solid (480 mg, 86%).

¹H NMR (300 MHz, CDCl₃) 10.85 (br s, 1H), 8.79 (d, J=8.1 Hz, 1H), 8.28 (d, J=5.3 Hz, 1H), 7.66 (d, J=8.7 Hz, 2H), 7.57-7.53 (m, 1H), 7.03 (d, J=8.7 Hz, 2H), 4.13 (q, J=7.0 Hz, 2H), 1.46 (t, J=7.0 Hz, 3H).

### 1-(2-(4-ethoxyphenyl)-1H-pyrrolo[2,3-b]pyridin-3-yl)-2,2,2-trifluoroethan-1-ol

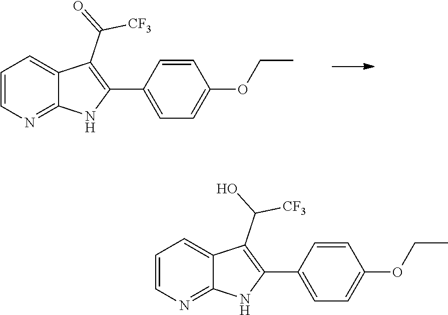 [SMILES: CCOc1ccc(-c2[nH]c3ncccc3c2C(=O)C(F)(F)F)cc1 ; CCOc1ccc(-c2[nH]c3ncccc3c2C(O)C(F)(F)F)cc1]

1-(2-(4-ethoxyphenyl)-1H-pyrrolo[2,3-b]pyridin-3-yl)-2,2,2-trifluoroethan-1-one (50 mg, 0.15 mmol) was suspended in methanol (5 ml), cooled to 0° C. and sodium borohydride (6 mg, 1 eq, 0.15 mmol) was added. The reaction was stirred for 3 h while returning to r.t., quenched with water and extracted with EtOAc (2×10 ml). The organic phase was washed with brine, dried over MgSO₄ and concentrated to a white solid (47 mg, 95%).

¹H NMR (300 MHz, CDCl₃) 8.40 (d, J=7.8 Hz, 3H), 8.14-8.05 (m, 1H), 7.50 (d, J=8.7 Hz, 2H), 7.23-7.16 (m, 1H), 7.01 (d, J=8.7 Hz, 2H), 5.41 (q, J=7.2 Hz, 1H), 4.11 (q, J=7.0 Hz, 2H), 1.47 (t, J=7.0 Hz, 3H).

### Compound 19: 2-(4-ethoxyphenyl)-1-(5-nitropyridin-2-yl)-1H-pyrrolo[2,3-b]pyridine

### Synthesis of 2-chloro-3-((4-ethoxyphenyl)ethynyl)pyridine

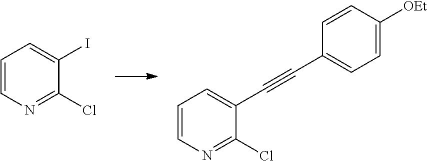 [SMILES: Clc1ncccc1I ; CCOc1ccc(C#Cc2cccnc2Cl)cc1]

2-chloro-3-iodopyridine (500 mg, 2.09 mmol) was placed under argon and dissolved in anhydrous THF (10 ml). Et₃N (1.44 ml, 5 eq, 10 mmol) and 1-ethoxy-4-ethynylbenzene (365 mg, 1.2 eq, 2.5 mmol) were added, followed by CuI (10 mg, 0.025 eq, 0.05 mmol) and PdCl₂(PPh₃)₂ (37 mg, 0.025 eq, 0.05 mmol). The dark brown mixture was stirred at r.t. for 3 h, before water (20 ml) and CH₂Cl₂ (20 ml) were added. The aqueous layer was extracted with CH₂Cl₂ and the organic extracts were washed with water and brine, dried over MgSO₄ and concentrated. The crude product was purified by flash chromatography (PE:EtOAc 85:15) to afford the desired compound (440 mg, 82%).

¹H-NMR (300 MHz, CDCl₃): 1.43 (t, J=7.0 Hz, 3H), 4.06 (q, J=7.0 Hz, 2H), 6.87 (d, J=8.8 Hz, 2H), 7.22 (dd, J=7.7, 4.8 Hz, 1H), 7.49 (d, J=8.8 Hz, 2H), 7.82 (dd, J=7.7, 1.9 Hz, 1H), 8.31 (dd, J=4.8, 1.9 Hz, 1H).

### Synthesis of 2-(4-ethoxyphenyl)-1-(5-nitropyridin-2-yl)-1H-pyrrolo[2,3-b]pyridine

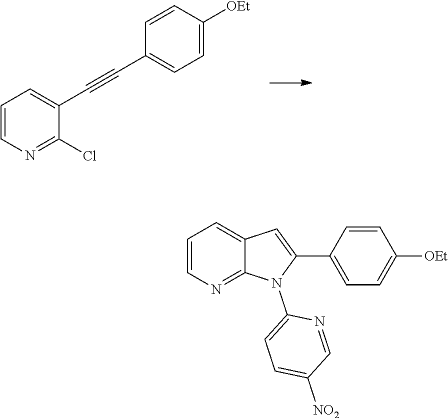 [SMILES: CCOc1ccc(C#Cc2cccnc2Cl)cc1 ; CCOc1ccc(-c2cc3cccnc3n2-c2ccc([N+](=O)[O-])cn2)cc1]

2-chloro-3-((4-ethoxyphenyl)ethynyl)pyridine (50 mg, 0.19 mmol) was charged into a vial with Pd₂dba₃ (18 mg, 0.1 eq, 0.019 mmol), XantPhos (22 mg, 0.2 eq, 0.036 mmol), Cs₂CO₃ (189 mg, 3 eq, 0.58 mmol) and 2-amino-5-nitropyridine (34 mg, 1.3 eq, 0.25 mmol). The vial was placed under argon and anhydrous dioxane was added before stirring 16 h at 100° C. After cooling the reaction was diluted with EtOAc, filtered on a pad of Celite® and concentrated. The crude product was purified by flash chromatography (90:10 EtOAc:PE) to afford a yellow solid (17 mg, 24%).

¹H-NMR (300 MHz, CDCl₃): 1.45 (t, J=7.0 Hz, 3H), 4.06 (q, J=7.0 Hz, 2H), 6.74 (s, 1H), 6.81-6.90 (m, 2H), 7.15-7.28 (m, 3H), 7.95-8.02 (m, 2H), 8.34 (d, J=4.1 Hz, 1H), 8.63 (dd, J=8.9, 2.8 Hz, 1H), 9.27 (d, J=2.4 Hz, 1H).

### Compound 20: 4-(2-(2-(4-ethoxyphenyl)-1H-pyrrolo[2,3-b]pyridin-1-yl)ethyl)morpholine

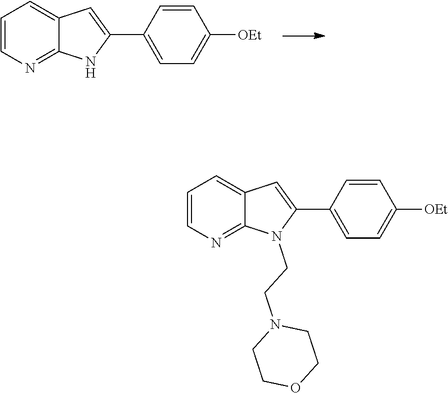 [SMILES: CCOc1ccc(-c2cc3cccnc3[nH]2)cc1 ; CCOc1ccc(-c2cc3cccnc3n2CCN2CCOCC2)cc1]

2-(4-ethoxyphenyl)-1H-pyrrolo[2,3-b]pyridine (50 mg, 0.21 mmol) was dissolved in anhydrous DMF (2 ml) under argon, treated with K₂CO₃ (145 mg, 5 eq, 1.05 mmol) and stirred at r.t. for 15 min before adding 4-(2-Chloroethyl)morpholine hydrochloride (58 mg, 1.5 eq, 0.32 mmol). The mixture was stirred 18 h at 70° C., concentrated under vacuum and the residue was purified by flash chromatography (98:2 CH₂Cl₂:MeOH) to afford the N1-regioisomer as a yellow oil (19 mg, 26%). ¹H-NMR (300 MHz, CDCl₃) for N1-substituted compound: 1.46 (t, J=7.0 Hz, 3H), 2.23-2.39 (m, 4H), 2.63 (t, J=6.9 Hz, 2H), 3.46-3.62 (m, 4H), 4.10 (q, J=7.0 Hz, 2H), 4.46 (t, J=6.9 Hz, 2H), 6.41 (s, 1H), 6.95-7.03 (m, 2H), 7.06 (dd, J=7.8, 4.8 Hz, 1H), 7.44-7.51 (m, 2H), 7.87 (dd, J=7.8, 1.6 Hz, 1H), 8.30 (dd, J=4.8, 1.6 Hz, 1H).

### Compound 21: tert-butyl 4-chloro-2-(4-ethoxyphenyl)-1H-pyrrolo[2,3-b]pyridine-1-carboxylate

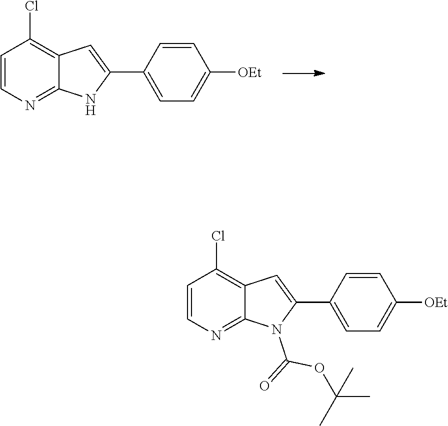 [SMILES: CCOc1ccc(-c2cc3c(Cl)ccnc3[nH]2)cc1 ; CCOc1ccc(-c2cc3c(Cl)ccnc3n2C(=O)OC(C)(C)C)cc1]

4-chloro-2-(4-ethoxyphenyl)-1H-pyrrolo[2,3-b]pyridine (50 mg, 0.18 mmol) was suspended in anhydrous CH₂Cl₂ (5 ml) under argon, DMAP (5 mg) was added, followed by Et₃N (28 μL, 1.1 eq, 0.20 mmol) and Boc₂O (43 mg, 1.1 eq, 0.20 mmol) and the mixture was stirred at r.t. for 4 h before adding water. The organic layer was washed with brine, dried over MgSO₄, filtered and concentrated. The crude product was purified by flash chromatography (90:10 PE:EtOAc) to afford a white solid (45 mg, 71%).

¹H-NMR (300 MHz, CDCl₃): 1.32 (s, 9H), 1.45 (t, J=7.0 Hz, 3H), 4.09 (q, J=7.0 Hz, 2H), 6.56 (s, 1H), 6.96 (d, J=8.8 Hz, 2H), 7.21 (d, J=5.3 Hz, 1H), 7.36 (d, J=8.8 Hz, 2H), 8.36 (d, J=5.3 Hz, 1H).

### Compounds 22 and 38: 1-acetyl-2-(4-ethoxyphenyl)-1H-pyrrolo[2,3-b]pyridin-6-yl acetate

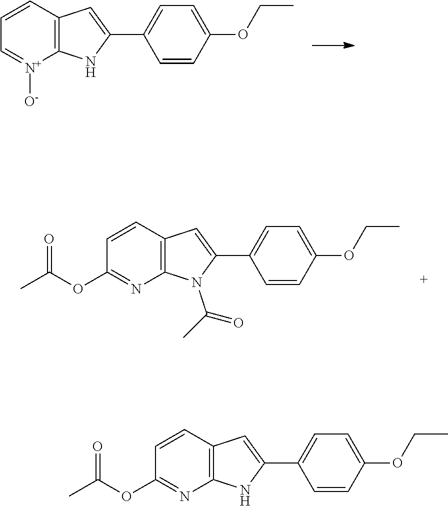

2-(4-ethoxyphenyl)-1H-pyrrolo[2,3-b]pyridine-7-oxide (compound 23) (100 mg, 0.39 mmol) was suspended in acetic anhydride (3 ml) and stirred at reflux for 16 h. The solution was cooled to r.t. and saturated NaHCO₃ was slowly added to quench excess reagent. The mixture was extracted with EtOAc (2×20 ml), washed with brine, dried over MgSO₄ and concentrated to an oil. Purification was performed by flash chromatography (70:30 PE:EtOAc) to afford a white solid (54 mg, 41%). ¹H NMR (300 MHz, CDCl₃) 7.91 (d, J=8.2 Hz, 1H), 7.32 (d, J=8.8 Hz, 2H), 6.96-6.91 (m, 3H), 6.52 (s, 1H), 4.07 (q, J=7.0 Hz, 2H), 2.99 (s, 3H), 2.38 (s, 3H), 1.44 (t, J=7.0 Hz, 3H).

Elution was continued (50:50 PE:EtOAc) to obtain a second product (compound 38, containing only an O-acetate) as a light yellow foam (23 mg, 20%). ¹H NMR (300 MHz, CDCl₃) δ 9.17 (s, 1H), 7.93 (dd, J=8.2, 0.7 Hz, 1H), 7.57 (d, J=8.8 Hz, 2H), 6.98 (d, J=8.8 Hz, 2H), 6.83 (d, J=8.2 Hz, 1H), 6.66 (d, J=2.2 Hz, 1H), 4.09 (q, J=7.0 Hz, 2H), 2.29 (s, 3H), 1.45 (t, J=7.0 Hz, 3H).

### Compound 23: 2-(4-ethoxyphenyl)-1H-pyrrolo[2,3-b]pyridine-7-oxide

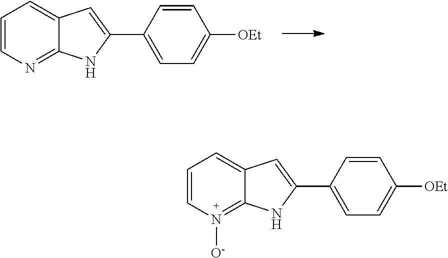

2-(4-ethoxyphenyl)-1H-pyrrolo[2,3-b]pyridine (2 g, 8.4 mmol) was suspended in a mixture of EtOAc (10 ml) and hexane (40 ml) under argon and cooled to 0° C. mCPBA (2.7 g, 12.6 mmol, 1.5 eq) was added in portion, the reaction was slowly warmed to r.t. and stirred for 12 h. The solvent was removed under vacuum and the residue was suspended in saturated K₂C₀₃ solution (50 ml), stirred vigorously for 30 min, filtered and washed with water to obtain a yellow solid that was dried under vacuum (1.5 g, 70%).

¹H-NMR (300 MHz, DMSO-d₆): 1.35 (t, J=7.1 Hz, 3H), 4.08 (q, J=7.1 Hz, 2H), 6.92 (s, 1H), 6.99-7.09 (m, 3H), 7.56 (d, J=7.1 Hz, 1H), 7.96 (d, J=8.8 Hz, 2H), 8.08 (d, J=6.3 Hz, 1H), 12.7 (bs, 1H).

### Compound 24: 2-(4-ethoxyphenyl)-N,N-diisopropyl-7H-pyrrolo[2,3-b]pyridine-7-carboxamide

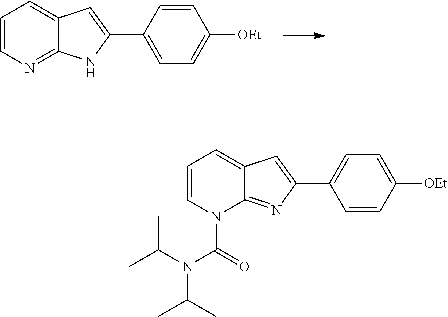 [SMILES: CCOc1ccc(-c2cc3cccnc3[nH]2)cc1 ; CCOc1ccc(-c2cc3cccn(C(=O)N(C(C)C)C(C)C)c-3n2)cc1]

2-(4-ethoxyphenyl)-1H-pyrrolo[2,3-b]pyridine (50 mg, 0.21 mmol) was dissolved in dry DMF (2 ml) and NaH (60% dispersion, 25 mg, 3 eq, 0.63 mmol) was added at r.t. After 15 min, N,N-diisopropylcarbamoyl chloride (41 mg, 1.2 eq, 0.25 mmol) was added in one portion and the mixture was stirred for 16 h. More electrophile was added (1.2 eq) and the reaction was stirred for 16 h before being quenched with water and extracted with EtOAc (2×10 ml). The extracts were washed with brine, dried over MgSO₄, filtered and concentrated. The crude product was purified by flash chromatography (90:10 to 80:20 PE:EtOAc) to afford a yellow solid (8 mg, 5%).

¹H-NMR (300 MHz, CDCl₃): 1.08 (d, J=6.6 Hz, 3H), 1.29 (d, J=6.6 Hz, 3H), 1.43 (t, J=7.0 Hz, 3H), 1.63 (d, J=6.7 Hz, 3H), 1.77 (d, J=6.7 Hz, 3H), 3.25-3.39 (m, 1H), 3.66-3.78 (m, 1H), 4.08 (q, J=7.0 Hz, 2H), 6.81 (dd, J=7.3, 6.4 Hz, 1H), 6.89 (s, 1H), 6.93 (d, J=8.9 Hz, 2H), 7.51 (dd, J=6.4, 1.1 Hz, 1H), 7.93 (dd, J=7.3, 1.1 Hz, 1H), 8.06 (d, J=8.9 Hz, 2H).

### Compound 25: benzyl 2-(4-ethoxyphenyl)-7H-pyrrolo[2,3-b]pyridine-7-carboxylate

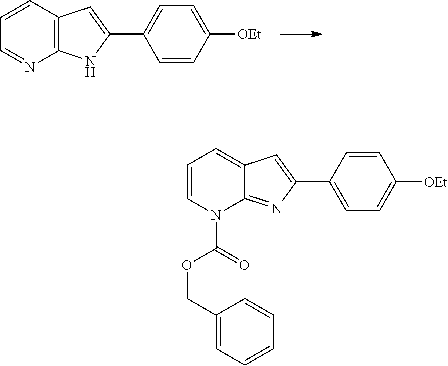 [SMILES: CCOc1ccc(-c2cc3cccnc3[nH]2)cc1 ; CCOc1ccc(-c2cc3cccn(C(=O)OCc4ccccc4)c-3n2)cc1]

2-(4-ethoxyphenyl)-1H-pyrrolo[2,3-b]pyridine (50 mg, 0.21 mmol) was dissolved in anhydrous DMF (2 ml) under argon, treated with powdered KOH (35 mg, 3 eq, 0.63 mmol) and stirred at r.t. for 15 min before adding benzyl chloroformate (36 μL, 1.2 eq, 0.25 mmol). The mixture was stirred 18 h at r.t., concentrated under vacuum and the residue was purified by flash chromatography (CH₂Cl₂) to afford a brown solid (13 mg, 17%).

¹H-NMR (300 MHz, CDCl₃): 1.44 (t, J=7.0 Hz, 3H), 4.10 (q, J=7.0 Hz, 2H), 5.93 (s, 2H), 6.74 (dd, J=7.3, 6.4 Hz, 1H), 6.92 (s, 1H), 6.97 (d, J=8.9 Hz, 2H), 7.31-7.50 (m, 6H), 7.92 (dd, J=7.4, 1.0 Hz, 1H), 8.11 (d, J=8.9 Hz, 2H).

### Compound 26: 7-benzyl-2-(4-ethoxyphenyl)-7H-pyrrolo[2,3-b]pyridine

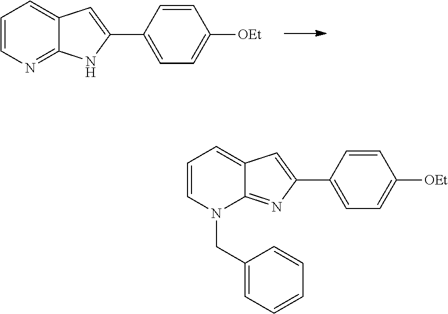 [SMILES: CCOc1ccc(-c2cc3cccnc3[nH]2)cc1 ; CCOc1ccc(-c2cc3cccn(Cc4ccccc4)c-3n2)cc1]

2-(4-ethoxyphenyl)-1H-pyrrolo[2,3-b]pyridine (50 mg, 0.21 mmol) was dissolved in anhydrous DMF (2 ml) under argon, treated with powdered KOH (35 mg, 3 eq, 0.63 mmol) and stirred at r.t. for 15 min before adding BnBr (30 μL, 1.2 eq, 0.25 mmol). The mixture was stirred 12 h at r.t., quenched with saturated NH₄Cl and extracted with CH₂Cl₂ (2×10 ml). The organic layer was washed with brine, dried over MgSO₄, filtered and concentrated. The crude product was purified by flash chromatography (60:40 PE:EtOAc) to obtain the N7-regioisomer as a yellow solid (26 mg, 38%).

¹H-NMR (300 MHz, CDCl₃): 1.45 (t, J=7.0 Hz, 3H), 4.10 (q, J=7.0 Hz, 2H), 5.92 (s, 2H), 6.72 (dd, J=7.4, 6.3 Hz, 1H), 6.93 (s, 1H), 6.95-7.03 (m, 2H), 7.31-7.51 (m, 6H), 7.91 (dd, J=7.4, 1.1 Hz, 1H), 8.07-8.18 (m, 2H).

### Compound 27: 2-(4-ethoxyphenyl)-7-(4-methoxybenzyl)-7H-pyrrolo[2,3-b]pyridine

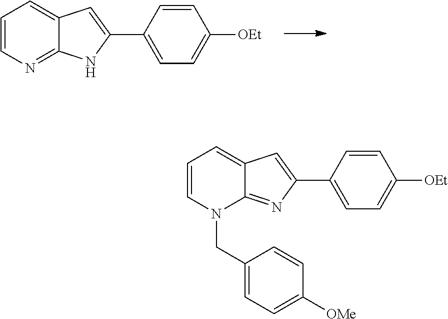 [SMILES: CCOc1ccc(-c2cc3cccnc3[nH]2)cc1 ; CCOc1ccc(-c2cc3cccn(Cc4ccc(OC)cc4)c-3n2)cc1]

2-(4-ethoxyphenyl)-1H-pyrrolo[2,3-b]pyridine (50 mg, 0.21 mmol) was dissolved in anhydrous DMF (2 ml) under argon, nBu₄NI (8 mg, 0.1 eq, 0.021 mmol) was added and the reaction was treated with NaH (42 mg, 5 eq, 1.05 mmol) and stirred at r.t. for 15 min before adding PMBCl (36 mg, 1.1 eq, 0.23 mmol). The mixture was stirred 12 h at r.t., quenched with water and extracted with EtOAc (2×10 ml). The organic layer was washed with brine, dried over MgSO₄, filtered and concentrated. The crude product was purified by flash chromatography (60:40 PE:EtOAc) to obtain the N7-regioisomer as a yellow oil (16 mg, 20%).

¹H-NMR (300 MHz, CDCl₃): 1.45 (t, J=7.0 Hz, 3H), 3.81 (s, 3H), 4.10 (q, J=7.0 Hz, 2H), 5.85 (s, 2H), 6.72 (dd, J=7.4, 6.3 Hz, 1H), 6.83-6.90 (m, 2H), 6.91 (s, 1H), 6.97 (d, J=8.9 Hz, 2H), 7.39 (dd, J=6.3, 1.1 Hz, 1H), 7.44 (d, J=8.7 Hz, 2H), 7.90 (dd, J 7.4, 1.1 Hz, 1H), 8.11 (d, J=8.9 Hz, 2H).

### Compound 28: (4-(7-benzyl-2-(4-ethoxyphenyl)-7H-pyrrolo[2,3-b]pyridin-4-yl)phenyl)(phenyl)methanone

### (4-(2-(4-ethoxyphenyl)-H-pyrrolo[2,3-b]pyridin-4-yl)phenyl)(phenyl)methanone

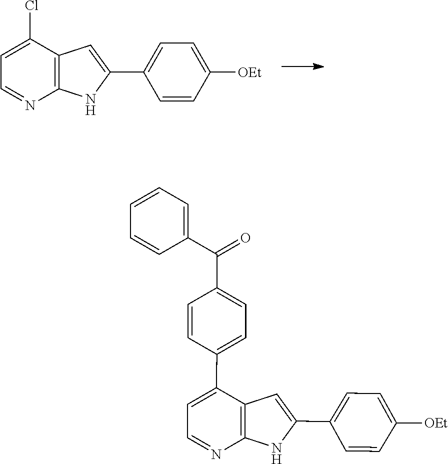 [SMILES: CCOc1ccc(-c2cc3c(Cl)ccnc3[nH]2)cc1 ; CCOc1ccc(-c2cc3c(-c4ccc(C(=O)c5ccccc5)cc4)ccnc3[nH]2)cc1]

4-chloro-2-(4-ethoxyphenyl)-1H-pyrrolo[2,3-b]pyridine (50 mg, 0.18 mmol) was charged into a vial with Pd(OAc)₂ (4 mg, 0.1 eq, 0.018 mmol), SPhos (15 mg, 0.2 eq, 0.036 mmol), K₂CO₃ (65 mg, 3 eq, 0.54 mmol) and (4-Benzoylphenyl)boronic acid (49 mg, 1.2 eq, 0.22 mmol). The vial was placed under argon and a mixture of dioxane (1.8 ml) and water (0.2 ml) was added before stirring 16 h at 100° C. After cooling the reaction was diluted with EtOAc and water, extracted with EtOAc (3×10 ml), washed with brine, dried over MgSO₄, filtered and concentrated. The crude product was purified by flash chromatography (70:30 PE:EtOAc) to afford a yellow solid (50 mg, 65%).

¹H-NMR (300 MHz, CDCl₃): 1.47 (t, J=7.0 Hz, 1H) 1.47 (t, J=7.0 Hz, 3H), 4.13 (q, J=7.0 Hz, 2H), 6.90 (s, 1H), 7.05 (d, J=8.8 Hz, 2H), 7.23 (d, J=5.1 Hz, 1H), 7.54 (t, J=7.4 Hz, 2H), 7.62 (d, J=5.0 Hz, 1H), 7.86-8.01 (m, 8H), 8.33 (d, J=5.0 Hz, 1H), 12.53 (s, 1H).

### Synthesis of Compound 28

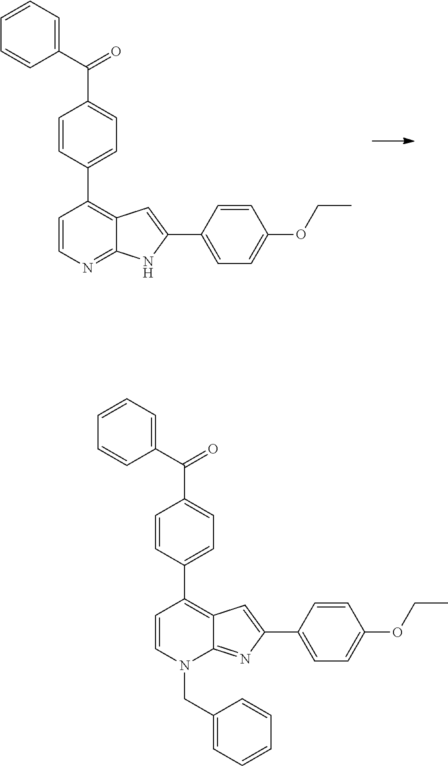 [SMILES: CCOc1ccc(-c2cc3c(-c4ccc(C(=O)c5ccccc5)cc4)ccnc3[nH]2)cc1 ; CCOc1ccc(-c2cc3c(-c4ccc(C(=O)c5ccccc5)cc4)ccn(Cc4ccccc4)c-3n2)cc1]

(4-(2-(4-ethoxyphenyl)-1H-pyrrolo[2,3-b]pyridin-4-yl)phenyl)(phenyl)methanone (30 mg, 0.07 mmol) was dissolved in dry DMF (1 ml) and NaH (60% dispersion, 12 mg, 3 eq, 0.22 mmol) was added. After 15 min, benzyl bromide (10 μL, 1.2 eq, 0.08 mmol) was added and the reaction was stirred at r.t. for 16 h. The mixture was quenched with water and extracted with EtOAc (2×10 ml). The organic layer was washed with brine, dried over MgSO₄, filtered and concentrated. The crude product was purified by flash chromatography (60:40 PE:EtOAc) to obtain the N7-regioisomer as an orange oil (12 mg, 32%).

¹H NMR (300 MHz, CDCl₃) 8.16 (d, J=8.8 Hz, 2H), 8.03-7.83 (m, 6H), 7.66-7.47 (m, 6H), 7.39-7.34 (m, 3H), 7.12 (s, 1H), 7.03-6.92 (m, 3H), 6.06 (s, 2H), 4.10 (q, J=7.0 Hz, 2H), 1.44 (t, J=7.0 Hz, 3H).

### Compound 29: tert-butyl 2-(4-hydroxyphenyl)-1H-indole-1-carboxylate

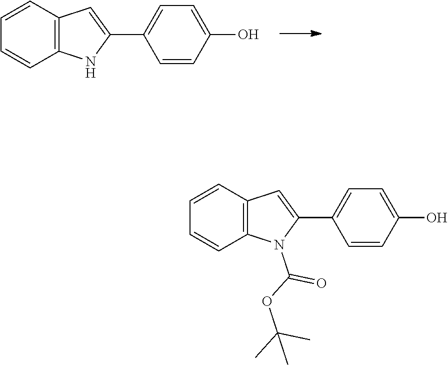 [SMILES: Oc1ccc(-c2cc3ccccc3[nH]2)cc1 ; CC(C)(C)OC(=O)n1c(-c2ccc(O)cc2)cc2ccccc21]

4-(1H-indol-2-yl)phenol (200 mg, 0.96 mmol, 1 eq) was placed under argon in a 25 ml flask, suspended in CH₂Cl₂ (10 ml) and cooled to 0° C. DMAP (12 mg, 0.09 mmol, 0.1 eq) was added, followed by Boc₂O (250 mg, 1.14 mmol, 1.2 eq) and the mixture was stirred while returning to r.t. for 4 h. Water was added and the mixture was extracted with EtOAc (2×20 ml), washed with water, dried over MgSO₄ and concentrated. Purification by flash chromatography (90:10 PE:EtOAc) afforded a brown solid (177 mg, 60%).

¹H NMR (300 MHz, CDCl₃) δ 8.33 (s, 1H), 7.72-7.62 (m, 3H), 7.42 (dd, J=8.0, 1.0 Hz, 1H), 7.33-7.11 (m, 4H), 6.81 (dd, J=2.2, 0.9 Hz, 1H), 1.61 (s, 9H).

### Compound 30: 4-(5-bromo-TH-indol-2-yl)phenol

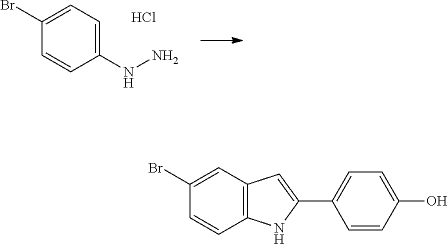 [SMILES: NNc1ccc(Br)cc1 ; Cl ; Oc1ccc(-c2cc3cc(Br)ccc3[nH]2)cc1]

4-bromophenyl hydrazine hydrochloride (5 g, 22.4 mmol, 1 eq) and 4-hydroxyacetophenone (3.2 g, 23.5 mmol, 1.05 eq) were dissolved in EtOH (50 ml) and heated to reflux for 4 h. The mixture was concentrated under vacuum to furnish the intermediate hydrazone, which was suspended in MsOH (40 ml) under argon. The suspension was heated to 90° C. for 7 h, cooled to r.t. and quenched by careful addition to saturated NaHCO₃. The product was extracted with a mixture of EtOAC:iPrOH (8:2, 3×100 ml), washed with brine, dried over MgSO₄ and concentrated to a black solid. The crude product was recrystallized in a mixture of toluene:EtOAc (95:5), cooled to −20° C. and filtered. This process was repeated on the concentrated mother liquor and both crops were combined to afford a grey solid (3.9 g, 60%).

¹H NMR (300 MHz, DMSO-d₆) δ 11.53 (s, 1H), 9.68 (s, 1H), 7.70-7.63 (m, 3H), 7.30 (d, J=8.5 Hz, 1H), 7.14 (dd, J=8.5, 2.0 Hz, 1H), 6.85 (d, J=8.7 Hz, 2H), 6.67 (d, J=2.1 Hz, 1H).

### Compound 31: 2-(4-(benzyloxy)phenyl)-1H-indole

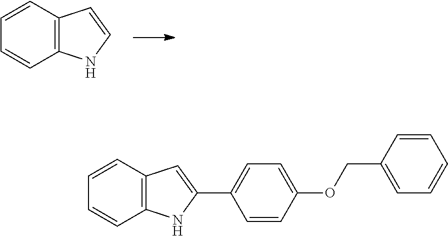 [SMILES: c1ccc2[nH]ccc2c1 ; c1ccc(COc2ccc(-c3cc4ccccc4[nH]3)cc2)cc1]

Indole (274 mg, 2.34 mmol, 1 eq) and 4-benzyloxyphenyl boronic acid (800 mg, 3.5 mmol, 1.5 eq) were placed in a 25 ml flask and dissolved in acetic acid (10 ml). Pd(OAc)₂ (52 mg, 0.23 mmol, 0.1 eq) was added and the mixture was vigorously stirred at r.t. under air for 20 h. The reaction was quenched by careful addition to saturated NaHCO₃ and extracted with EtOAc. The organic extracts were washed with brine, dried over MgSO₄ and concentrated to a brown oil. Purification by flash chromatography (100:0 to 90:10 CH₂Cl₂:MeOH) afforded a light brown solid (380 mg, 54%).

¹H NMR (300 MHz, DMSO-d₆): δ 11.40 (s, 1H), 7.79 (d, J=8.9 Hz, 2H), 7.50-7.34 (m, 7H), 7.11 (d, J=8.9 Hz, 2H), 7.08-7.02 (m, 1H), 7.00-6.94 (m, 1H), 6.76 (d, J=1.4 Hz, 1H), 5.17 (s, 2H).

### Compound 32: 2-(4-(benzyloxy)phenyl)-1H-indol-5-ol

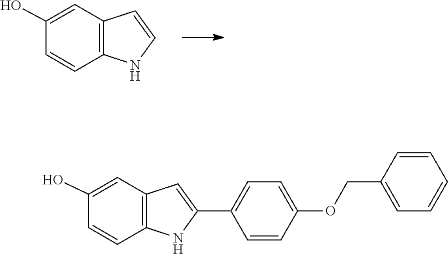 [SMILES: Oc1ccc2[nH]ccc2c1 ; Oc1ccc2[nH]c(-c3ccc(OCc4ccccc4)cc3)cc2c1]

5-hydroxyIndole (311 mg, 2.34 mmol, 1 eq) and 4-benzyloxyphenyl boronic acid (800 mg, 3.5 mmol, 1.5 eq) were placed in a 25 ml flask and dissolved in acetic acid (10 ml). Pd(OAc)₂ (52 mg, 0.23 mmol, 0.1 eq) was added and the mixture was vigorously stirred at r.t. under air for 20 h. The reaction was quenched by careful addition to saturated NaHCO₃ and extracted with a mixture of EtOAc:iPrOH (8:2). The organic extracts were washed with brine, dried over MgSO₄ and concentrated to a black solid. Purification by flash chromatography (100:0 to 80:20 CH₂Cl₂:EtOAc) afforded a light brown solid contaminated with impurities. The mixture was triturated in CH₂Cl₂ (5 ml) and filtered to obtain an off-white solid (115 mg, 16%).

¹H NMR (300 MHz, DMSO-d₆) δ 11.07 (d, J=2.2 Hz, 1H), 8.61 (s, 1H), 7.73 (d, J=8.7 Hz, 2H), 7.50-7.33 (m, 5H), 7.15 (d, J=8.6 Hz, 1H), 7.10 (d, J=8.7 Hz, 2H), 6.80 (d, J=2.3 Hz, 1H), 6.60-6.55 (m, 2H), 5.15 (s, 2H).

### Compound 33: 4-(1-methyl-1H-indol-2-yl)phenol

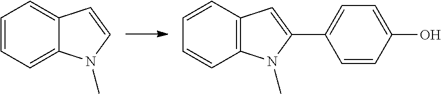

N-methylindole (190 μL, 1.52 mmol, 1 eq) was placed in a 25 ml flask along with Pd(OAc)₂ (17 mg, 0.08 mmol, 0.05 eq), Ag₂O (283 mg, 1.22 mmol, 0.8 eq), 4-iodophenol (669 mg, 3.04 mmol, 2 eq) and 2-nitrobenzoic acid (381 mg, 2.28 mmol, 1.5 eq) under argon. Dry DMF (8 ml) was added and the reaction was stirred at r.t. for 16 h. The mixture was filtered on Celite®, washing with EtOAc and brine was added. The aqueous phase was extracted with EtOAc (2×30 ml), the organic extracts were washed with brine, dried over MgSO₄ and concentrated. Purification by flash chromatography (95:5 to 80:20 PE:EtOAc) afforded a beige waxy solid (139 mg, 36%).

¹H NMR (300 MHz, CDCl₃) δ 7.65-7.60 (m, 1H), 7.38 (d, J=8.6 Hz, 2H), 7.26-7.20 (m, 1H), 7.16-7.10 (m, 1H), 6.93 (d, J=8.6 Hz, 2H), 6.50 (d, J=0.8 Hz, 1H), 3.72 (s, 3H).

### Compound 34: 2-(4-ethoxyphenyl)-1H-pyrrolo[2,3-c]pyridin-1-yl diethylcarbamate

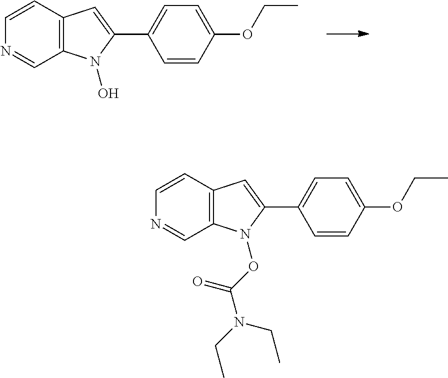 [SMILES: CCOc1ccc(-c2cc3ccncc3n2O)cc1 ; CCOc1ccc(-c2cc3ccncc3n2OC(=O)N(CC)CC)cc1]

2-(4-ethoxyphenyl)-1H-pyrrolo[2,3-c]pyridin-1-ol (125 mg, 0.5 mmol, 1 eq) was suspended in dry CH₂Cl₂ (50 ml) under argon and cooled to 0° C. DIPEA (130 μL, 0.75 mmol, 1.5 eq) was added, followed by N,N-diethylcarbamoyl chloride (75 μL, 0.6 mmol, 1.2 eq) and the mixture was stirred for 16 h while returning to r.t. The reaction was quenched by addition of water extracted with CH₂Cl₂ (2×20 ml), washed with water, dried over MgSO₄ and concentrated. Purification by flash chromatography (80:20 to 50:50 EtOAc:CH₂Cl₂) afforded a red oil that solidified on standing (106 mg, 61%).

¹H NMR (300 MHz, CDCl₃) δ 8.58 (s, 1H), 8.22 (d, J=5.5 Hz, 1H), 7.51 (d, J=8.8 Hz, 2H), 7.39 (dd, J=5.5, 1.0 Hz, 1H), 6.90 (d, J=8.8 Hz, 2H), 6.44 (d, J=0.8 Hz, 1H), 4.01 (q, J=7.0 Hz, 2H), 3.34 (q, J=7.3 Hz, 2H), 3.24 (q, J=7.3 Hz, 2H), 1.37 (t, J=7.0 Hz, 3H), 1.14 (t, J=7.3 Hz, 3H), 1.07 (t, J=7.3 Hz, 3H).

### Compound 35: 2-(4-ethoxyphenyl)-1H-pyrrolo[2,3-b]pyridine-6-carbonitrile

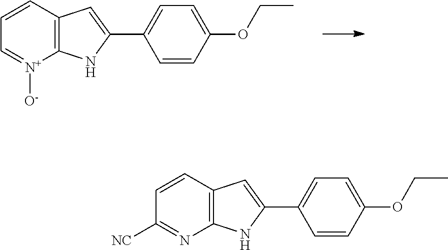 [SMILES: CCOc1ccc(-c2cc3ccc[n+]([O-])c3[nH]2)cc1 ; CCOc1ccc(-c2cc3ccc(C#N)nc3[nH]2)cc1]

2-(4-ethoxyphenyl)-1H-pyrrolo[2,3-b]pyridine-7-oxide (190 mg, 0.75 mmol, 1 eq) was suspended in dry MeCN (5 ml) in a 25 ml pressure tube placed under argon. Dimethyl sulfate (105 mg, 0.83 mmol, 1.1 eq) was added and the mixture was heated to 60° C. overnight. The next day, more dimethyl sulfate was added (1 eq) and the mixture was heated for 16 h. After cooling to r.t., KCN (147 mg, 2.25 mmol, 3 eq) and saturated NH₄Cl (5 ml) were added and the biphasic mixture was vigorously stirred at 50° C. for 48 h. The reaction was quenched with saturated NaHCO₃, extracted with EtOAc (2×20 ml), washed with brine, dried over MgSO₄ and concentrated. Purification was performed by flash chromatography (100:0 to 90:10 CH₂Cl₂:EtOAc) to afford a light orange solid (62 mg, 32%).

¹H NMR (300 MHz, DMSO-d₆) δ 12.61 (s, 1H), 8.09 (d, J=8.1 Hz 1H), 7.94 (d, J=8.7 Hz, 2H), 7.60 (d, J=8.1 Hz, 1H), 7.07 (d, J=8.7 Hz, 2H), 7.00 (s, 1H), 4.11 (q, J=7.3 Hz, 2H), 1.35 (t, J=7.3 Hz, 2H).

### Compound 36: 2-(4-ethoxyphenyl)-1H-pyrrolo[2,3-b]pyridin-6-amine

### 3-((4-ethoxyphenyl)ethynyl)pyridine-2,6-diamine

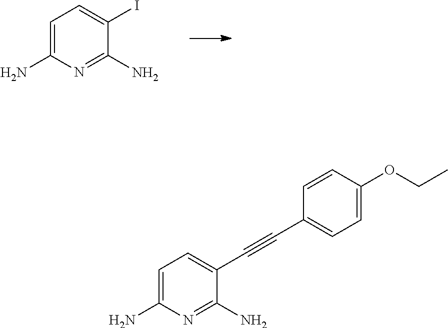 [SMILES: Nc1ccc(I)c(N)n1 ; CCOc1ccc(C#Cc2ccc(N)nc2N)cc1]

3-iodopyridine-2,6-diamine (synthesized from 2,6-diaminopyridine according to WO2019149965) (2 g, 8.51 mmol, 1 eq) was placed under argon and dissolved in anhydrous THF (20 ml). Et₃N (5.9 ml, 5 eq, 42.5 mmol) and 1-ethoxy-4-ethynylbenzene (1.49 ml, 1.2 eq, 10.2 mmol) were added, followed by CuI (40 mg, 0.025 eq, 0.21 mmol) and PdCl₂(PPh₃)₂ (150 mg, 0.025 eq, 0.21 mmol). The dark brown mixture was stirred at r.t. for 16 h, before water (20 ml) and CH₂Cl₂ (20 ml) were added. The aqueous layer was extracted with CH₂Cl₂ and the organic extracts were washed with water and brine, dried over MgSO₄ and concentrated. Purification by flash chromatography (CH₂Cl₂:EtOAc 90:10 to 0:100) afforded the desired compound as a brown solid (1.95 g, 90%).

¹H NMR (300 MHz, DMSO-d₆) δ 7.43 (d, J=8.8 Hz, 2H), 7.19 (d, J=8.2 Hz, 1H), 6.91 (d, J=8.8 Hz, 2H), 5.88 (s, 2H), 5.83-5.61 (m, 3H), 4.04 (q, J=7.0 Hz, 2H), 1.33 (t, J=7.0 Hz, 3H).

### Synthesis of Compound 36

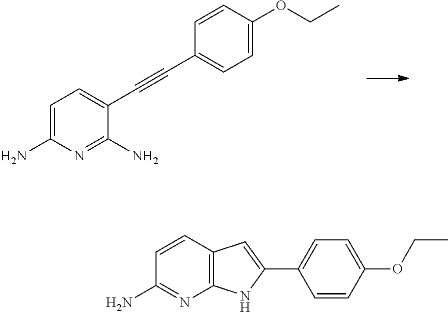 [SMILES: CCOc1ccc(C#Cc2ccc(N)nc2N)cc1 ; CCOc1ccc(-c2cc3ccc(N)nc3[nH]2)cc1]

3-((4-ethoxyphenyl)ethynyl)pyridine-2,6-diamine (200 mg, 0.79 mmol, 1 eq) was dissolved in dry DMSO (8 ml), ^{t}BuOK (220 mg, 1.97 mmol, 2.5 eq) was added in one portion and the brown mixture was heated to 65° C. for 16 h. The reaction was quenched with saturated NH₄Cl and extracted with a mixture of EtOAc and iPrOH (8:2, 3×50 ml). The organic extracts were dried over MgSO₄ and concentrated to a black solid. Purification by flash chromatography (98:2 to 95:5 CH₂Cl₂:MeOH) afforded an impure product that was further purified by suspension in Et₂O/EtOAc and sonication. The resulting red solid was filtered, washed with Et₂O and dried under vacuum (25 mg, 12%).

¹H NMR (300 MHz, DMSO-d₆) δ 11.23 (s, 1H), 7.69 (d, J=8.3 Hz, 2H), 7.50 (d, J=8.6 Hz, 1H), 6.92 (d, J=8.3 Hz, 2H), 6.50 (s, 1H), 6.25 (d, J=8.3 Hz, 1H), 5.61 (s, 2H), 4.05 (q, J=6.9 Hz, 4H), 1.34 (t, J=6.9 Hz, 5H).

### Compound 37: 4-(6-chloro-1H-pyrrolo[2,3-b]pyridin-2-yl)phenol

### 6-chloro-2-(4-ethoxyphenyl)-1H-pyrrolo[2,3-b]pyridine

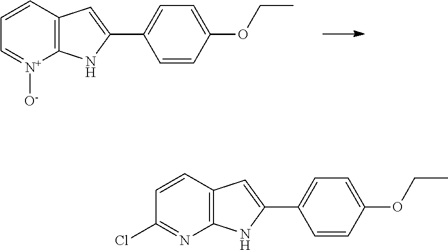 [SMILES: CCOc1ccc(-c2cc3ccc[n+]([O-])c3[nH]2)cc1 ; CCOc1ccc(-c2cc3ccc(Cl)nc3[nH]2)cc1]

2-(4-ethoxyphenyl)-1H-pyrrolo[2,3-b]pyridine-7-oxide (4 g, 16 mmol, 1 eq) was suspended in dry THF (60 ml) under argon and cooled to 0° C. HMDS (2.6 g, 16 mmol, 1 eq) was added in one portion, followed by dropwise addition of ethyl chloroformate (4.34 g, 40 mmol, 2.5 eq). The reaction was stirred for 16 h while returning to r.t., recooled to 0° C. and more HMDS (0.5 eq) and ethyl chloroformate (1.25 eq) were added. After 16 h, the mixture was quenched with saturated NaHCO₃ and THF was removed under vacuum. The resulting red oil was dissolved in MeOH (50 ml) and NaOH (2N, 50 ml) was added. After 5 h, saturated NH₄Cl was added and MeOH was removed under vacuum. The mixture was extracted with EtOAc (3×100 ml), washed with brine, dried over MgSO₄ and concentrated to give a mixture of isomers. Purification was performed by flash chromatography (90:10 EtOAc:CH₂Cl₂) to afford the 6-chloroazaindole isomer as a tan solid (1.4 g, 36%).

¹H NMR (300 MHz, DMSO-d₆) δ 12.28 (s, 1H), 7.91 (d, J=8.2 Hz, 1H), 7.83 (d, J=8.8 Hz, 2H), 7.09 (d, J=8.2 Hz, 1H), 7.00 (d, J=8.8 Hz, 2H), 6.79 (d, J=2.1 Hz, 1H), 4.05 (q, J=7.0 Hz, 2H), 1.33 (t, J=7.0 Hz, 3H).

### Synthesis of Compound 37

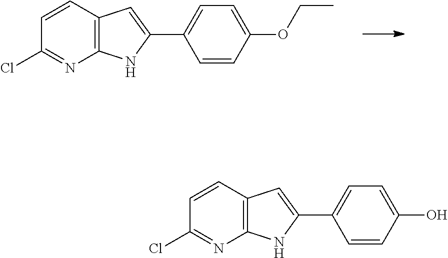 [SMILES: CCOc1ccc(-c2cc3ccc(Cl)nc3[nH]2)cc1 ; Oc1ccc(-c2cc3ccc(Cl)nc3[nH]2)cc1]

6-chloro-2-(4-ethoxyphenyl)-1H-pyrrolo[2,3-b]pyridine (150 mg, 0.55 mmol, 1 eq) was suspended in anhydrous CH₂Cl₂ (10 ml) under argon and cooled to −78° C. Boron tribromide (1M solution in CH₂Cl₂, 1.65 ml, 1.65 mmol, 3 eq) was added dropwise and the dark brown mixture was stirred 6 h while slowly warming to r.t. The reaction was carefully quenched with saturated NaHCO₃ solution (10 ml) and cooled to 0° C. before addition of 2M NaOH (10 ml). The solution was stirred for 10 min before being loaded in a separating funnel and washed with dichloromethane. The aqueous phase was then neutralized with dropwise addition of 6M HCl under stirring. The solid was filtered, washed with saturated NaHCO₃, water and dried under vacuum to give a brown powder of satisfactory purity (130 mg, quantitative).

¹H-NMR (300 MHz, DMSO-d₆): δ 12.18 (s, 1H), 9.76 (s, 1H), 7.92 (d, J=8.1 Hz, 1H), 7.73 (d, J=8.7 Hz, 2H), 7.08 (d, J=8.1 Hz, 1H), 6.86 (d, J=8.7 Hz, 2H), 6.76 (d, J=1.7 Hz, 1H).

### Compounds 39 and 40: 4-(6-(6-methoxypyridin-3-yl)-1H-pyrrolo[2,3-b]pyridin-2-yl)phenol and 5-(2-(4-hydroxyphenyl)-1H-pyrrolo[2,3-b]pyridin-6-yl)pyridin-2-ol

### 2-(4-ethoxyphenyl)-6-(6-methoxypyridin-3-yl)-1H-pyrrolo[2,3-b]pyridine

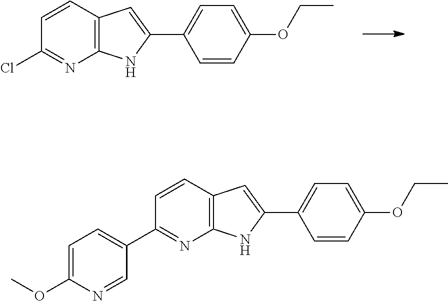

6-chloro-2-(4-ethoxyphenyl)-1H-pyrrolo[2,3-b]pyridine (50 mg, 0.20 mmol, 1 eq) was placed in a 25 ml schlenk flask under argon along with K₂CO₃ (83 mg, 0.6 mmol, 3 eq), SPhos (16 mg, 0.04 mmol, 0.2 eq), Pd(OAc)₂ (4.5 mg, 0.020 mmol, 0.1 eq) and (6-methoxypyridin-3-yl)boronic acid (43 mg, 0.28 mmol, 1.4 eq). Dioxane (4.5 ml) and water (0.5 ml) were added and the mixture was heated to 100° C. for 4 h. After cooling to r.t., water was added and the mixture was extracted with EtOAc (2×10 ml), washed with brine, dried over MgSO₄ and concentrated. Purification by flash chromatography (100:0 to 70:30 CH₂Cl₂:EtOAc) afforded a brown solid (44 mg, 63%).

¹H NMR (300 MHz, DMSO-d₆) δ 12.12 (s, 1H), 8.88 (d, J=2.5 Hz, 1H), 8.39 (dd, J=8.7, 2.5 Hz, 1H), 7.96 (d, J=8.1 Hz, 1H), 7.89 (d, J=8.8 Hz, 2H), 7.63 (d, J=8.1 Hz, 1H), 7.02 (d, J=8.8 Hz, 2H), 6.94 (d, J=8.7 Hz, 1H), 6.82 (s, 1H), 4.09 (q, J=7.0 Hz, 2H), 3.92 (s, 3H), 1.35 (t, J=7.0 Hz, 3H).

### Synthesis of Compounds 39 and 40

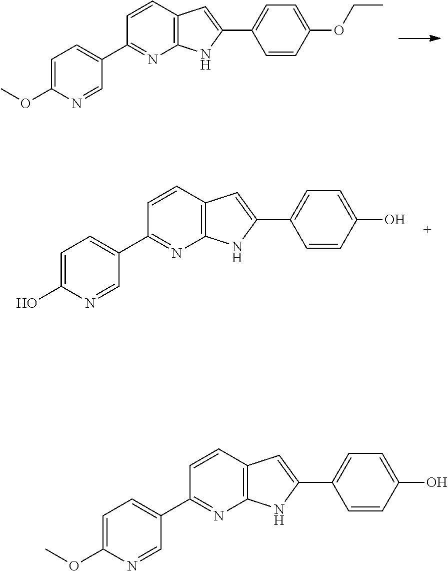

2-(4-ethoxyphenyl)-6-(6-methoxypyridin-3-yl)-1H-pyrrolo[2,3-b]pyridine (24 mg, 0.07 mmol, 1 eq) was dissolved in dry CH₂Cl₂ (5 ml) under argon and cooled to 0° C. AlCl₃ (55 mg, 0.28 mmol, 4 eq) was added and the mixture was stirred for 16 h while returning to r.t. The next day, more AlCl₃ (4 eq) was added and the mixture was heated to reflux for 8 h and stirred at r.t. overnight. The reaction was quenched with saturated NaHCO₃, extracted with EtOAc (2×50 ml), washed with brine, dried under MgSO₄ and concentrated. Purification by flash chromatography afforded two products: the first one eluted with 98:2 CH₂Cl₂:MeOH to give a yellow solid and was identified as the monoether 39 (8 mg, 36%).

¹H NMR (300 MHz, Methanol-d₄) δ 8.80 (d, J=2.5 Hz, 1H), 8.33 (dd, J=8.7, 2.5 Hz, 1H), 7.89 (d, J=8.2 Hz, 1H), 7.68 (d, J=8.7 Hz, 2H), 7.48 (d, J=8.2 Hz, 2H), 6.90-6.86 (m, 3H), 6.65 (s, 1H), 3.96 (s, 3H).

Elution was continued with 95:5 CH₂Cl₂:MeOH to obtain an orange solid, identified as the completely dealkyated product 40 (7 mg, 33%). ¹H NMR (300 MHz, DMSO-d₆) δ 11.93 (s, 1H), 11.84 (s, 2H), 9.71 (s, 1H), 8.23 (dd, J=9.6, 2.7 Hz, 1H), 8.09 (s, 1H), 7.88 (d, J=8.2 Hz, 1H), 7.76 (d, J=8.6 Hz, 2H), 7.47 (d, J=8.2 Hz, 1H), 6.85 (d, J=8.7 Hz, 2H), 6.71 (d, J=2.1 Hz, 1H), 6.47 (d, J=9.6 Hz, 1H).

### Compound 41: 2-(4-ethoxyphenyl)-6-(1H-pyrazol-4-yl)-1H-pyrrolo[2,3-b]pyridine

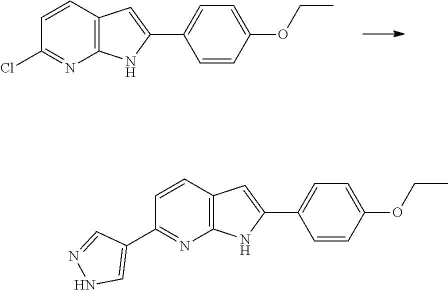 [SMILES: CCOc1ccc(-c2cc3ccc(Cl)nc3[nH]2)cc1 ; CCOc1ccc(-c2cc3ccc(-c4cn[nH]c4)nc3[nH]2)cc1]

6-chloro-2-(4-ethoxyphenyl)-1H-pyrrolo[2,3-b]pyridine (50 mg, 0.20 mmol, 1 eq) was placed in a 25 ml schlenk flask under argon along with K₂CO₃ (83 mg, 0.6 mmol, 3 eq), Dppf (22 mg, 0.04 mmol, 0.2 eq), Pd(OAc)₂ (4.5 mg, 0.020 mmol, 0.1 eq) and 4-(4,4,5,5-tetramethyl-1,3,2-dioxaborolan-2-yl)-1H-pyrazole (54 mg, 0.28 mmol, 1.4 eq). Dioxane (4.5 ml) and water (0.5 ml) were added and the mixture was heated to 100° C. for 4 h. After cooling to r.t., water was added and the mixture was extracted with EtOAc (2×10 ml), washed with brine, dried over MgSO₄ and concentrated. Purification by flash chromatography (100:0 to 92:8 CH₂Cl₂:MeOH) afforded a brown solid (25 mg, 41%).

¹H NMR (300 MHz, DMSO-d₆) δ 12.97 (s, 1H), 12.00 (s, 1H), 8.26 (s, 1H), 8.05 (s, 1H), 7.88-7.83 (m, 3H), 7.40 (d, J=8.1 Hz, 1H), 7.01 (d, J=8.9 Hz, 2H), 6.75 (d, J=2.1 Hz, 1H), 4.09 (q, J=7.0 Hz, 2H), 1.36 (t, J=7.0 Hz, 3H).

### Compound 43: 2-(4-ethoxyphenyl)-N-phenyl-1-(phenylsulfonyl)-1H-pyrrolo[2,3-b]pyridin-4-amine

### 4-chloro-2-(4-ethoxyphenyl)-1-(phenylsulfonyl)-1H-pyrrolo[2,3-b]pyridine

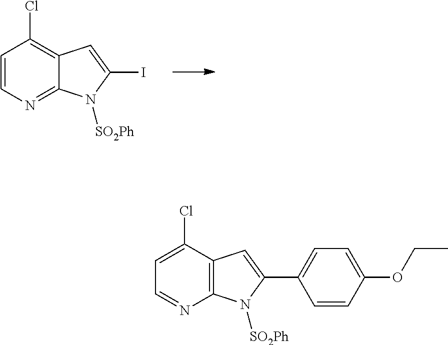

4-chloro-2-iodo-1-(phenylsulfonyl)-1H-pyrrolo[2,3-b]pyridine (synthesized from 4-chloro-1H-pyrrolo[2,3-b]pyridine according to WO2019058132) (1.5 g, 3.5 mmol, 1 eq) was placed in a 100 ml schlenk flask under argon along with 4-ethoxyphenyl boronic acid (0.7 g, 4.2 mmol, 1.2 eq), Na₂CO₃ (1.1 g, 10.5 mmol, 3 eq) and Pd(PPh₃)₂Cl₂ (0.12 g, 0.18 mmol, 0.05 eq). A mixture of dioxane (50 ml) and water (15 ml) was added and the reaction was heated to 70° C. for 4 h. After cooling to r.t., water was added and the mixture was extracted with EtOAc (2×50 ml), washed with brine, dried over MgSO₄ and concentrated. Purification by flash chromatography (90:10 to 70:30 PE:EtOAc) afforded an orange foam (1.21 g, 84%).

¹H NMR (300 MHz, CDCl₃) δ 8.34 (d, J=5.3 Hz, 1H), 7.86-7.83 (m, 2H), 7.53-7.36 (m, 5H), 7.20 (d, J=5.3 Hz, 1H), 6.97 (d, J=8.7 Hz, 2H), 6.56 (s, 1H), 4.12 (q, J=7.0 Hz, 2H), 1.47 (t, J=7.0 Hz, 3H).

### Synthesis of Compound 43

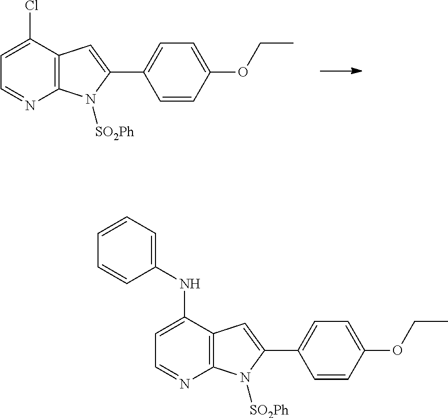

4-chloro-2-(4-ethoxyphenyl)-1-(phenylsulfonyl)-1H-pyrrolo[2,3-b]pyridine (100 mg, 0.24 mmol, 1 eq) was placed in a 25 ml schlenk flask under argon along with Cs₂CO₃ (156 mg, 0.48 mmol, 2 eq), XPhos (23 mg, 0.048 mmol, 0.2 eq) and Pd₂dba₃ (22 mg, 0.024 mmol, 0.1 eq). Dioxane (10 ml) was added, followed by aniline (32 mg, 0.34 mmol, 1.4 eq) and the mixture was heated to 100° C. for 4 h. After cooling to r.t., water was added and the mixture was extracted with EtOAc (2×50 ml), washed with brine, dried over MgSO₄ and concentrated. Purification by flash chromatography (90:10 to 70:30 PE:EtOAc) afforded a yellow oil (87 mg, 79%).

¹H NMR (300 MHz, CDCl₃) δ 8.20 (d, J=5.6 Hz, 1H), 7.88 (d, J=7.8 Hz, 2H), 7.58-7.33 (m, 7H), 7.26-7.12 (m, 3H), 6.97 (d, J=7.8 Hz, 2H), 6.85 (d, J=5.6 Hz, 1H), 6.37 (d, J=0.8 Hz, 1H), 6.15 (s, 1H), 4.14 (q, J=7.0 Hz, 2H), 1.49 (t, J=7.0 Hz, 3H).

### Compound 44: 2-(4-ethoxyphenyl)-N-methyl-N-phenyl-1H-pyrrolo[2,3-b]pyridin-4-amine

### 2-(4-ethoxyphenyl)-N-methyl-N-phenyl-1-(phenylsulfonyl)-1H-pyrrolo[2,3-b]pyridin-4-amine

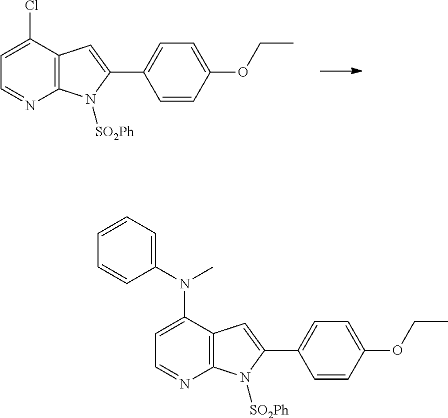

4-chloro-2-(4-ethoxyphenyl)-1-(phenylsulfonyl)-1H-pyrrolo[2,3-b]pyridine (100 mg, 0.24 mmol, 1 eq) was placed in a 25 ml schlenk flask under argon along with Cs₂CO₃ (156 mg, 0.48 mmol, 2 eq), XPhos (23 mg, 0.048 mmol, 0.2 eq) and Pd₂dba₃ (22 mg, 0.024 mmol, 0.1 eq). Dioxane (10 ml) was added, followed by N-methylaniline (36 mg, 0.34 mmol, 1.4 eq) and the mixture was heated to 100° C. for 4 h. After cooling to r.t., water was added and the mixture was extracted with EtOAc (2×50 ml), washed with brine, dried over MgSO₄ and concentrated. Purification by flash chromatography (90:10 to 70:30 PE:EtOAc) afforded a yellow oil (67 mg, 55%).

### Synthesis of Compound 44

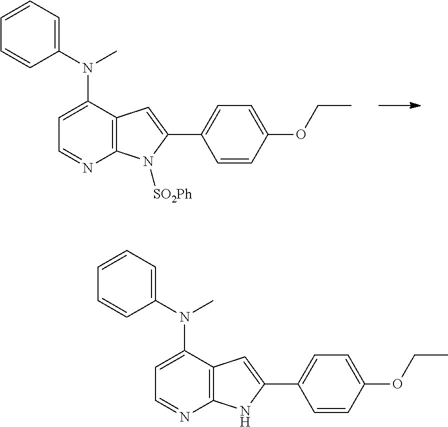

2-(4-ethoxyphenyl)-N-methyl-N-phenyl-1-(phenylsulfonyl)-1H-pyrrolo[2,3-b]pyridin-4-amine (66 mg, 0.14 mmol, 1 eq) was dissolved in a mixture of THF (5 ml) and MeOH (5 ml) under argon. NaOH (140 μL, 2M, 2 eq) was added and the reaction was heated to 55° C. overnight. After cooling to r.t., saturated NH₄Cl (10 ml) was added and the product was extracted with EtOAc (3×10 ml), washed with brine, dried over MgSO₄ and concentrated. Purification by flash chromatography (80:20 to 0:100 CH₂Cl₂:EtOAc) afforded a yellow solid (17 mg, 35%).

¹H NMR (300 MHz, DMSO-d₆) δ 11.75 (s, 1H), 7.99 (d, J=5.5 Hz, 1H), 7.50 (d, J=8.8 Hz, 2H), 7.42-7.37 (m, 2H), 7.26-7.16 (m, 3H), 6.93 (d, J=8.8 Hz, 2H), 6.55 (d, J=5.5 Hz, 1H), 5.50 (d, J=2.3 Hz, 1H), 4.03 (q, J=7.0 Hz, 2H), 3.46 (s, 3H), 1.32 (t, J=7.0 Hz, 3H).

II. Biological Activity of the Compounds According to the Invention

The activity of the compounds against ferroptosis was evaluated in vitro on cellular models of pathologies. Ferroptosis is involved in several pathologies or dysfunctions of the organism, and notably in acute kidney injury associated with ischemia-reperfusion or resulting from the administration of cisplatin during anti-cancer chemotherapy (in particular during the treatment of head and neck (ENT), lung, endometrial or bladder cancers). Ferroptosis is also involved in the physiopathology of neurodegenerative diseases, such as Parkinson's disease, or in neurological disorders associated with excitotoxicity, such as trauma and stroke.

II.1. Neurotoxicity and Excitotoxicity Assay

Model

The excitotoxicity model used was the mouse hippocampal neuronal HT22 cells treated with glutamate [Dixon et al., Cell, 2012, 149(5), 1060-1072]. Ferroptosis and oxidative stress are both associated in this glutamate-induced cell death [Chu et al., Neural Regen. Res., 2020, 15(3), 528-536]. Phenotypic screening was also carried out with additional cell death inducers, erastin and RSL3 ((1S,3R)-Ras-selective lethal), in HT22 and SH-SY5Y (human neuroblastoma cells). Erastin and RSL3 are well described molecules, used as chemical tools for the study of ferroptosis regulation and neuronal pathologies [Lewerenz et al., Front. Neurosci., 2018, 12: 214; Reichert et al., Int. J. Mol. Sci., 2020, 21, 8765].

Material and Methods

Mouse hippocampal neuronal cell line (HT22, ATCC) and human neuroblastoma cell line (SH-SY5Y, ATCC) were grown in Dulbecco's Modified Eagle Medium (DMEM, Gibco) supplemented with 10% fetal bovine serum (FBS, Gibco) at 37° C. under 5% CO₂ atmosphere. Neurotoxicity was induced by erastin or RSL3 and excitotoxicity was induced by glutamate. Cells were seeded in 96-well plates at 5,000 and 10,000 cells/well for HT22 and SH-SY5Y cell lines, respectively. Cells were treated with 0.5-1 μM Erastin (HT22 cells) or 10 μM Erastin (SH-SY5Y cells) or 5-10 mM glutamate (HT22 cells) or 0.5 μM RSL3 (HT22 cells) or 5 μM RSL3 (SH-SY5Y cells) for 24 h in presence of increasing concentrations of inhibitors. Cells treated with DMSO only were used as controls. Cell viability was assessed by MTS assay (CellTiter 96 AQueous One Solution Cell Proliferation assay, Promega).

Results

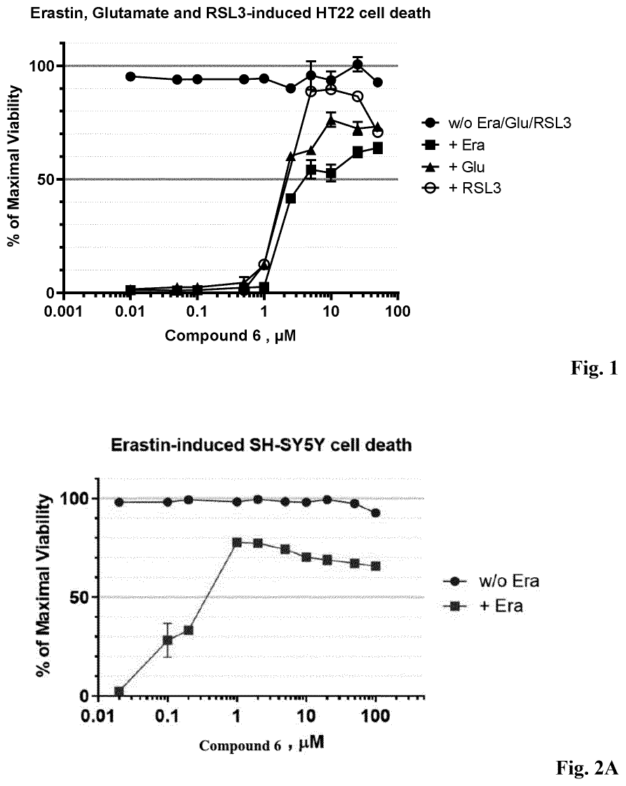

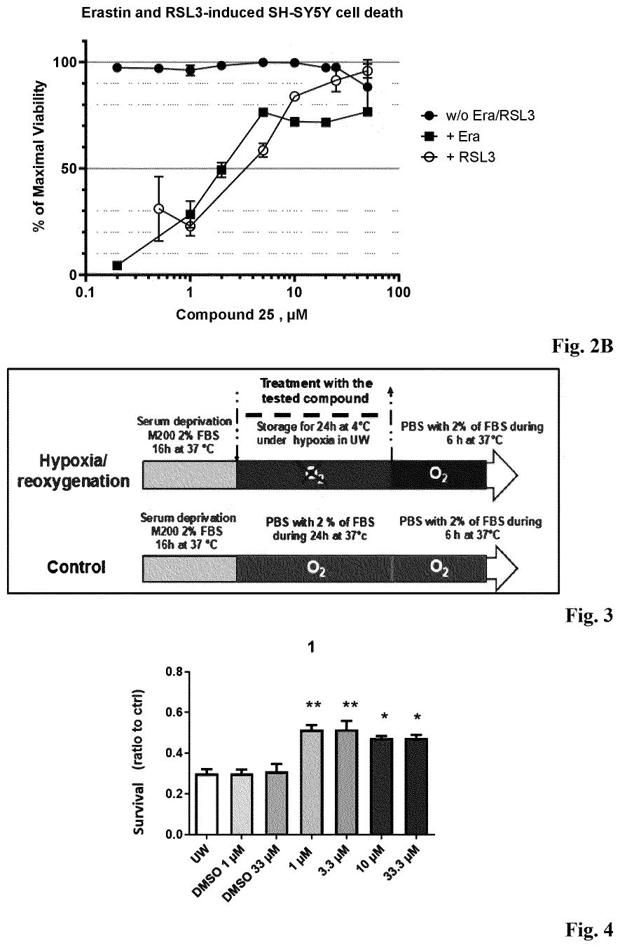

In FIGS. 1, 2A and 2B, an inhibitor was added to HT22 (FIG. 1: compound 6) or SH-SY5Y (FIG. 2A: compound 6; FIG. 2B: compound 25) cells in the presence or not of cell death inducers (erastin, glutamate or RSL3). The curves showed the cell viability measured after 24 hours of treatment and the addition of tetrazolium salt (CellTiter 96 AQueous One Solution Cell Proliferation Assay (MTS), #G3582, Promega). The results obtained without cell death inducers showed the potential cytotoxic effect of the molecule alone. It can be noticed that compounds 6 and 25 have no effect on cell viability at the doses tested (maximum 50 or 100 μM). The results obtained in the presence of a ferroptosis inducer showed that increasing concentrations of compound 6 increased cell viability until reaching almost total inhibition at 10 μM on HT22 cells (EC₅₀=2.05 μM in the presence of 1 μM of erastin, 1.64 μM in the presence of 10 mM glutamate and 2.31 μM in the presence of 0.5 μM RSL3) or 1 μM on SH-SY5Y cells (EC₅₀=0.18 μM). Similarly, increasing concentrations of compound 25 increased cell viability until reaching almost total inhibition at 10 μM on SH-SY5Y cells (EC₅₀=1.4 μM in the presence of 10 μM of erastin and 5.25 μM in the presence of 5 μM RSL3). EC₅₀ values for compounds 1-28 were calculated from dose-response curves using Graphpad Prism software. The results are reported in Table 1 hereafter.

TABLE 1
EC₅₀ calculated from results obtained using phenotypic assays HT-22 +
glutamate, erastin or RSL3 and SH-SY5Y + erastin or RSL3

| | EC₅₀ | | EC₅₀ | | EC₅₀ | | EC₅₀ | | EC₅₀ | |
|---|---|---|---|---|---|---|---|---|---|---|
| | HT22 | | HT22 | | HT22 | | SH-SY5Y | | SH-SY5Y | |
| Compound | Glutamate | | Erastin | | RSL3 | | Erastin | | RSL3 | |
| 1 | 2.55 | μM | 2.9 | μM | 3.27 | μM | 1.06 | μM | ND | |
| 2 | 2.66 | μM | 7.92 | μM | 46 | μM | 2.9 | μM | 7.43 | μM |
| 3 | 1.13 | μM | 0.92 | μM | 0.32 | μM | ND | | ND | |
| 4 | >25 | μM | >25 | μM | 27.8 | μM | 7.66 | μM | 5.42 | μM |
| 5 | 3.6 | μM | 3.23 | μM | 2.79 | μM | 0.52 | μM | ND | |
| 6 | 1.64 | μM | 2.05 | μM | 2.31 | μM | 0.18 | μM | ND | |
| 7 | >25 | μM | >25 | μM | 14.55 | μM | 12.4 | μM | 1.36 | μM |
| 8 | >25 | μM | >25 | μM | >50 | μM | 21.52 | μM | 5.98 | μM |
| 9 | >25 | μM | >25 | μM | 1.43 | μM | 10.64 | μM | ND | |
| 10 | >25 | μM | >25 | μM | >50 | μM | 13.7 | μM | ND | |
| 11 | 0.14 | μM | 0.32 | μM | 1.16 | μM | 0.15 | μM | ND | |
| 12 | 0.50 | μM | 0.91 | μM | 0.94 | μM | 0.43 | μM | ND | |
| 13 | 0.92 | μM | 3.62 | μM | 44.06 | μM | 1.11 | μM | 9.25 | μM |
| 14 | 0.55 | μM | 1.52 | μM | 0.76 | μM | 1.11 | μM | ND | |
| 15 | 0.5 | μM | 1.54 | μM | 7.19 | μM | 2.45 | μM | ND | |
| 16 | ND | | ND | | ND | | >50 | μM | ND | |
| 17 | ND | | ND | | ND | | >50 | μM | ND | |
| 18 | ND | | ND | | ND | | >50 | μM | ND | |
| 19 | 12.75 | μM | 23.27 | μM | 22.06 | μM | 14.35 | μM | 5.6 | μM |
| 20 | 11.13 | μM | 5.14 | μM | 16.66 | μM | 21.41 | μM | 27.4 | μM |
| 21 | >25 | μM | >25 | μM | ND | | >25 | μM | ND | |
| 22 | 5.31 | μM | 5.53 | μM | 9.09 | μM | 1.16 | μM | 0.79 | μM |
| 23 | >25 | μM | >25 | μM | 9.93 | μM | 26.26 | μM | 2.64 | μM |
| 24 | >50 | μM | >50 | μM | 21 | μM | 10.97 | μM | 3.43 | μM |
| 25 | 1.65 | μM | 3.39 | μM | 19.04 | μM | 1.4 | μM | 5.25 | μM |
| 26 | 0.82 | μM | 12.66 | μM | ND | | 9.94 | μM | 33.66 | μM |
| 27 | 1.5 | μM | 3.9 | μM | 9.93 | μM | 2.46 | μM | ND | |
| 28 | 14.83 | μM | 23.87 | μM | 17.3 | μM | 31.34 | μM | 15.80 | μM |

| | | | | | | | | | |
|---|---|---|---|---|---|---|---|---|---|
| 29 | 5.5 | μM | 8.3 | μM | 3.96 | μM | ND | 1.46 | μM |

| 30 | ND | | 7.5 | μM | 7.5 | μM | ND | ND |
|---|---|---|---|---|---|---|---|---|
| 31 | ND | | >25 | μM | >15 | μM | ND | ND |
| 32 | ND | | 2 | μM | 1 | μM | ND | ND |
| 33 | 16 | μM | 32 | μM | 10.78 | μM | ND | ND |

| 34 | ND | >50 | μM | 16.76 | μM | ND | | 6.62 | μM |
|---|---|---|---|---|---|---|---|---|---|
| 35 | ND | 17.02 | μM | 5.4 | μM | 5.95 | μM | 1.03 | μM |
| 36 | ND | 0.32 | μM | 0.27 | μM | 0.23 | μM | 0.07 | μM |
| 37 | ND | 11.84 | μM | 9.11 | μM | 7.13 | μM | 1.22 | μM |
| 38 | ND | 6.78 | μM | 2.56 | μM | 1.61 | μM | 0.2 | μM |
| 39 | ND | 16.12 | μM | 16.1 | μM | 5.83 | μM | 1.47 | μM |
| 40 | ND | 13.27 | μM | 4.98 | μM | 5.06 | μM | 0.95 | μM |
| 41 | ND | ND | | 6.5 | μM | 3.68 | μM | 2.21 | μM |
| 42 | ND | 8.04 | μM | 7.06 | μM | 3.62 | μM | 1.84 | μM |
| 43 | ND | 0.53 | μM | 2.35 | μM | 1.64 | μM | 1.35 | μM |
| 44 | ND | 0.43 | μM | 7.12 | μM | 4.43 | μM | 2.66 | μM |

II.2. Cold Hypoxia/Reoxygenation Assays In Vitro

Model

Renal tubular necrosis that occurs during ischemia-reperfusion is a cell death by ferroptosis [Linkermann et al., PNAS, 2014, 11(47) 16836-16841]. In order to mimic the ischemia-reperfusion that occurs in an organ during transplantation, we treated kidney cells with increasing concentrations of the compounds of interest diluted in University of Wisconsin (UW) solution before their incubation in 95% N₂/5% CO₂ atmosphere at 4° C. for 24 h. Note here that UW is the most commonly used solution in hospitals for the preservation of organs before transplantation. After 24 h, the culture medium was replaced by a PBS buffer solution or the culture medium recommended for these kidney cells. The cells were then incubated for 6 h in the incubator at 37° C., in the presence of oxygen and 5% CO₂. The remaining cell viability was quantified using MTS reduction assay. Hypoxia-induced cell mortality was then estimated by quantifying the decrease in cell viability. The molecules were considered to be effective if they maintain a high and significant level of cell viability following hypoxic shock.

Material and Methods

Human renal glomerular endothelial cells (hRGEC) were grown to 80% confluence, and then synchronized using FBS (fetal bovine serum) depleted media for 16 h (M200 medium+2% serum). For hypothermia/hypoxia, cells were washed twice with PBS then incubated in UW solution in 95% N₂/5% CO₂ (Bactal 2 gaz, Air Liquide France) atmosphere at 4° C. for 24 h. Cells were treated with increasing concentrations of inhibitors at the hypoxia step. For the reoxygenation step, cells were washed and incubated in PBS+2% serum or in M200 medium+4% serum in 5% CO₂ and 21% 02 atmosphere at 37° C. for 6 h. Controls were cells not subjected to this protocol and were continuously oxygenated, incubated in PBS supplemented with 2% of FBS. Cell viability was evaluated by XTT test (Sigma-Aldrich, St Quentin-Fallavier, France) after 6 h of reoxygenation.

Results

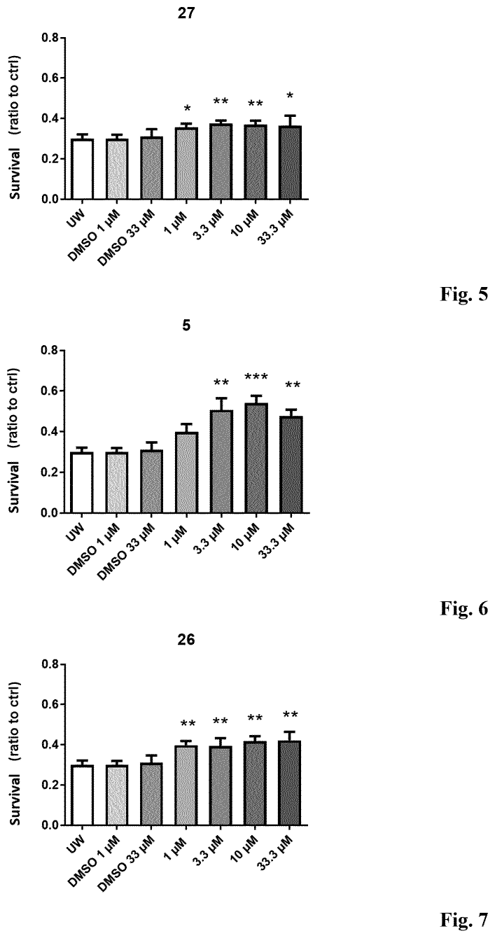

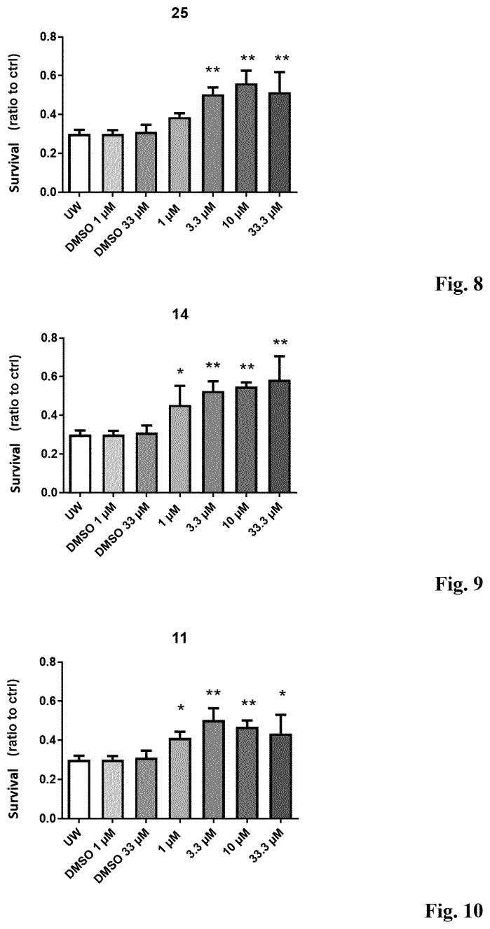

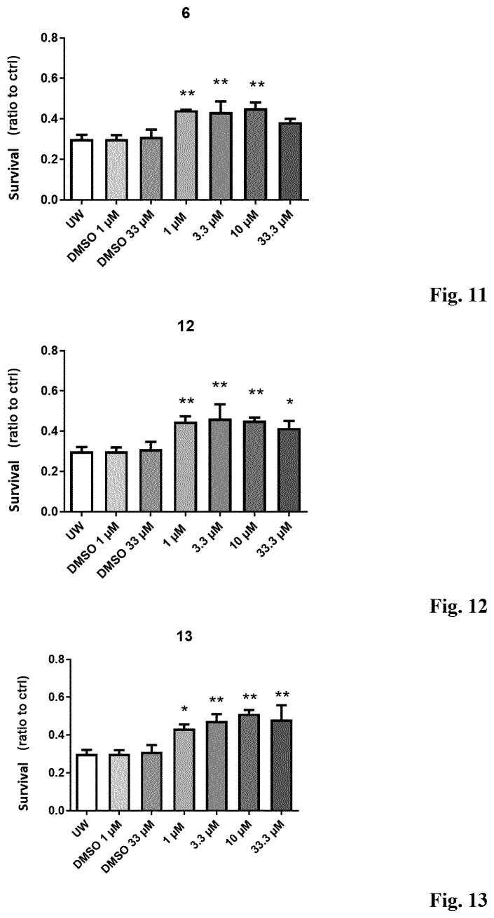

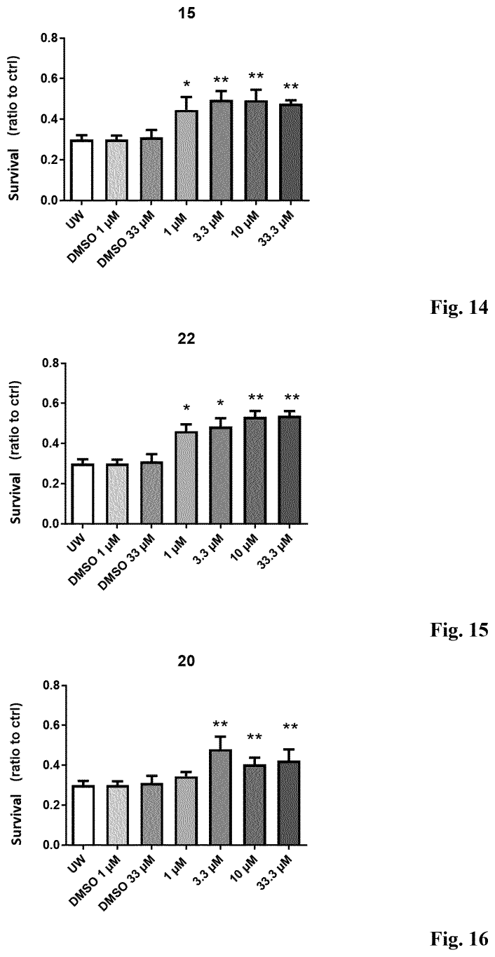

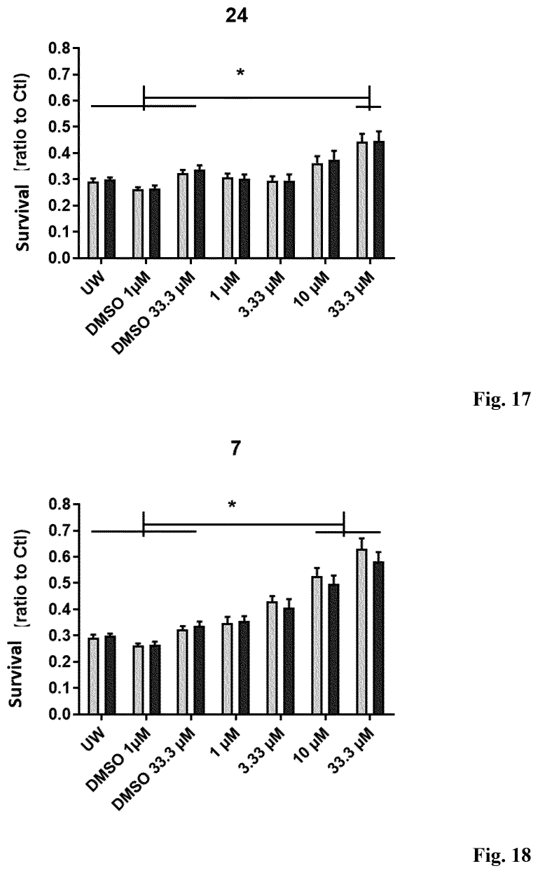

Each of the 14 compounds tested on this model had a dose-dependent cytoprotective effect, as shown in FIGS. 4-18.

II.3. Cisplatin-Induced Nephrotoxicity

Model

Nephrotoxicity associated with the use of cisplatin in the treatments of various cancers (in particular ENT and lung cancers) is due to the accumulation of cisplatin in the kidneys before its elimination. High cisplatin concentration in the kidney is known to trigger cell death by ferroptosis, causing acute tubular necrosis leading to acute kidney injury [Hu et al., Cell Death Dis., 2020, 11:73].

In order to mimic this pathology, we used non-cancerous rat proximal tubular kidney cells (NRK-52E) to which we added a high dose of cisplatin (200 μM), in the presence of increasing doses of inhibitors. We used ferrostatin-1 (Fer-1) as a positive control for ferroptosis inhibition. After 24 hours of treatment, a cell viability assay (MTS) was performed.

Material and Methods

Rat renal proximal tubular epithelial cell line (NRK-52E) were grown in high glucose Dulbecco's Modified Eagle Medium (DMEM, ATCC) supplemented with 5% FBS. Cells were maintained at 37° C. under 5% CO₂ atmosphere. NRK-52E cells were seeded in a 96-well plate at 10,000 cells/well. Cells were treated with 200 μM cisplatin (Sigma) for 24 h in presence of increasing doses of inhibitors or DMSO for control cells. Cell viability was assessed by MTS assay (CellTiter 96 AQueous One Solution Cell Proliferation assay, Promega).

Results

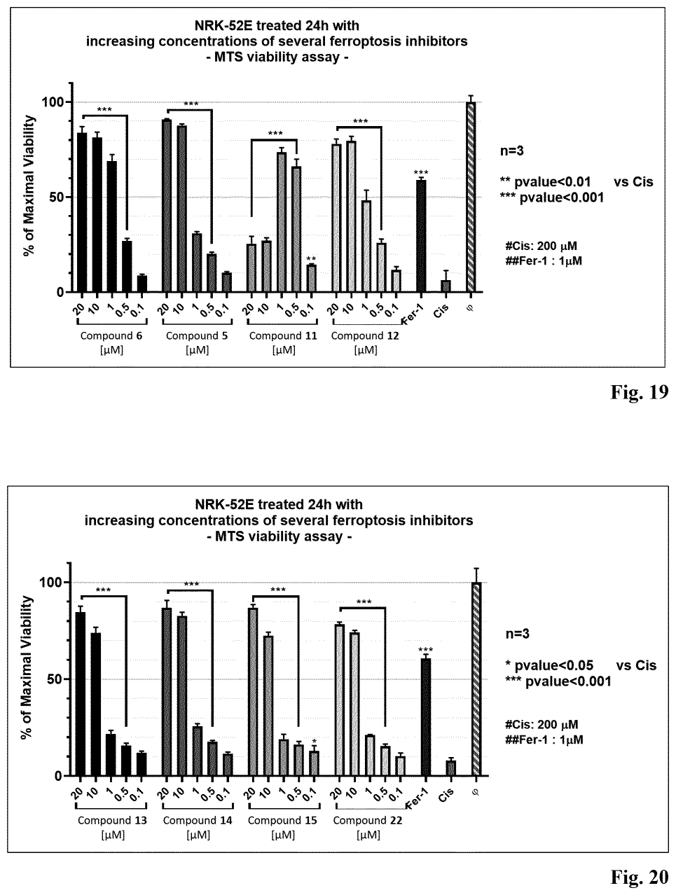

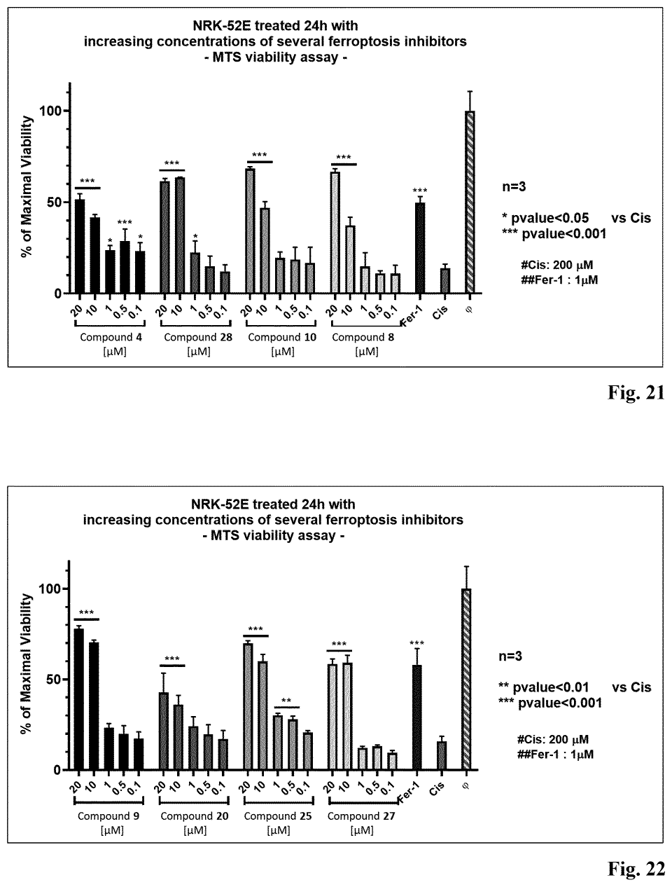

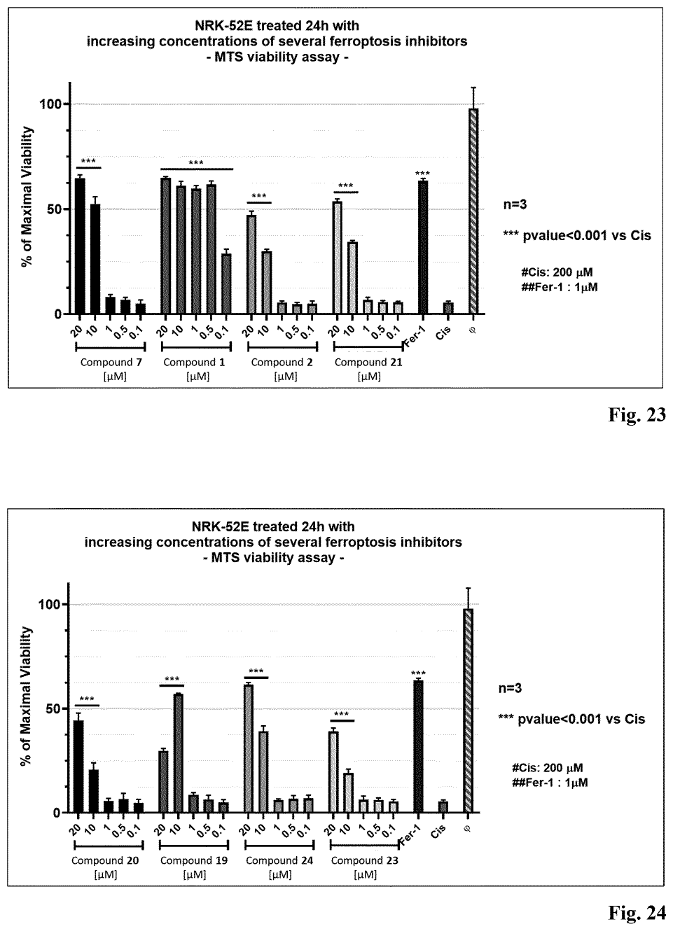

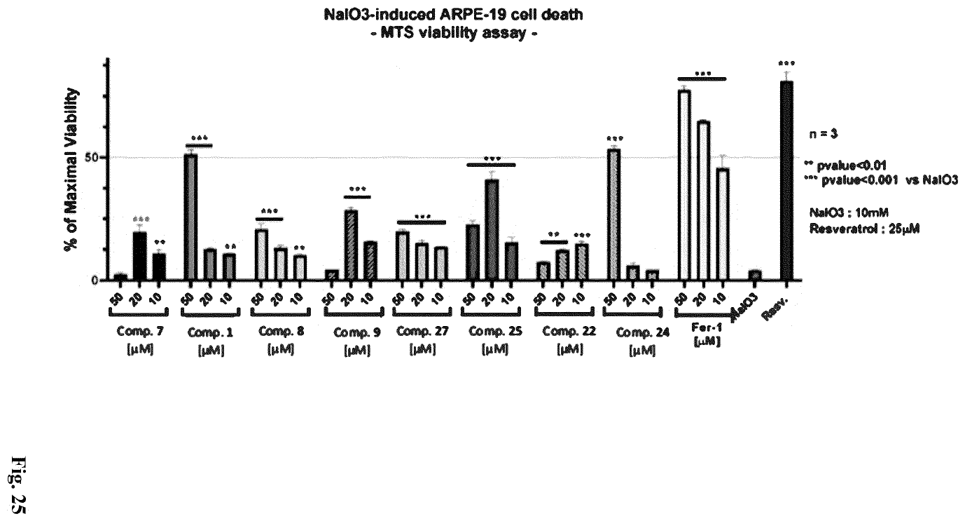

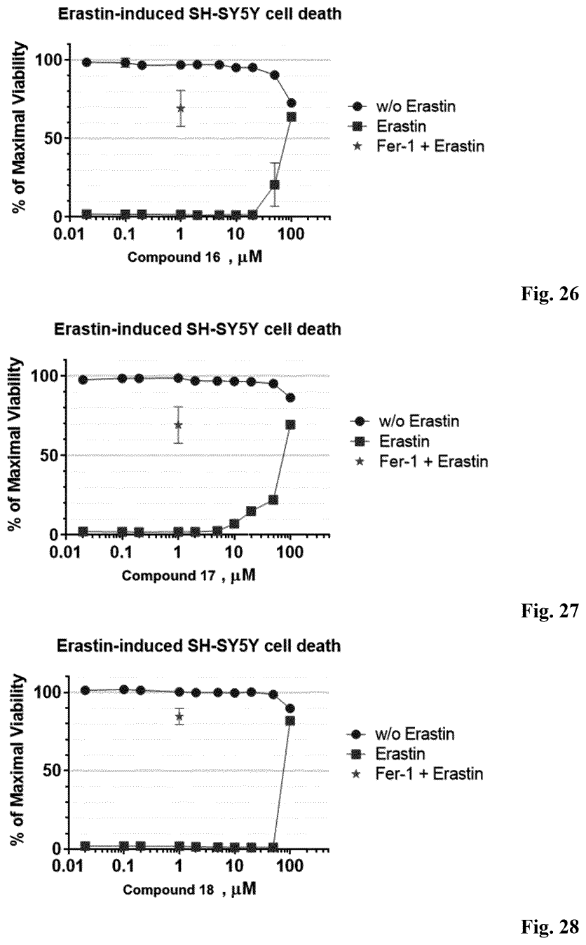

Each of the 24 compounds tested on this model had a dose-dependent cytoprotective effect against cisplastin-induced cell death, as shown in FIGS. 19-24.

II.4. Cellular Model of Age-Related Macular Degeneration (AMD)

Model

Age-related macular degeneration, or AMD, is characterized by a loss of vision caused by a degeneration of the central cells of the retina, called the macula. Oxidative stress has been shown to play an important role in the loss of retinal cells and notably by triggering ferroptosis [Totsuka et al., Exp. Eye Res., 2019, 181-316-324]. One model used to study retinal cell death is the human ARPE-19 cell line, a pigmentary retinal epithelial cell line, in the presence of sodium iodate (NaIO₃, a potent oxidizing agent) [Hanus et al. Cell Death Discov. 2016, 2, 16054] [Chan et al., J Biomed. Sci., 2019, 26:40]. Resveratrol was used as a positive control, insofar it inhibits ferroptosis by playing its well-documented role as an antioxidant agent.

Material and Methods

ARPE-19 cells were seeded in a 96-well plate at 10,000 cells/well. Cells were treated with 10 mM of NaIO₃ in presence of increasing doses of inhibitors, or resveratrol or ferrostatin-1 for control cells. Cell viability was assessed by MTS assay (CellTiter 96 AQueous One Solution Cell Proliferation assay, Promega).

Results

As it appeared from FIG. 25, the tested compounds showed a significant protective effect on ARPE-19 cells against death induced by NaIO₃ (n=3, mean±SD, *** P<0.001).

II.5. Cellular Model of Retinal Pigment Epithelium (RPE) Phototoxicity

Material and Methods

Primary cultures of porcine RPE are prepared weekly and run on 96-well culture plates after one week in vitro. The cells are incubated with the molecule 10 minutes before induction of phototoxicity. A2E (30 μM) is added to the culture medium 19 hours before cell illumination by a device equipped with blue light emitting leds (λ=430 nM). Cell survival and death are detected 24 hours after induction of phototoxicity by staining cells with Hoechst (a nuclear marker) and ethidium (a marker for dead cell nuclei). Images of each well are acquired on a fluorescence microscope equipped with a motorised stage driven by Metamorph software, and cell survival is quantified by inhouse program. The molecules are tested at 10 and 30 μM unless toxicity at this concentration. The negative control corresponds to cells treated with A2E and vehicle. The positive control corresponds to cells treated with A2E and crocetin (100 μM) 48 h before illumination. The treatments are done in quadruplicate and the experiments are performed 4 times.

Results

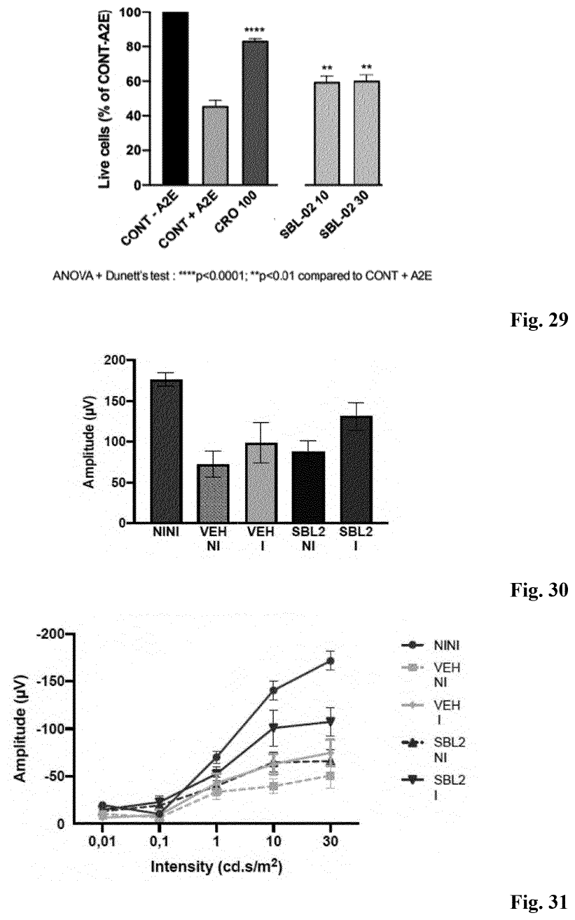

The test results depicted in FIG. 29 show that compound 1 (named SBL-02 in FIG. 29) is able to protect RPE cells significantly against phototoxicity at 10 and 30 μM.

II.6. In Vivo Model of Stargardt's Disease and Age-Related Macular Degeneration (AMD)

Model

As described in Parmar et al., Invest Ophthalmol Vis Sci., 2016, 57(7), mice lacking ATP-binding cassette transporter 4 (ABCA4) and retinol dehydrogenase 8 (RDH8) mimic features of human Stargardt's disease and age-related macular degeneration. Intense light exposure can accelerate retinal degeneration in Abca4^{-/-}Rdh8^{-/-} mice.

Experimental Protocol

Blue light damage (BLD) experiments are performed on 7-week-old Abca4−/−Rdh8−/− mice. The animals were adapted to low light levels (4000 lux, 30 minutes). Six days after BLD, an optical coherence tomography (OCT) examination of each eye is performed, which provides a “snapshot” of the state of the retinal cell layers in living mice. OCT is based on the use of a low coherence interferometer. This imaging technique allows in vivo cross-sectional images of tissues to be taken with a resolution of a few microns. It is performed under light gas anaesthesia (5% isoflurane at induction then 2% during maintenance) and is non-invasive. The pupils of the mice are dilated with 0.5% Midriaticum. A representative image of each eye was extracted and included in separate report documents (4 OCT attachments). The following day, measurements of the electrical activity of the retina were made by measuring scotopic and photopic electroretinograms (ERG). The scotopic ERG is performed after a night in the dark in order to measure the electrical response of the rods which represent 97% of the photoreceptors in the mouse. At higher intensities, the cones also respond. This is followed by a photopic ERG which records the response of the cones only. Three measurements corresponding to the activity of 1) rod and cone photoreceptors, 2) inner retinal bipolar cells and 3) cones only, are obtained. The eyes are then harvested and prepared for subsequent histological analysis. In the IP experiments, one eye is dissected and the retina and RPE are frozen separately. These tissues can be used for further measurements.

Intravitreal Treatment with Compound 1

Compound 1 in powder form is taken up in DMSO in order to obtain a solution at 20 mM. These solutions are stored at −20° C. and are thawed extemporaneously to prepare a solution diluted 89 times in PBS. These solutions give a final concentration in the eye of around 50 μM for compound 1, and 0.25% DMSO. A vehicle is prepared from pure DMSO to achieve a final 0.25% in the vitreous. Mice are lightly anaesthetised with isoflurane during the IVT procedure. A volume of 1.5 μL is injected into the vitreous of the left eye.

Results for ERG

Effect of intravitreal administration of compound 1 (named SBL2 in FIG. 30-32) on induced retinal phototoxicity in Abca4−/−Rdh8−/− mice Scotopic and photopic ERG measurements show that intravitreal injection of compound 1 at 50 μM in Abca4−/−Rdh8−/− mice appears to preserve the electrical response of retinal cells. FIG. 30 shows the results expressed as histograms by intensity for the scotopic and photopic ERGs. When the scotopic A wave of the SBL2-injected eye (SBL2 I) is compared to that of the uninjected eye (SBL2 NI), there is a noticeable difference at 10 and 30 cd·s/m2 (FIG. 31). This is also observed, when comparing the vehicle-injected eye (VEH I) to the uninjected eye (VEH NI). When the response of the SBL2-injected eye is compared to that of the vehicle-injected eye, a clear difference is observed and becomes significant (P=0.0206) when SBL2 I is compared to VEH NI at 10 cd·s/m2. The scotopic B wave appears preserved in injected eyes compared to uninjected eyes in SBL2-treated mice (FIG. 32). At 30 cd·s/m2, there was a significant difference in SBL2 I (p=0.048) compared to VEH NI. At 30 cd·s/m2, the B-wave amplitude of SBL2 I is very close to that of the NINI (untreated and non-injected) mice. The photopic ERG of the injected eyes is partially preserved compared to that of the non-injected eyes (FIG. 30). The same was true for SBL2 I compared to VEH I.

Quantification of Whole Retina Thickness from OCT

In order to measure the thickness of the retina and the photoreceptor layer, image processing was performed using Image J software. The thickness of the whole retina is measured between the outer segment layer and the ganglion cell layer. The thickness of the photoreceptor layer is measured between the outer plexiform layer and the outer segment layer. On average 6 measurements are made along the length of an OCT image representing an upper or lower half-retina. In order to prepare plates of sections of the eyes from the SBL2 IVT experiment, one eye corresponding to each condition was cut on the cryostat, i.e. one NINI eye and both eyes of an SBL2-injected mouse (I and NI). Quantification of the thickness of the retinas of SBL2 IVT-treated mice shows a significant difference between the thickness of SBL2-injected versus non-injected eyes of the same mice. The obtained results are shown in FIG. 33.

Intraperitoneal Treatment with Compound 7

The solutions are prepared extemporaneously from water-soluble lyophilisates of compound 7 (SBL1)-cyclodextrin (final CD concentration, 15%) and the corresponding placebos. The solutions are prepared by injecting a predefined volume of 0.9% NaCl into the vial. The vial is then vortexed and the solution is filtered into a new sterile tube. Two groups of 3 mice are used for these tests. The group received either SBL1 (20 mg/kg) (n=3) or placebo-SBL1 (n=3). The volume to be injected is calculated according to the weight of the mice and corresponds to 10 ml/kg. Mice were injected 3 and 1 h before and 1 and 3 h after the start of the BLD.

Results for ERG

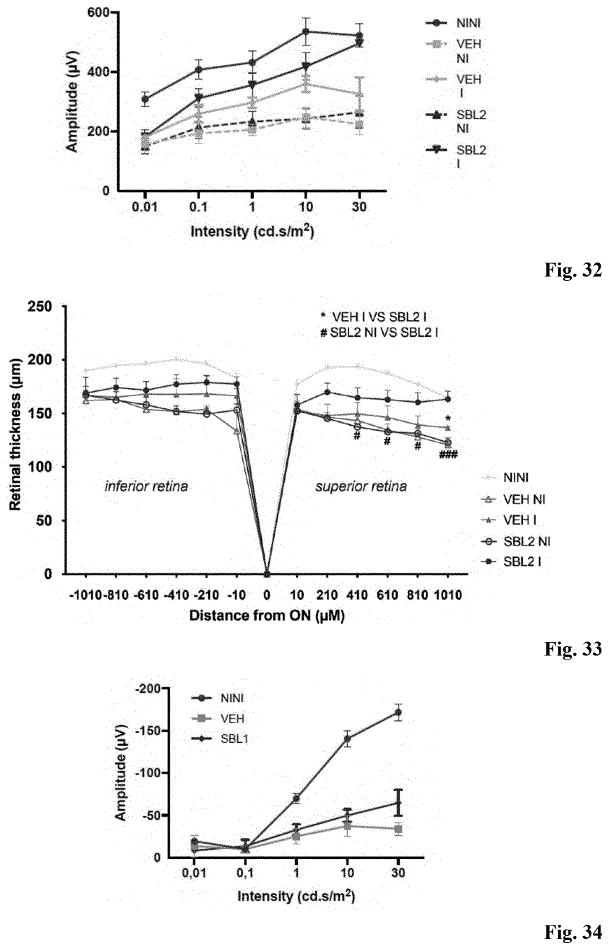

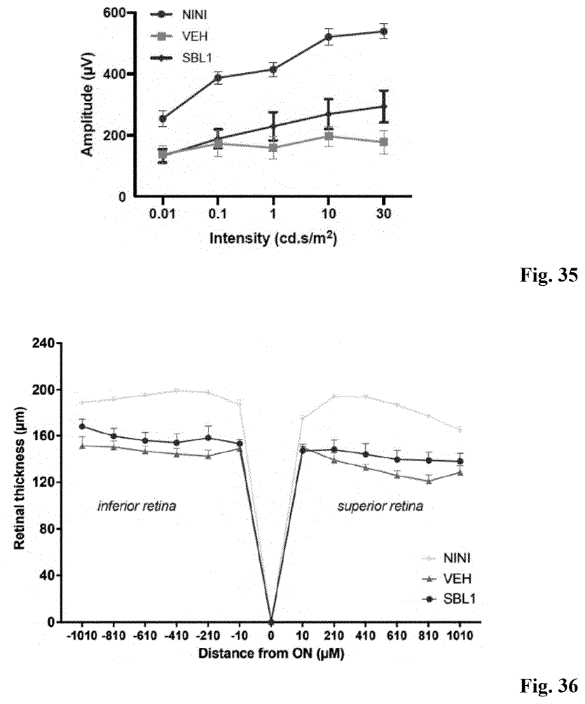

Measurements of scotopic and photopic ERGs show that multiple injections of SBL1 at 20 mg/kg before and after BLD induction in Abca4−/−Rdh8−/− mice slightly protect the electrical response of retinal cells compared to that of mice given placebo. A protective trend was observed in response to the 30 cd·s/m² flash intensity, which corresponds to a mixed “rod-cone” response with a high proportion of cones (FIGS. 34 & 35). At higher intensities of the scotopic B wave, corresponding to the response of the inner retina and in particular that of the bipolar cells, a protective trend was observed. This would correspond to the response of the inner retina stimulated by that of the mixed photoreceptor response (FIG. 35).

Quantification of Whole Retinal Thickness from OCT

Quantification of retinal thickness in SBL1 IP-treated mice shows a difference between the whole retinas of injected and non-injected mice (FIG. 36).

II.7. Protective Effect of the Compounds after 6-OHDA Injury on Rat Primary Dopaminergic Neurons Survival

Model

As explained in Ding et al., J Neurochem., 2004, (89), administration of 6-Hydroxydopamine (6-OHDA), a neurotoxin, has been widely used to cause the selective degeneration of dopamine (DA) neurons in in vitro and in vivo models. These models are used as Parkinson's disease models, which is characterized by the progressive loss of DA neurons of the substantia nigra.

Material and Methods

Rat dopaminergic neurons were cultured as described by Schinelli et al., J Neurochem., 1988, 50(6). The cells were seeded at a density of 40 000 cells/well in 96 well-plates (precoated with poly-D-lysine; Greiner, Ref: 655940, Batch: E20093UL) and were cultured at 37° C. in a humidified air (95%)/CO₂ (5%) atmosphere. Half of the medium was changed every 2 days with fresh medium. In these conditions, after 5 days of culture, astrocytes are present in the culture and release growth factor allowing neurons differentiation. In this condition, 2 to 5% of neurons are dopaminergic neurons.

6-OHDA (Sigma, Ref: 162957, Batch MKCK5243) was reconstituted in define culture medium at 40 μM (stock solution). The control medium was prepared in the same conditions. After 7 days of culture, primary mesencephalic neurons were pre-treated for 1 hour with test compounds or reference compound (BDNF, 50 ng/mL) and then intoxicated with 6-OHDA at a final concentration of 20 μM for 2 days incubation in order to induce a neuronal cell death of about 50%+/−5%.

After 2 days of intoxication in presence or absence of test compounds, cells were fixed by a solution of 4% paraformaldehyde (Sigma, ref 6148, Batch: SZBE2390V) for 20 min at room temperature, the control conditions were fixed as well following the same procedure. The cells were then permeabilized and nonspecific sites were blocked with a solution of phosphate buffered saline (PBS; PanBiotech; ref: P04-36500, Batch: 7701020) containing 0.1% of saponin (Sigma; ref: S7900, Batch: BCBL8667V) and 1% FCS for 15 min at room temperature. Cells were incubated with a mouse monoclonal anti-Tyrosine Hydroxylase (TH, 1/10 000, Sigma; ref: T1299, Batch: 014M4835) antibody in a solution of PBS overnight at 4° C. Antibody against TH stained dopaminergic neurons. Staining was revealed with the addition of an Alexa Fluor 488 goat anti-mouse IgG (1/400, Molecular probe, ref: A11001, Batch: 2247988) in PBS with 1% FCS and 0.1% saponin for 1 hour at room temperature. Nuclei of cells were labelled by a fluorescent marker (Hoechst, Sigma; ref: B1155, Batch: 046M4048V) in the same solution. For each condition, 20 pictures per well were taken using InCell Analyzer™ 2200 (GE Healthcare) with 20× magnification. Images of each culture well were taken in same condition. Analysis of cell bodies of TH positive neurons was performed using Developer software (GE healthcare). A total of 6 data per experimental condition were provided.

Results

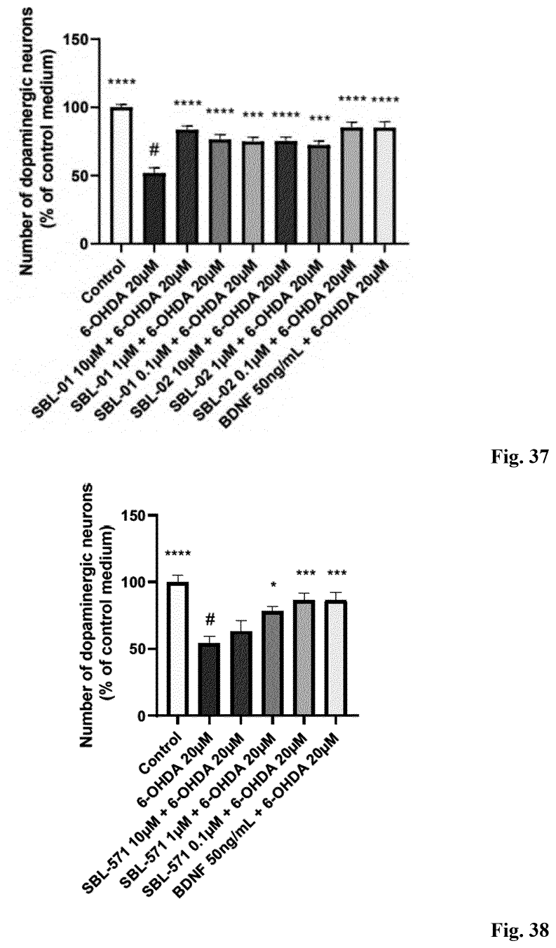

As observed on FIG. 37, 6-OHDA at 20 μM induces a significant decrease of dopaminergic neurons survival (48% of cellular death, p<0.0001). As expected, the reference molecule, BDNF at 50 ng/mL, is able to partially rescue neurons from cell death (15% of cellular death, p<0.0001). These results allow validating the culture conditions. A treatment with compound 7 (SBL-01), one hour before and during the 2 days of 6-OHDA exposure, at every concentration tested of 10 μM, 1 μM and 0.1 μM is able to partially and significantly rescue. dopaminergic neurons from cell death (respectively 16% with p<0.001, 24% with p<0.001). Similarly, a treatment with compound 1 (SBL-02), one hour before and during the 2 days of 6-OHDA injury, at every concentration tested of 10 μM, 1 μM and 0.1 μM is able to partially and significantly rescue dopaminergic neurons from cell death (respectively 25% with p<0.001).

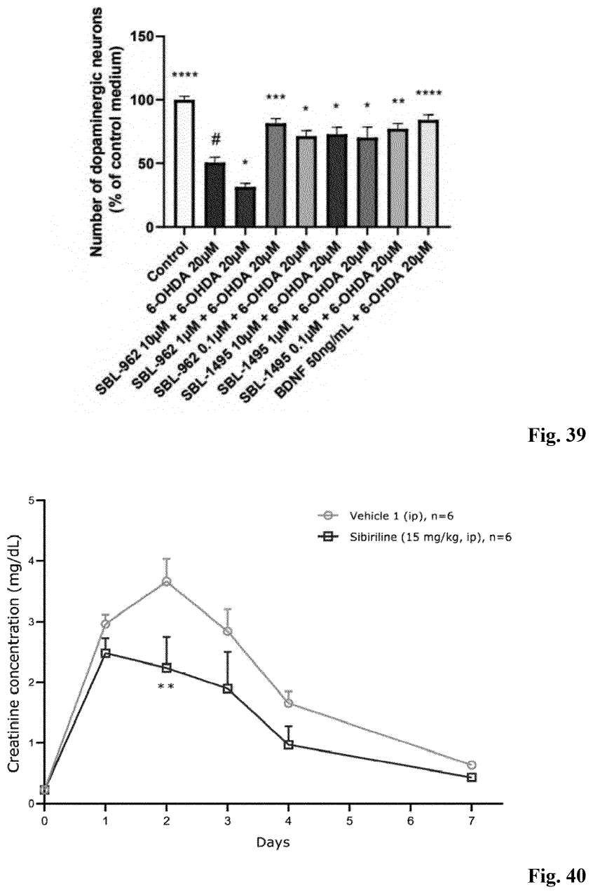

Results obtained with compounds 14 (SBL-571), 22 (SBL-962) and 3 (SBL-1495) are shown in FIGS. 38 and 39 (mean±s.e.m; *p<0.05, **p<0.01, ***p<0.001, ****p<0.0001 stats vs 6-OHDA; one way Anova followed by Dunnett's test. #represents the condition of intoxication).

II.8. In Vivo Model of Ischaemic Acute Renal Failure Induced by Transient Bilateral Renal Artery Occlusion in the Rat

Material and Methods

The experiment was carried out using male Wistar rats (Janvier Labs, C.S. 4105, Saint-Berthevin F-53941, France), weighing 200-260 g on the day of surgery. Before surgery, the animals were housed in groups 2-4 in polysulfone cages (floor area=1500 cm²) under standard conditions: room temperature (22±2° C.), hygrometry (55±10%), light/dark cycle (12 h/12 h), air replacement (15-20 volumes/hour), water and food (SAFE, ref. A04) ad libitum. The animals were allowed to acclimate to environmental conditions for at least 5 days prior to experimentation. Rats were anesthetised with sodium pentobarbital (60 mg/kg i.p.), and placed on a servocontrolled heating table to maintain rectal temperature close to 37° C. After endotracheal intubation animals were mechanically ventilated. The abdominal cavity was exposed by medial laparotomy, both left and right renal arteries will be identified, carefully dissected from the renal vein and clamped for 40 min. During this clamping period the abdominal cavity was kept closed. The clamps were released, perfusion to the kidneys re-established and the abdominal cavity sutured. Animals where reperfusion were incomplete, as judged visually, were excluded. Post-surgical analgesia was ensured by subcutaneous administration of buprenorphine (10 50 μg/kg, sc) twice daily for 2 days (including the day of surgery). After surgery, the animals were individually housed. The animals were observed and weighted daily and the sutures were examined and disinfected with antiseptic solution (povidone-iodine) if needed.

A follow-up of body weight and mortality were performed for all animals, the day of surgery and during the 7 days following ischaemia. In order to determine creatinine and blood urea nitrogen (BUN) concentrations in plasma, 6 blood samples of at least 0.3 mL were taken from each animal: before surgery and then on days 1, 2, 3 (before vehicle/SBL01 administrations), 4 and 7 after surgery. The following parameters were measured: Creatinine concentration in plasma, Blood urea nitrogen concentration, and Body weight.

Administration protocol: Using a group size of n=10, the animals received the following treatments: Vehicle 1 (5% Tween 80 in Phosphate Buffer, IP)/compound 7 (15 mg/kg, IP)

All treatments were administered via the intraperitoneal route, with an administration volume of 5 mL/kg. Administrations take place: 10 min before ischemia (=T−10 min: the treatments will be directly administered in the abdominal cavity before the renal arteries are clamped), then immediately after reperfusion (=T45 min: the treatments will be directly administered in the abdominal cavity before the abdominal cavity is sutured), and then 3 h, 24 h and 48 h after the start of ischaemia. At the end of the experiments, the deeply anaesthetised animals were sacrificed by cervical dislocation. Compound 7 was prepared in 5% Tween in phosphate buffer as vehicle.

Results

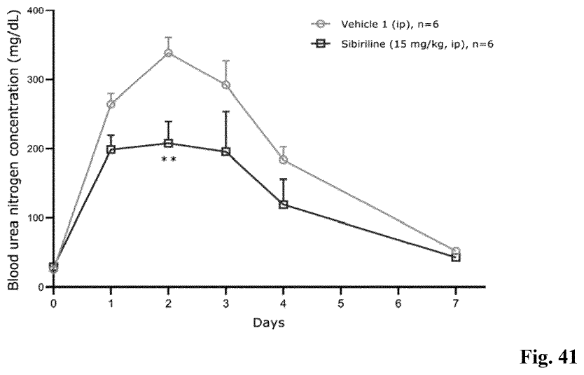

As shown in FIGS. 40 and 41, a significant decrease of the creatinine and urea plasma level at Day 2 after compound 7 (sibiriline) administration.

These results highlighted a protective effect of compound 7 on renal function during artery occlusion and thus a protection against renal ischaemic failure compared to the vehicle.

1-16. (canceled)

17. A method for inhibiting ferroptosis comprising administering to a patient in need thereof an effective amount of a compound of the following general formula (I): 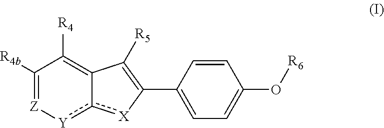
or a pharmaceutically acceptable salt and/or solvate thereof,
wherein: 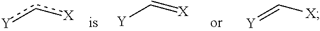
(i) when 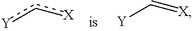
X is N, Y is N(R₂) and Z is C(H);
(ii) when is 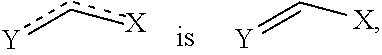
X is N(R₁), and
Y is N or N⁺(O⁻) and Z is C(R₃), or
Y is CH and Z is N, or
Y and Z are CH;
and wherein:
R₁ and R₂ represent, independently of each other, a hydrogen atom, CN, NO₂, OR₇, SR₈, NR₉R₁₀, C(O)R₁₁, CO₂R₁₂, OC(O)R₁₃, NR₁₄C(O)R₁₅, C(O)NR₁₆R₁₇, S(O)R_{S}, SO₂R_{S}′, a (C₁-C₆)alkyl, a (C₁-C₆)haloalkyl, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, CN, NO₂, OR₁₈, SR₁₉, NR₂₀R₂₁, C(O)R₂₂, CO₂R₂₃, OC(O)R₂₄, NR₂₅C(O)R₂₆, C(O)NR₂₇R₂₈, a (C₁-C₆)alkyl and a (C₁-C₆)haloalkyl group;
R₃, R₄, R_{4b} and R₅ represent, independently of each other, a hydrogen atom, a halogen atom, CN, OR₂₉, SR₃₀, NR₃₁R₃₂, C(O)R₃₃, CO₂R₃₄, OC(O)R₃₅, NR₃₆C(O)R₃₇, C(O)NR₃₈R₃₉, a (C₁-C₆)alkyl, a (C₁-C₆)haloalkyl group, said alkyl or haloalkyl group being optionally substituted by one or more substituents selected from the group consisting of OR₄₀, SR₄₁ and NR₄₂R₄₃, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, CN, NO₂, OR₄₄, SR₄₅, NR₄₆R₄₇, C(O)R₄₈, CO₂R₄₉, OC(O)R₅₀, NR₅₁C(O)R₅₂, C(O)NR₅₃R₅₄, a (C₁-C₆)alkyl and a (C₁-C₆)haloalkyl group;
R₆ represents a hydrogen atom, a (C₁-C₆)alkyl or an aryl-(C₁-C₆)alkyl group, or a (C₁-C₆)alkylcarbonyl group optionally substituted with one or more substituents selected from the group consisting of OH, SH, NH₂, a (C₁-C₆)alkoxy, a (C₁-C₆)thioalkoxy, a (C₁-C₆)alkylamino and a di((C₁-C₆)alkyl)amino group;
R_{S} and R_{S}′ represent, independently of each other a (C₁-C₆)alkyl, an aryl or an aryl-(C₁-C₆)alkyl group;
R₇-R₁₀, R₁₂, R₁₄ and R₁₆-R₁₇ represent, independently of each other, a hydrogen atom, a (C₁-C₆)alkyl, an aryl or an aryl-(C₁-C₆)alkyl group;
R₁₁, R₁₃ and R₁₅ represent, independently of each other, a hydrogen atom, a (C₁-C₆)alkyl, an aryl, an aryl-(C₁-C₆)alkyl, a (C₁-C₆)alkoxy, a (C₁-C₆)alkylamino or a di((C₁-C₆)alkyl)amino group;
R₁₈ to R₂₈ represent, independently of each other, a hydrogen atom, a (C₁-C₆)alkyl or an aryl group;
R₂₉ to R₃₉ represent, independently of each other, a hydrogen atom, a (C₁-C₆)alkyl, an aryl or an aryl-(C₁-C₆)alkyl group, said aryl group being optionally substituted by one or more substituents selected from the group consisting of a halogen atom, CN, NO₂, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group;
R₄₀ to R₄₃ represent, independently of each other, a hydrogen atom or a (C₁-C₆)alkyl group;
R₄₄ to R₅₄ represent, independently of each other, a hydrogen atom, a (C₁-C₆)alkyl or an aryl group; and
R₅₅ to R₆₅ represent, independently of each other, a hydrogen atom, a (C₁-C₆)alkyl or an aryl group.

18. The method of claim 17, for preventing and/or treating a disorder associated with ferroptosis.

19. The method of claim 17, wherein:
R₁ represents a hydrogen atom, CN, NO₂, OR₇, SR₈, NR₉R₁₀, C(O)R₁₁, CO₂R₁₂, OC(O)R₁₃, NR₁₄C(O)R₁₅, C(O)NR₁₆R₁₇, S(O)R_{S}, SO₂R_{S}′, a (C₁-C₆)alkyl, a (C₁-C₆)haloalkyl, an aryl, a heterocyclyl, an aryl-(C₁-C₆)alkyl or a heterocyclyl-(C₁-C₆)alkyl group, wherein said aryl or heterocyclyl group is optionally substituted by one substituent selected from the group consisting of a halogen atom, CN, NO₂, OR₁₈, SR₁₉, NR₂₀R₂₁, a (C₁-C₆)alkyl and a (C₁-C₆)haloalkyl group;
R_{S} and R_{S}′ represent, independently of each other, a (C₁-C₆)alkyl or an aryl group;
and
R₁₈ to R₂₁ represent, independently of each other, a hydrogen atom or a (C₁-C₆)alkyl group.

20. The method of claim 17, wherein:
R₂ represents C(O)R₁₁, CO₂R₁₂, C(O)NR₁₆R₁₇, a (C₁-C₆)alkyl, a (C₁-C₆)haloalkyl, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom OR₁₈, SR₁₉, NR₂₀R₂₁, a (C₁-C₆)alkyl and a (C₁-C₆)haloalkyl group;
and
R₁₈ to R₂₁ represent, independently of each other, a hydrogen atom or a (C₁-C₆)alkyl group.

21. The method of claim 17, wherein:
R₃ represents a hydrogen atom, a halogen atom, CN, OR₂₉, SR₃₀, NR₃₁R₃₂, C(O)R₃₃, CO₂R₃₄, OC(O)R₃₅, NR₃₆C(O)R₃₇, C(O)NR₃₈R₃₉, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, OR₄₄, SR₄₅, NR₄₆R₄₇, a (C₁-C₆)alkyl and a (C₁-C₆)haloalkyl group;
R₂₉ to R₃₇ represent, independently of each other, a hydrogen atom or a (C₁-C₆)alkyl group;
and
R₄₄ to R₄₇ represent, independently of each other, a hydrogen atom or a (C₁-C₆)alkyl group.

22. The method of claim 17, wherein:
R₄ represents a hydrogen atom, a halogen atom, CN, OR₂₉, SR₃₀, NR₃₁R₃₂, a (C₁-C₆)alkyl, a (C₁-C₆)haloalkyl group, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, CN, NO₂, OR₄₄, SR₄₅, NR₄₆R₄₇, C(O)R₄₈, CO₂R₄₉, OC(O)R₅₀, NR₅₁C(O)R₅₂, C(O)NR₅₃R₅₄, a (C₁-C₆)alkyl and a (C₁-C₆)haloalkyl group;
R_{4b} represents a hydrogen atom, a halogen atom, OR₂₉ or NR₃₁R₃₂;
R₂₉ to R₃₂ represent, independently of each other, a hydrogen atom, a (C₁-C₆)alkyl, an aryl or an aryl-(C₁-C₆)alkyl group, said aryl group being optionally substituted by one or more substituents selected from the group consisting of a halogen atom, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group, notably C(O)R₅₉, CO₂R₆₀, C(O)NR₆₄R₆₅, a (C₁-C₆)alkyl and a (C₁-C₆)haloalkyl group, in particular C(O)R₅₉;
R₄₈ represents a hydrogen atom, a (C₁-C₆)alkyl or an aryl group, notably an aryl group;
and
R₅₅ to R₆₅ represent, independently of each other, a hydrogen atom, a (C₁-C₆)alkyl or an aryl group, notably an aryl group.

23. The method of claim 17, wherein:
R₅ represents a hydrogen atom, a halogen atom, CN, OR₂₉, SR₃₀, NR₃₁R₃₂, a (C₁-C₆)alkyl, a (C₁-C₆)haloalkyl group, said alkyl or haloalkyl group being optionally substituted by one or more substituents selected from the group consisting of OR₄₀, SR₄₁ and NR₄₂R₄₃, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, OR₄₄, SR₄₅, NR₄₆R₄₇, a (C₁-C₆)alkyl and a (C₁-C₆)haloalkyl group;
R₂₉ to R₃₂ represent, independently of each other, a hydrogen atom or a (C₁-C₆)alkyl group;
and
R₄₀ to R₄₃ represent, independently of each other, a hydrogen atom or a (C₁-C₆)alkyl group, notably a hydrogen atom.

24. The method of claim 17, wherein R₆ represents a hydrogen atom, a (C₁-C₃)alkyl or an aryl-(C₁-C₃)alkyl group, or a (C₁-C₆)alkylcarbonyl group optionally substituted with one or more substituents selected from the group consisting of OH, SH, NH₂, a (C₁-C₃)alkoxy, a (C₁-C₃)thioalkoxy and a (C₁-C₃)alkylamino group.

25. The method of claim 17, wherein the compound is selected from the 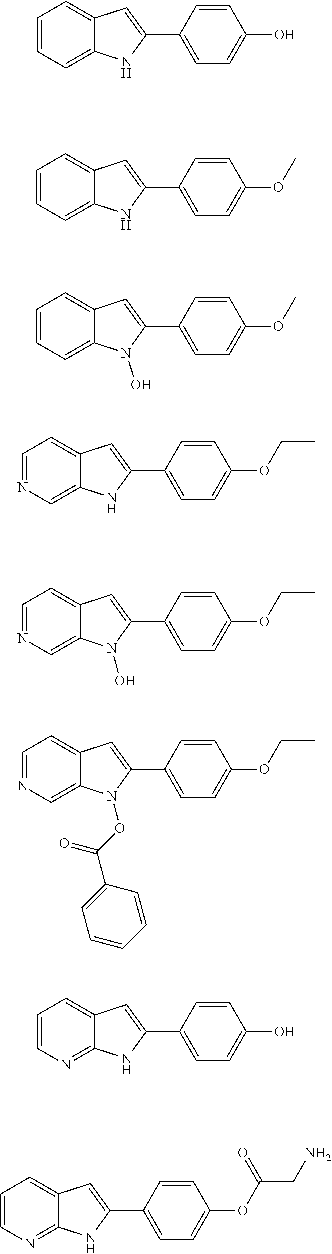 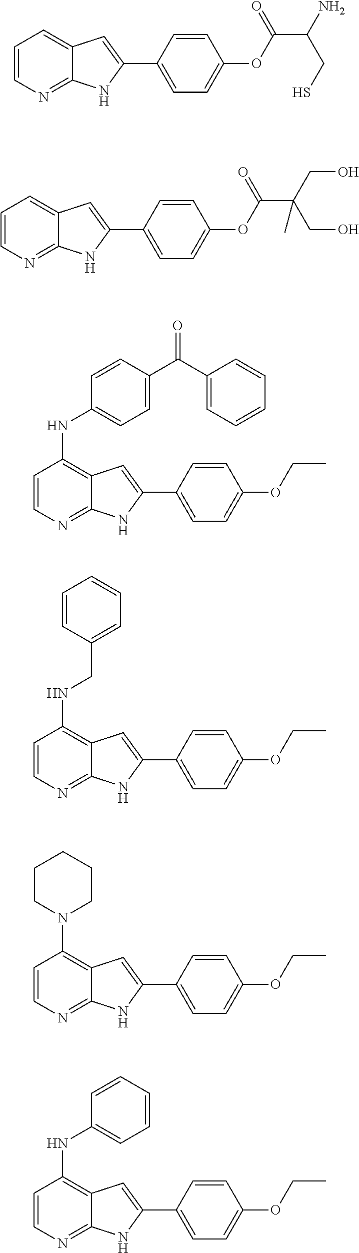 [SMILES: NC(CS)C(=O)Oc1ccc(-c2cc3cccnc3[nH]2)cc1 ; CC(CO)(CO)C(=O)Oc1ccc(-c2cc3cccnc3[nH]2)cc1 ; CCOc1ccc(-c2cc3c(Nc4ccc(C(=O)c5ccccc5)cc4)ccnc3[nH]2)cc1 ; CCOc1ccc(-c2cc3c(NCc4ccccc4)ccnc3[nH]2)cc1 ; CCOc1ccc(-c2cc3c(N4CCCCC4)ccnc3[nH]2)cc1 ; CCOc1ccc(-c2cc3c(Nc4ccccc4)ccnc3[nH]2)cc1] 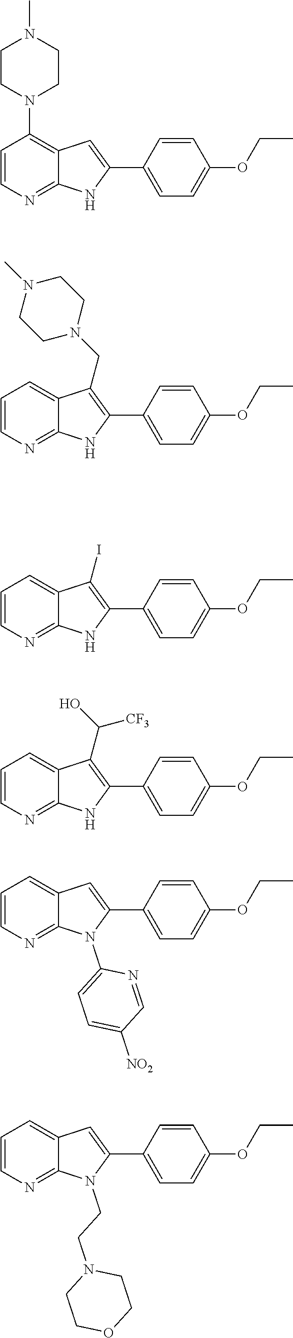 [SMILES: CCOc1ccc(-c2cc3c(N4CCN(C)CC4)ccnc3[nH]2)cc1 ; CCOc1ccc(-c2[nH]c3ncccc3c2CN2CCN(C)CC2)cc1 ; CCOc1ccc(-c2[nH]c3ncccc3c2I)cc1 ; CCOc1ccc(-c2[nH]c3ncccc3c2C(O)C(F)(F)F)cc1 ; CCOc1ccc(-c2cc3cccnc3n2-c2ccc([N+](=O)[O-])cn2)cc1 ; CCOc1ccc(-c2cc3cccnc3n2CCN2CCOCC2)cc1] 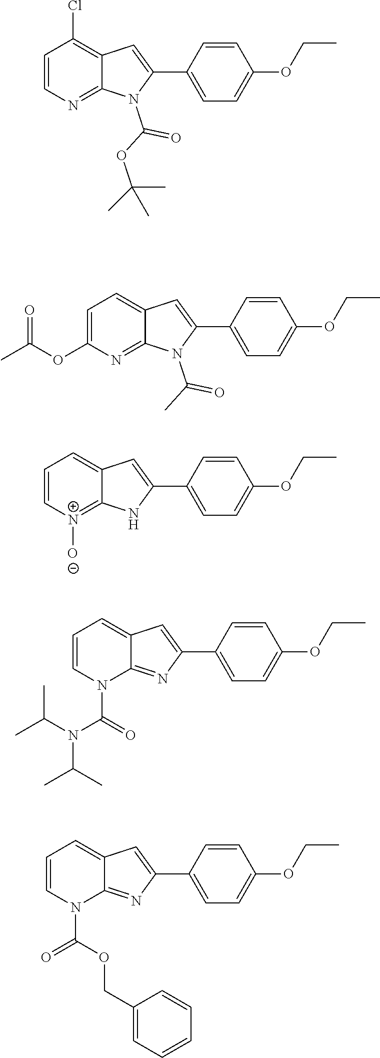 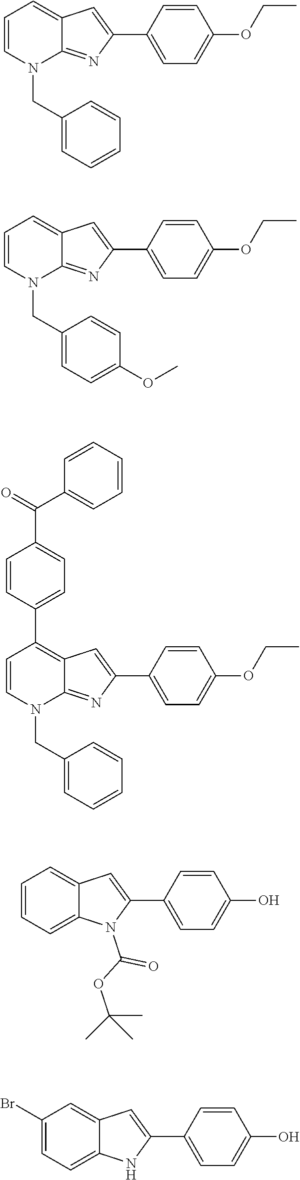 [SMILES: CCOc1ccc(-c2cc3cccn(Cc4ccccc4)c-3n2)cc1 ; CCOc1ccc(-c2cc3cccn(Cc4ccc(OC)cc4)c-3n2)cc1 ; CCOc1ccc(-c2cc3c(-c4ccc(C(=O)c5ccccc5)cc4)ccn(Cc4ccccc4)c-3n2)cc1 ; CC(C)(C)OC(=O)n1c(-c2ccc(O)cc2)cc2ccccc21 ; Oc1ccc(-c2cc3cc(Br)ccc3[nH]2)cc1] 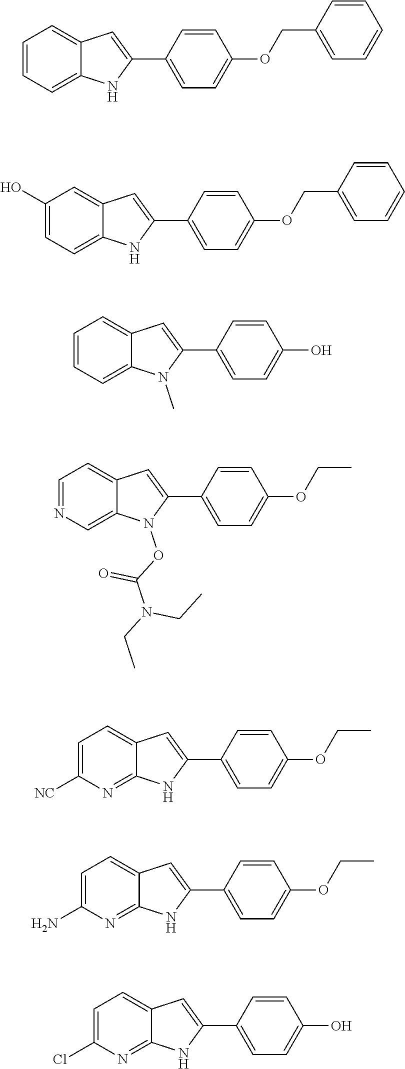 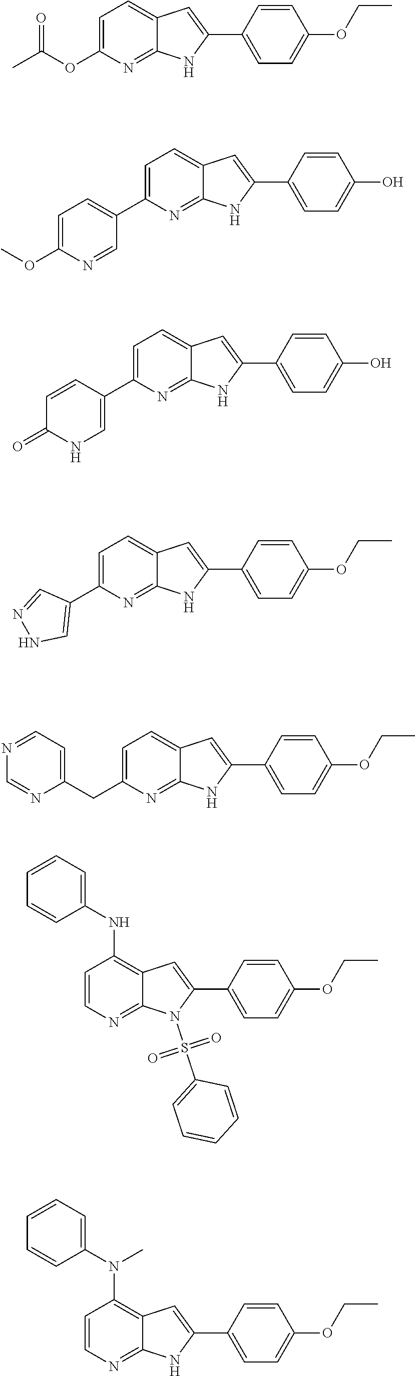
and the pharmaceutically acceptable salts and/or solvates thereof.

26. The method of claim 17, wherein the compound is selected from the 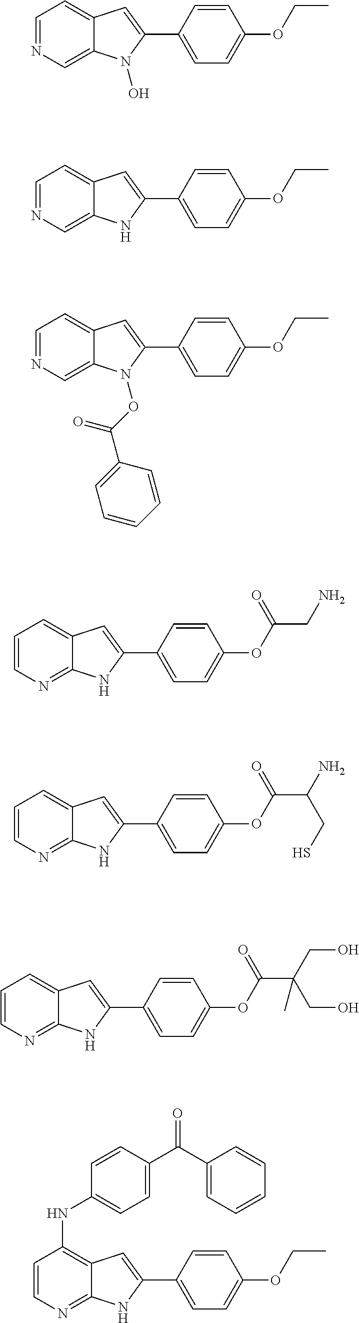 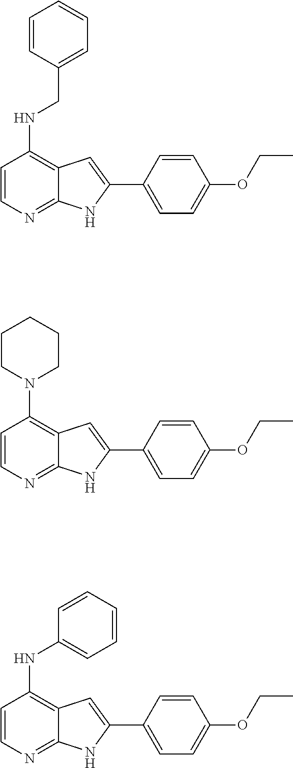 [SMILES: CCOc1ccc(-c2cc3c(NCc4ccccc4)ccnc3[nH]2)cc1 ; CCOc1ccc(-c2cc3c(N4CCCCC4)ccnc3[nH]2)cc1 ; CCOc1ccc(-c2cc3c(Nc4ccccc4)ccnc3[nH]2)cc1] 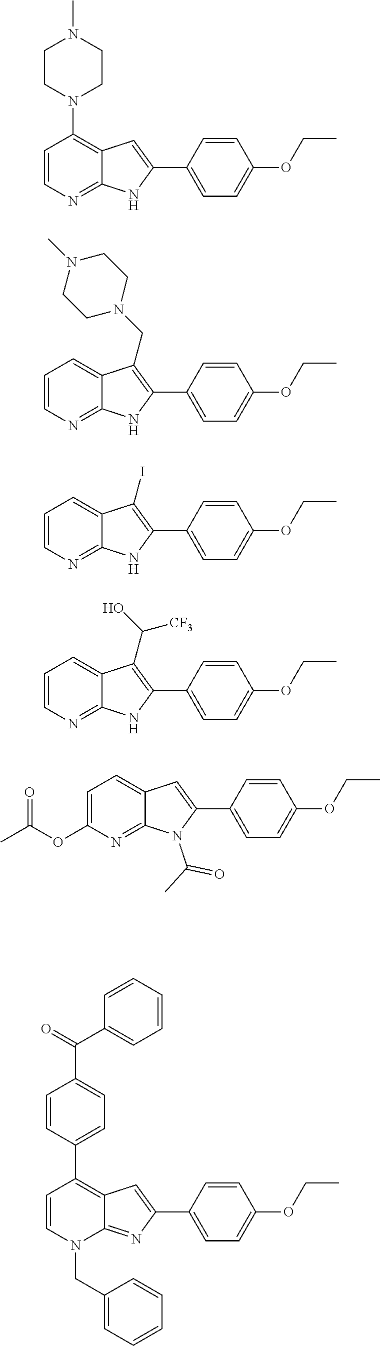 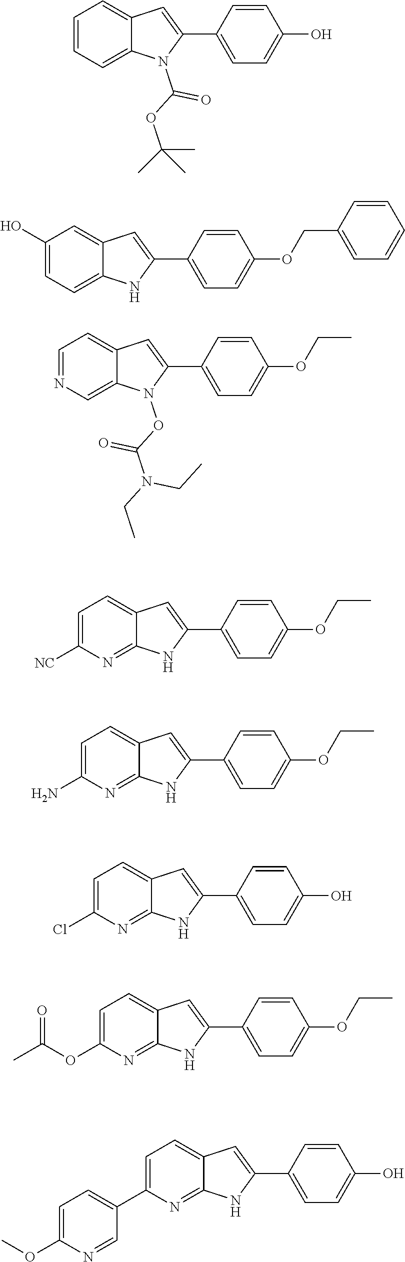 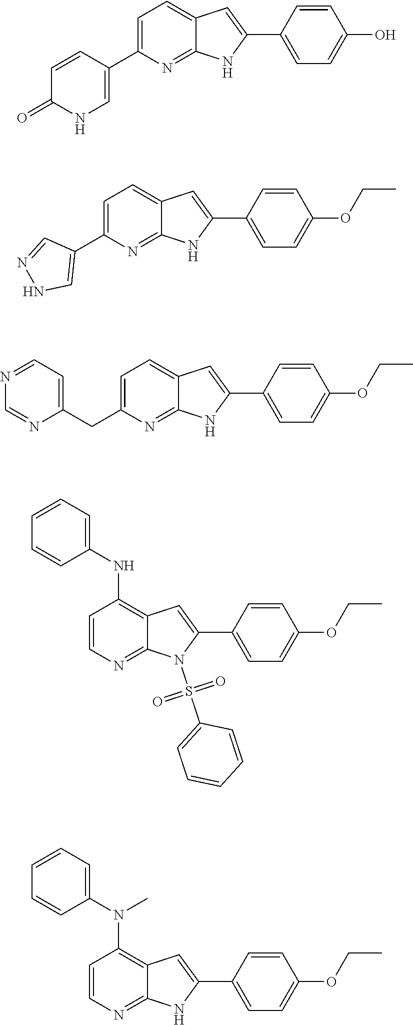 [SMILES: O=c1ccc(-c2ccc3cc(-c4ccc(O)cc4)[nH]c3n2)c[nH]1 ; CCOc1ccc(-c2cc3ccc(-c4cn[nH]c4)nc3[nH]2)cc1 ; CCOc1ccc(-c2cc3ccc(Cc4ccncn4)nc3[nH]2)cc1 ; CCOc1ccc(-c2cc3c(Nc4ccccc4)ccnc3n2S(=O)(=O)c2ccccc2)cc1 ; CCOc1ccc(-c2cc3c(N(C)c4ccccc4)ccnc3[nH]2)cc1]
and the pharmaceutically acceptable salts and/or solvates thereof.

27. The method of claim 18, wherein said disorder associated with ferroptosis is selected from the group consisting of myocardial ischemia-reperfusion injury, notably occurring after artery ligation; cardiomyopathy, notably doxorubicin-induced cardiomyopathy; strokes, notably ischemic stroke or hemorrhagic stroke; traumatic brain injury; contusion spinal cord injury; neurodegenerative disorders, in particular chronic neurodegenerative disorders, more particularly Alzheimer's disease, Huntington's disease, Parkinson's disease, amyotrophic lateral sclerosis (Charcot's disease), Friedreich's ataxia and dementia; retinal disorders, notably Stargardt's disease or age-related macular degeneration (AMD), in particular dry AMD; chronic liver diseases, notably non-alcoholic steatohepatitis (NASH), chronic infections, acute liver failure, notably resulting from a drug-induced liver injury (DILI), skin inflammatory diseases, acute kidney injury (AKI), chronic obstructive pulmonary disease (COPD); bronchial asthma; lung injury caused by a bacterial infection, notably by Pseudomonas aeruginosa or Mycobacterium tuberculosis; pulmonary fibrosis, necrotizing enterocolitis; inflammatory bowel diseases, hemolytic disorders; cytokinic storm during a viral infection; radiation-induced necrosis; rheumatoid arthritis; type I diabetes; insulin resistance related to obesity; epilepsy, including mitochondrial disease-related epilepsy and intractable epilepsy; and pathologies related to stress-induced premature tissue senescence.

28. The method of claim 18, wherein said disorder associated with ferroptosis is selected from the group consisting of cardiomyopathy, notably doxorubicin-induced cardiomyopathy; contusion spinal cord injury; neurodegenerative disorders, in particular chronic neurodegenerative disorders, more particularly Alzheimer's disease, Huntington's disease, Parkinson's disease, amyotrophic lateral sclerosis (Charcot's disease), Friedreich's ataxia and dementia; retinal disorders, notably Stargardt's disease or age-related macular degeneration (AMD), in particular dry AMD; acute liver failure, notably resulting from a drug-induced liver injury (DILI), skin inflammatory diseases, acute kidney injury (AKI), such as oxalate-, folic acid (FA)- and cisplatin-induced AKI; bronchial asthma; lung injury caused by a bacterial infection, notably by Pseudomonas aeruginosa or Mycobacterium tuberculosis; pulmonary fibrosis, necrotizing enterocolitis; haemochromatosis; hemolytic disorders; cytokinic storm during a viral infection; radiation-induced necrosis; rheumatoid arthritis; type I diabetes; insulin resistance related to obesity; epilepsy, including mitochondrial disease-related epilepsy and intractable epilepsy; and pathologies related to stress-induced premature tissue senescence.

29. The method of claim 18, wherein said disorder associated with ferroptosis is selected from the group consisting of myocardial ischemia-reperfusion injury, notably occurring after artery ligation; strokes, notably ischemic stroke or hemorrhagic stroke; traumatic brain injury; neurodegenerative disorders, in particular chronic neurodegenerative disorders, more particularly Alzheimer's disease, Huntington's disease, Parkinson's disease and amyotrophic lateral sclerosis (Charcot's disease); retinal disorders, notably Stargardt's disease or age-related macular degeneration (AMD), in particular dry AMD; chronic liver diseases, notably non-alcoholic steatohepatitis (NASH); acute liver failure, notably resulting from a drug-induced liver injury (DILI), and acute kidney injury (AKI).

30. An in vitro method for inhibiting ferroptosis comprising adding a compound as defined in claim 17 to a biological material, thereby inhibiting ferroptosis-induced cell death in the biological material.

31. A compound selected from the group consisting of: 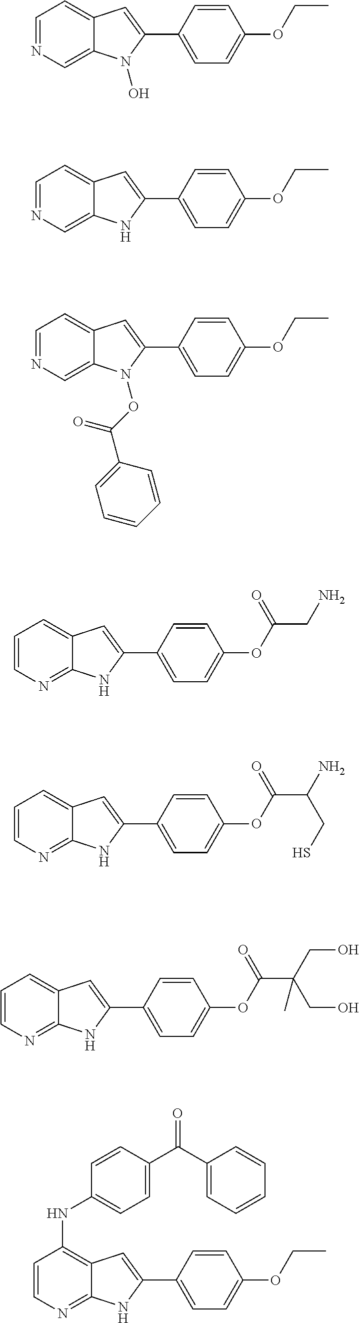 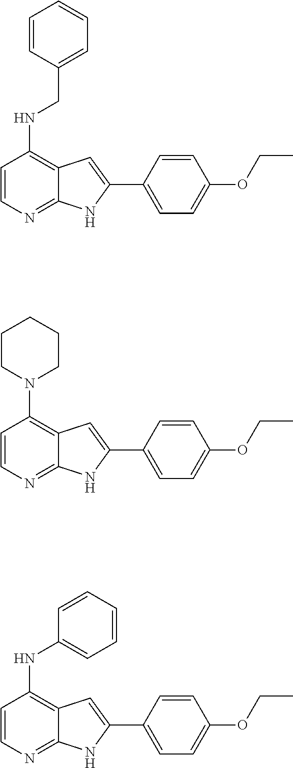 [SMILES: CCOc1ccc(-c2cc3c(NCc4ccccc4)ccnc3[nH]2)cc1 ; CCOc1ccc(-c2cc3c(N4CCCCC4)ccnc3[nH]2)cc1 ; CCOc1ccc(-c2cc3c(Nc4ccccc4)ccnc3[nH]2)cc1] 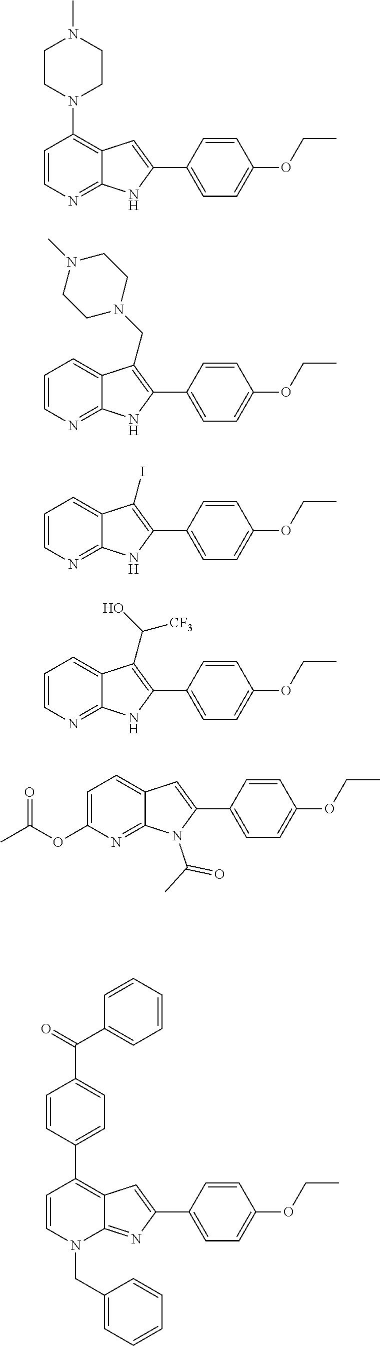 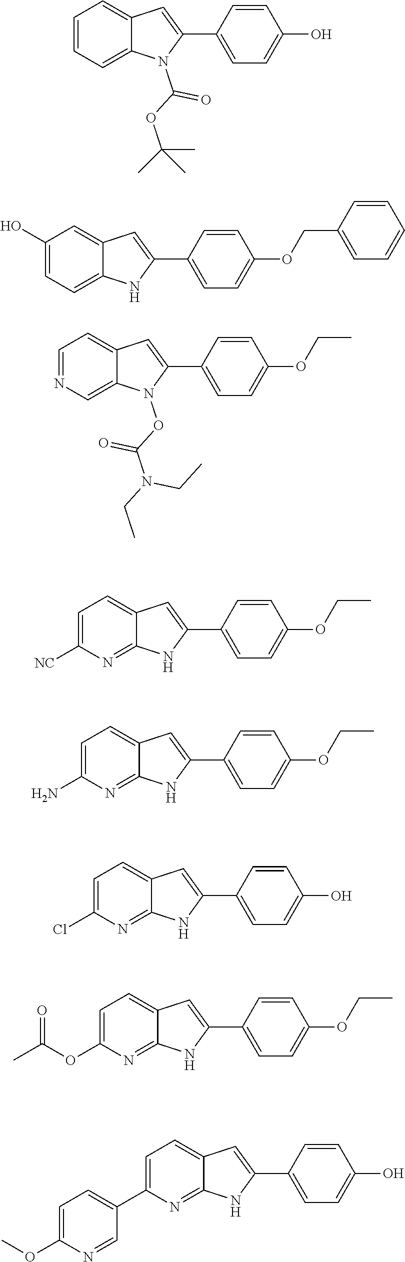 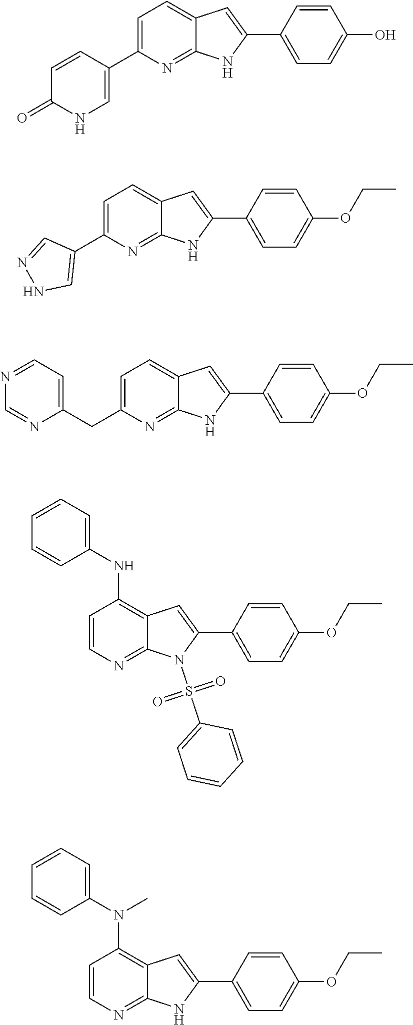 [SMILES: O=c1ccc(-c2ccc3cc(-c4ccc(O)cc4)[nH]c3n2)c[nH]1 ; CCOc1ccc(-c2cc3ccc(-c4cn[nH]c4)nc3[nH]2)cc1 ; CCOc1ccc(-c2cc3ccc(Cc4ccncn4)nc3[nH]2)cc1 ; CCOc1ccc(-c2cc3c(Nc4ccccc4)ccnc3n2S(=O)(=O)c2ccccc2)cc1 ; CCOc1ccc(-c2cc3c(N(C)c4ccccc4)ccnc3[nH]2)cc1]
and the pharmaceutically acceptable salts and/or solvates thereof.

32. A method for inhibiting ferroptosis comprising administering to a patient in need thereof an effective amount of a compound according to claim 31.

33. The method of claim 19, wherein
R₁ represents a hydrogen atom, CN, OR₇, C(O)R₁₁, CO₂R₁₂, OC(O)R₁₃, SO₂R_{S}′, a (C₁-C₆)alkyl, a heterocyclyl or a heterocyclyl-(C₁-C₆)alkyl group, wherein said heterocyclyl group is optionally substituted by NO₂;
R_{S} and R_{S}′ represent an aryl group.

34. The method of claim 20, wherein R₂ represents CO₂R₁₂, C(O)NR₁₆R₁₇ or an aryl-(C₁-C₆)alkyl group, wherein said aryl group is optionally substituted by one or more substituents selected from the group consisting of a halogen atom, OR₁₈, SR₁₉ and NR₂₀R₂₁.

35. The method of claim 21, wherein R₃ represents a hydrogen atom, a halogen atom, CN, OR₂₉, SR₃₀, NR₃₁R₃₂, OC(O)R₃₅, NR₃₆C(O)R₃₇, a heterocyclyl or a heterocyclyl-(C₁-C₆)alkyl group, wherein said heterocyclyl group is optionally substituted by one or more substituents selected from the group consisting of a halogen atom, OR₄₄, SR₄₅, NR₄₆R₄₇ and a (C₁-C₆)alkyl a group.

36. The method of claim 22, wherein
R₄ represents a hydrogen atom, a halogen atom, OR₂₉, SR₃₀, NR₃₁R₃₂, an aryl or a heterocyclyl group, wherein said aryl or heterocyclyl group is optionally substituted by one or more substituents selected from the group consisting of a halogen atom, OR₄₄, SR₄₅, NR₄₆R₄₇, C(O)R₄₈, CO₂R₄₉, C(O)NR₅₃R₅₄, a (C₁-C₆)alkyl and a (C₁-C₆)haloalkyl group;
R_{4b} represents a hydrogen atom.
